(19) United States
(12) Patent Application Publication
(10) Pub. No.: US 2008/0026068 A1
(43) Pub. Date: Jan. 31, 2008

(54) PULMONARY DELIVERY OF SPHERICAL INSULIN MICROPARTICLES

(75) Inventors: Larry R. Brown, Newton, MA (US); John K. McGeehan, Woodbury, NJ (US); Qin Yuanxi, Lexington, MA (US); Julia Rashba-Step, Newton, MA (US); Terrence L. Scott, Winchester, MA (US)

Correspondence Address: BAXTER HEALTHCARE CORPORATION, ONE BAXTER PARKWAY, DF2-2E, DEERFIELD, IL, 60015, US

(73) Assignees: BAXTER HEALTHCARE S.A., Wallisellen (CH); BAXTER INTERNATIONAL, Deerfield, IL (US)

(21) Appl. No.: 11/736,372

(22) Filed: Apr. 17, 2007

Related U.S. Application Data
(63) Continuation-in-part of application No. 10/222,200, filed on Aug. 16, 2002.
(63) Continuation-in-part of application No. 10/894,432, filed on Jul. 19, 2004.
(60) Provisional application No. 60/312,894, filed on Aug. 16, 2001.
(60) Provisional application No. 60/488,712, filed on Jul. 18, 2003.

(51) Int. Cl. A61K 9/14; A61K 38/28; A61P 11/00

(52) U.S. Cl. 424/489; 514/3

(57) ABSTRACT

Compositions of spherical insulin particles having improved pulmonary application potentials and methods of forming and using these compositions are disclosed in the present application. In one clinical trial with 30 healthy male human subjects, no coughing was observed upon a single pulmonary administration of the spherical insulin particles at an insulin dose of 6.5 mg, nor during the 10-hour post dosing period.

The present application is a continuation-in-part application of 10/222,200 which was filed Aug. 16, 2002, claiming the benefit of priority under 35 U.S.C. § 119(e) of U.S. provisional application 60/312,894, which was filed Aug. 16, 2001. The present application also is a continuation-in-part application of 10/894,432, Jul. 19, 2004 claiming the benefit of priority under 35 U.S.C. §119(e) of U.S. Provisional Application Ser. No. 60/488,712 which was filed Jul. 18, 2003. The entire text of each of the aforementioned applications is incorporated by reference.

### FIELD OF THE APPLICATION

The present application relates to pulmonary delivery of insulin through the use of small spherical particles of insulin.

### BACKGROUND OF THE ART

Several techniques have been used in the past for the manufacture of biopolymer nano- and microparticles. Conventional techniques include spray drying and milling for particle formation and can be used to produce particles of 5 μm or less in size.

Microparticles produced by standard production methods frequently have a wide particle size distribution, lack uniformity, fail to provide adequate release kinetics, and are difficult and expensive to produce. Frequently, the polymers used to prepare these microspheres are primarily soluble in organic solvents, requiring the use of special facilities designed to handle organic solvents. The organic solvents can denature proteins or peptides contained in the microspheres, and may also be toxic to the environment, present an inflammatory hazard, as well as being potentially toxic when administered to humans or animals. In addition, the microparticles may be large and tend to form aggregates, requiring a size selection process to remove particles considered to be too large for administration to patients by injection or inhalation. This requires sieving and resulting product loss.

U.S. Pat. No. 5,981,719, U.S. Pat. No. 5,849,884 and U.S. Pat. No. 6,090,925, the disclosures of which are incorporated by reference herein in their entirety, describe microspheres formed by combining a macromolecule, such as a protein or peptide, and a polymer in an aqueous solution at a pH at or near the isoelectric point of the protein. The solution is heated to prepare microspheres having a protein content of greater than 40%. The microspheres thus formed comprise a matrix of substantially homogeneous proteins and varying amounts of polymers, which permit the aqueous medium to enter and solubilize the components of the microspheres. The microspheres can be designed to exhibit short-term or long-term release kinetics, providing either rapid or sustained release characteristics.

U.S. Pat. No. 6,051,256 relates to processes for preparing powders of biological proteins by atomizing liquid solutions of the proteins, drying the droplets, and collecting the resulting particles. Biological proteins which reportedly can be used in this process include insulin and calcitonin.

Microparticles, microspheres, and microcapsules are solid or semi-solid particles having a diameter of less than one millimeter, more preferably less than 100 microns and most preferably less than 10 microns, which can be formed of a variety of materials, including proteins, synthetic polymers, polysaccharides and combinations thereof. Microspheres have been used in many different applications, primarily separations, diagnostics, and drug delivery.

The most well known examples of microspheres used in separations techniques are those which are formed of polymers of either synthetic or natural origin, such as polyacrylamide, hydroxyapatite or agarose. In the controlled drug delivery area, molecules are often incorporated into or encapsulated within small spherical particles or incorporated into a monolithic matrix for subsequent release. A number of different techniques are routinely used to make these microspheres from synthetic polymers, natural polymers, proteins and polysaccharides, including phase separation, solvent evaporation, coascervation, emulsification, and spray drying. Generally the polymers form the supporting structure of these microspheres, and the drug of interest is incorporated into the polymer structure.

Particles prepared using lipids to encapsulate target drugs are currently available. Liposomes are spherical particles composed of a single or multiple phospholipid and/or cholesterol bilayers. Liposomes are 100 nanometer or greater in size and may carry a variety of water-soluble or lipid-soluble drugs. For example, lipids arranged in bilayer membranes surrounding multiple aqueous compartments to form particles may be used to encapsulate water soluble drugs for subsequent delivery as described in U.S. Pat. No. 5,422,120 to Sinil Kim.

Pulmonary delivery of insulin has been used in a number of studies. Pulmonary delivery is a superior approach for the delivery of numerous classes of medicaments. The expansiveness of the aggregate lung surfaces makes the lung tissue ideal for a rapid and effective transfer of the delivered medication to the bloodstream. However, the pulmonary delivery of medicaments is not without its drawbacks. It was recently reported pulmonary complications of diabetes. It was noted that the disease is associated with increased risk of pneumonia and aspiration, that autonomic neuropathy is associated with disordered breathing during sleep as well as with decreased perception of difficulty breathing, and that there may be structural abnormalities in the lungs of persons with diabetes due to increased or abnormal collagen and elastin, all characteristically leading to subclinical lung dysfunction. (Hsia et al., Symposium: Pulmonary delivery of insulin. Program and abstracts of the 63rd Scientific Sessions of the American Diabetes Association; Jun. 13-17, 2003; New Orleans, La.). In addition, it has been shown that in hyperglycemic subjects there is particular reduction in the membrane-diffusing capacity. In type 2 diabetes, initial lung function appears normal, but there is a decrease in diffusing capacity, which is particularly manifest with exercise. From these and other studies it was shown that inhaled insulin is antigenic, increasing antibodies, decreasing CD4 T-cell responses, and may lead to antidiabetogenic CD8 gamma-delta T cells in type 1 diabetes models. These complications with the previously suggested pulmonary insulin delivery methods lead to shortness of breath, coughing and therefore lead to poor patient compliance.

Thus there is a specific need for the development of new methods for making insulin-based microparticles, particularly those that can be adapted for use in pulmonary drug delivery systems. The most desirable insulin particles from a utility standpoint would be small spherical particles that have the following characteristics: narrow size distribution, substantially spherical, substantially free of excipients (e.g., consisting of only the active agent), retention of the biochemical integrity and of the biological activity of the insulin, and high bioavailability and biopotency. The particles should provide a suitable solid that would allow additional stabilization of the particles by coating or by microencapsulation. Further, the method of fabrication of the small spherical particles would have the following desirable characteristics: simple fabrication, an essentially aqueous process, high yield, and requiring no subsequent sieving.

### SUMMARY OF THE APPLICATION

Described herein are compositions of insulin particles having improved pulmonary application potentials and methods of forming and using such compositions. Using these compositions in healthy male human subjects, no coughing was observed upon a single pulmonary administration of the spherical insulin particles at an insulin dose of 6.5 mg either immediately on administration or during the 10-hour post dosing period.

Examples in the present application describe a composition for the pulmonary delivery of insulin through a powder dispenser which composition comprises a powder that comprises a dose of insulin, the powder consisting essentially of solid, substantially spherical insulin particles, the insulin particles comprising at least 90% by weight insulin suitable for in-vivo delivery and having a density of from about 0.50 to about 2.00 g/cm³.

In some examples, the solid, small spherical microparticles of insulin have a density of from about 0.50 to about 1.5 g/cm³.

In other examples, the solid, small spherical microparticles of insulin have a density greater than 0.75 g/cm³.

In still other examples, the solid, small spherical microparticles of insulin have a density greater than 0.85 g/cm³.

In specific embodiments, compositions are provided in which the solid, small spherical microparticles further comprise an excipient to enhance the stability of the solid, small spherical particles, to provide controlled release of the solid, small spherical particle, or to enhance permeation of the solid, small spherical particles through biological tissues, the excipients being present in the microparticles at less than 5% by weight.

In some examples, the excipient is selected from the group consisting of: carbohydrates, cations, anions, amino acids, lipids, fatty acids, surfactants, triglycerides, bile acids or their salts, fatty acid esters, and polymers.

In certain aspects, the cation is selected from group consisting of Zn²⁺, Mg²⁺, and Ca²⁺. The cation also may be another inorganic cation such as Mn²⁺, Na⁺, Ba²⁺, K⁺, Co²⁺, Cu²⁺, Fe²⁺, Fe³⁺, Al³⁺ and Li+.

In some examples, at least 90% of the small spherical microparticles have size between from about 0.01 μm to about 5 μm.

In other examples, at least 90% of the small spherical microparticles have a size between from about from about 0.1 μm to about 5 μm. In yet other examples at least 90% the small spherical microparticles have a size between from about 1 μm to about 3 μm.

In compositions described herein the narrow size distribution comprises the ratio of a volume diameter of the 90^{th} percentile of the small spherical particles to the volume diameter of the 10^{th} percentile is less than or equal to about 5.0.

In specific examples, the insulin may form from about 95% to about 100% of the weight of the microparticles. In other examples, the microparticles are microspheres that comprise greater than about 99% insulin by weight.

The small spherical particles may be semi-crystalline or non-crystalline.

Specific compositions are contemplated in which the composition does not comprise a surfactant.

In other compositions, the composition is characterized in that the composition does not comprise an excipient and contains only the insulin microspheres.

Also described are compositions for the pulmonary delivery of insulin through a powder dispenser comprising a carrying member to be used in connection with the powder dispenser, the carrying member carries a powder consisting essentially of solid, substantially spherical insulin particles, the insulin particles comprising at least 90% by weight insulin suitable for in-vivo delivery and having a density of from about 0.50 to about 2.00 g/cm³.

In such compositions, the solid, small spherical microparticles of insulin have a density of from about 0.50 to about 1.5 g/cm³.

Preferably, the solid, small spherical microparticles of insulin have a density greater than 0.75 g/cm³.

In some examples, the solid, small spherical microparticles of insulin have a density greater than 0.85 g/cm³.

It is contemplated that in some examples, the solid, small spherical microparticles further comprise an excipient to enhance the stability of the solid, small spherical particles, to provide controlled release of the solid, small spherical particle, or to enhance permeation of the solid, small spherical particles through biological tissues, the excipients being present in the microparticles at less than 5% by weight.

In another example, the composition is such that it comprises an excipient is selected from the group consisting of: carbohydrates, cations, anions, amino acids, lipids, fatty acids, surfactants, triglycerides, bile acids or their salts, fatty acid esters, and polymers.

The cation is selected from group consisting of Zn²⁺, Mg²⁺, and Ca²⁺. The cation also may be another inorganic cation such as Mn²⁺, Na⁺, Ba²⁺, K⁺, Co²⁺, Cu²⁺, Fe²⁺, Fe³⁺, Al³⁺, and Li+.

In certain examples, at least 90% of the small spherical microparticles have size between from about 0.01 μm to about 5 μm.

In other examples, at least 90% of the small spherical microparticles have a size between from about from about 0.1 μm to about 5 μm.

In still other examples, at least 90% the small spherical microparticles have a size between from about 1 μm to about 3 μm.

There are some examples in which the compositions have a narrow size distribution which comprises the ratio of a volume diameter of the 90^{th} percentile of the small spherical particles to the volume diameter of the 10^{th} percentile is less than or equal to about 5.0.

In some examples, the insulin is from about 95% to about 100% by weight of the microparticles. In some examples, the microparticles are microspheres comprising greater than about 99% insulin by weight.

It is contemplated that the small spherical particles can be semi-crystalline or non-crystalline. In specific compositions for the powder dispenser that consist essentially of solid, substantially spherical insulin particles, the composition does not comprise a surfactant. In other embodiments, the composition does not comprise an excipient and contains only the insulin microspheres.

Also contemplated is a method of administering insulin to the pulmonary system of a subject, comprising: administering to the pulmonary system an amount of the composition of claim 1 effective to produce a change in the subject's serum insulin level or the subject's serum glucose level or both, wherein the administration of the composition does not produce coughing in the subject upon inhalation.

Another example is directed to a composition for pulmonary delivery of insulin, comprising a powder that comprises a dose of insulin, the powder consisting essentially of solid, substantially spherical insulin particles, the insulin particles comprising at least 90% by weight insulin suitable for in-vivo delivery and having a density of from about 0.50 to about 2.00 g/cm³, wherein the composition do not produce coughing in healthy male subjects upon pulmonary administration at an insulin dose of 6.5 mg.

Also contemplated is a method of administering insulin to the pulmonary system of a subject, comprising: administering to the respiratory tract of a subject in need of treatment, an effective amount of the composition of claim 1, wherein the administration of the composition does not produce shortness of breath in the subject upon inhalation.

In such a method, the administration preferably produces a bioavailability of the insulin bioavailability of at least 10% of the bioavailablilty produced by a subcutaneous dose.

In other examples in such methods the administration produces a bioavailability of the insulin bioavailability of at least 10% of the bioavailablilty produced by a subcutaneous dose. In other examples, the administration produces a bioavailability of the insulin bioavailability of at least 12% of the bioavailablilty produced by a subcutaneous dose. In still other examples, in such methods, the administration produces a bioavailability of the insulin bioavailability of at least 15% of the bioavailablilty produced by a subcutaneous dose.

In some aspects the present application contemplates achieving a deep lung deposition of insulin in a subject comprising administering to the pulmonary system of the subject a composition of as described herein.

### BRIEF DESCRIPTION OF THE DRAWINGS

FIG. 1a is a scanning electron micrograph (SEM) of the starting insulin material.

FIG. 1b is an SEM of a small spherical particle of insulin (Example 4).

FIG. 2 is an HPLC analysis showing overall maintenance of chemical stability of insulin when prepared into small spherical particles.

FIGS. 3a and 3b are schematics demonstrating batch-to-batch reproducibility.

FIG. 4 is a schematic demonstrating batch-to-batch reproducibility.

FIG. 5 is a schematic diagram of the continuous flow through process for making insulin small spherical particles in Example 3.

FIG. 6 is a scanning electron micrograph (at 10 Kv and 6260× magnification) of the insulin small spherical particles produced by the continuous flow through process in Example 3.

FIG. 7 is an HPLC chromatograph of dissolved insulin small spherical particles prepared by the continuous flow through process in Example 3.

FIGS. 8a-8d demonstrate the effect of sodium chloride on insulin solubility.

FIGS. 8e-8h demonstrate the effect of different salts on insulin solubility.

FIG. 8i is a Raman spectra of raw material insulin, insulin released from small spherical particles and insulin in small spherical particles.

FIG. 9 is an Andersen Cascade Impactor results for radiolabeled insulin of Example 10.

FIG. 10 is a bar graph of P/I ratios for Example 8.

FIG. 11 is a scintigraphic image of a lung from Example 8.

FIG. 12 is a plot of TSI Corporation Aerosizer particle size data.

FIG. 13 is a chart showing insulin stability data in HFA-134a.

FIG. 14 is a chart comparing aerodynamic performance of Insulin using three inhalation devices.

FIGS. 15-20 are charts of stability data of Insulin small spherical particles compared to Insulin starting material stored at 25° C. and at 37° C.

FIG. 21 is a bar graph of insulin aerodynamic stability using a Cyclohaler DPI.

FIGS. 22A-B are schematic illustrations of the continuous emulsification reactor, where FIG. 22A is a schematic illustration of the continuous emulsification reactor when surface active compound added to the continuous phase or the dispersed phase before emulsification, and FIG. 22B is a schematic illustration of the continuous emulsification reactor when the surface active compound is added after emulsification.

FIG. 23 illustrates the effect of pH of continuous phase on IVR profile of PLGA encapsulated insulin small spherical particles (Example 14).

FIG. 24 illustrates the effect of the microencapsulation variables (pH of continuous phase and matrix material) on formation of INS dimers in encapsulated INSms (Example 15).

FIG. 25 illustrates the effect of the microencapsulation variables (pH of continuous phase and matrix material) on formation of HMW species in encapsulated INSms (Example 15).

FIG. 26 illustrates in-vivo release of recombinant human insulin from unencapsulated and encapsulated pre-fabricated insulin small spherical particles in rats (Example 16).

FIG. 27 shows particle size determined by laser light scattering Coutler LS230.95% of the Insulin microspheres are between 0.95 and 1.20 microns.

FIG. 28 depicts aerodynamic diameter determined using a TSI Aerosizer (Model 322500, St. Paul, Minn.)

FIG. 29 shows Andersen Cascade Impactor studies with 10 mg insulin delivered from Aerolizer DPI (JM032701C).

FIG. 30 shows in vitro Andersen cascade impaction studies with insulin delivered from vials containing HFA P134a and HFA P227.

FIG. 31 depicts glucose depression after SC injections of Insulin microspheres in SC rats.

FIG. 32 depicts glucose depression after intratracheal instillation of Insulin microspheres.

FIG. 33 compares suspension stability.

FIG. 34 shows TC-99m insulin lung distribution in dog lung.

FIG. 35 shows an assay for content and related substances (USP).

FIG. 36 is a comparison of MDI activity 1 week and 4 months after the fill.

FIG. 37 depicts insulin microsphere administration to dog via DPI.

FIG. 38 shows percent emitted dose of Insulin microspheres from MDI.

FIG. 39 depicts insulin stability in HFA P134a.

FIG. 40 mean smoothed glucose infusion rate profiles between inhaled and subcutaneous administration of recombinant human insulin in human subjects.

FIG. 41 shows pharmacokinetic profile of RHIIP administration as compared to Actrapid® administration and demonstrates RHIIP had an earlier onset of activity and similar duration of absorption as compared to the Actrapid® subcutaneous dose.

FIG. 42 shows pharmacodynamic profile of RHIIP administration as compared to Actrapid® administration.

FIGS. 43a-c show the Cyclohaler™ DPI device used in Example 33 with the clear capsule (Vcaps™, size 3) placed in its chamber. The spherical insulin particles loaded in the capsule (white solid) are clearly visible.

FIG. 44 shows % peak area of A-21 desamido insulin and high molecular weight product (mostly insulin dimers) peaks of PROMAXX insulin microspheres stored in sealed vessels at 25° C. at 60% relative humidity.

FIG. 45 shows average % emitted dose of PROMAXX insulin microspheres stored for 8 months at different temperatures with moisture contents maintained at different levels.

FIG. 46 shows serum insulin levels over time in Beagle dogs derived following the treatments described in Example 34.

FIG. 47 shows serum glucose levels over time in Beagle dogs resulted from the treatments described in Example 34.

### DETAILED DESCRIPTION

Inhaled insulin is usually delivered with specifically developed inhalers, which are often quite large and not always easy to handle. In the present application, it is demonstrated that humans can be treated with recombinant human insulin inhalation powder (RHIIP) disclosed herein. The present application discloses the formation of uniform insulin microspheres which can be administered with a relatively small commercially available dry powder inhaler (DPI).

The insulin inhalation powder used in the studies described herein showed that unlike the pulmonary administration studies others have performed, it produced no coughing or shortness of breath in the subjects to whom the formulations were administered upon immediate inhalation. Indeed, the inhaled insulin also did not produce either coughing or shortness of breath during 10 hours of monitoring after the initial inhalation of the RHIIP. The clinical use of other inhaled insulin powders reported in the literature has been hampered with the occurrence of coughing upon dosing. This event is undesirable because coughing (which is a sharp exhalation) during or immediately after inhalation can cause a portion of the dose to be exhaled before the powder reaches the deep lung. In the case of insulin, this results in an under-dosing of drug. The cough event is generally accepted as an undesirable side effect that is a result of the inhalation of any powder. Surprisingly, the results of the clinical study disclosed herein indicate that it is possible to produce an inhaled insulin powder product that has reduced potential to induce a cough response. It does not appear that the mass of powder alone explains the lack of coughing since the mass of the insulin powder delivered was similar to other studies. Without wishing to be limited thereto, one explanation may be that the spherical insulin particles disclosed herein do not contain bulking agents (such as mannitol) or matrices of excipients that other preparations contain and it may be the non-insulin ingredients that cause the coughing. Another explanation may be that the spherical insulin particles disclosed herein may dissolve so rapidly upon contacting the lung surface that a cough response is not induced.

Another characteristic of the spherical insulin particles disclosed herein is that administration of these particles to mammalian subjects (e.g., humans and dogs) resulted in surprisingly high bioavailability in the subjects. It is generally thought that the bioavailability of inhaled insulin does not exceed 10% of a subcutaneous dose. In the clinical trial disclosed herein, the bioavailability observed was surprisingly high for subjects that received the targeted dose from the dry powder inhaler. Bioavailabilities around 30% were observed in humans, with an average bioavailability of greater than 10%. Comparable bioavailability was observed in other mammals (e.g., dogs). In specific embodiments, the compositions disclosed herein produce 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35% or greater bioavailability of the insulin in subjects. The examples herein contemplate any ranges between these specified integers. The spherical insulin particles are free of excipient matrices and are rapidly dissolved when administered, both features favoring a more efficient transport of insulin across alveolar membranes. In addition, the aerodynamic properties of the spherical insulin particles disclosed herein also favor a high percentage of the emitted dose to reach the deep lung, the primary site where dissolved insulin is readily absorbed.

Further it is found that there was indeed a higher than expected level of deposition of the spherical insulin particles in the deep lung using the inhaled insulin compositions disclosed herein. The literature states that optimal delivery to the deep lung occurs with low density particles with a large geometric diameter but an MMAD approaching one micrometer. Denser particles with a geometric diameter approaching one are deemed to have poor delivery efficiencies due to the belief that the small dense particles stick together and require unrealistically high energy from a DPI to disperse. The bioavailability data suggests that the delivery of the spherical insulin particles of the present application to the deep lung was at least as efficient as other engineered particle preparations reported in the literature, which is surprising. Without wishing to be limited thereto, one explanation may be that the spherical insulin particles disclosed herein have only slightly adhesive surfaces and do not tend to agglomerate, and the primary spherical insulin particles of around one micron are readily dispersed by a simple low energy dry powder inhaler device. In addition to effective dispersion from a simple device, the spherical insulin particles disclosed herein may allow for more efficient aerodynamic characteristics in the environment of the human lung.

The insulin microspheres described herein also exhibit improved and unexpected storage stability at ambient temperatures. The spherical insulin particle compositions disclosed herein exhibit significantly improved characteristics compared to pulmonary protein formulations suggested in the prior art, especially those that have relied on surfactant and emulsion methods to incorporate drugs. In addition, the spherical insulin particles disclosed herein are prepared without the need for spray drying or milling processes.

In one example, a microparticle composition including: a plurality of microparticles (e.g., microspheres), said microparticles containing a protein or polypeptide (collectively referred to as “protein”); and a propellant (e.g., a hydrofluoroalkane (HFA) propellant), is provided. The composition has a fine particle fraction in the range of 25% to 100%. In particularly preferred embodiments, the microparticles are microspheres which have a protein content which is in the range of 20 to 100% of the total weight of the microsphere. In general, microparticles (e.g., microspheres) that are useful for pulmonary delivery of a therapeutic protein or peptide to the lung have a diameter in the range of about 0.1μ to about 10μ (for some applications, 0.1μ to 5μ; and for other applications, 0.1μ to 3μ); and a density in the range of about 0.6 gm/cc to about 2.5 gm/cc (more preferably, 0.6 gm/cc to 1.8 gm/cc; and most preferably, 1.2 gm/cc to 1.7 gm/cc). In some preferred embodiments, the microspheres have a protein content that is at least 40% of the microsphere weight (more preferably, at least 50%, 60%, 70%, or 80%; and most preferably, at least 90%, 95% or 100%).

As used herein, the term, “microparticles” refers to microparticles, microspheres, and microcapsules, that are solid or semi-solid particles having a geometric or aerodynamic diameter of less than 100 microns, more preferably less than 10 microns, which can be formed of a variety of materials, including synthetic polymers, proteins, and polysaccharides. A number of different techniques are routinely used to make these microparticles from synthetic polymers, natural polymers, proteins and polysaccharides, including phase separation, solvent evaporation, emulsification, and spray drying. Exemplary polymers used for the formation of microspheres include homopolymers and copolymers of lactic acid and glycolic acid (PLGA) as described in U.S. Pat. No. 5,213,812 to Ruiz, U.S. Pat. No. 5,417,986 to Reid et al., U.S. Pat. No. 4,530,840 to Tice et al., U.S. Pat. No. 4,897,268 to Tice et al., U.S. Pat. No. 5,075,109 to Tice et al., U.S. Pat. No. 5,102,872 to Singh et al., U.S. Pat. No. 5,384,133 to Boyes et al., U.S. Pat. No. 5,360,610 to Tice et al., and European Patent Application Publication Number 248,531 to Southern Research Institute; block copolymers such as tetronic 908 and poloxamer 407 as described in U.S. Pat. No. 4,904,479 to Illum; and polyphosphazenes as described in U.S. Pat. No. 5,149,543 to Cohen et al.

As used herein, the term, “microspheres”, refers to microparticles that are substantially spherical in shape and that have dimensions generally of between about 0.1 microns and 10.0 microns in diameter. The microspheres disclosed herein typically exhibit a narrow size distribution, and are formed as discrete particles. Illustrative methods for forming microspheres are described below.

As used herein, an “aqueous solution”, refers to solutions of water alone, or water mixed with one or more water-miscible solvents, such as ethanol, DMSO, acetone N-methylpyrrolidone, and 2-pyrrolidone; however, the preferred aqueous solutions do not contain detectable organic solvents.

In specific embodiments, methods of production and methods of use compositions of small spherical insulin particles are disclosed herein. In accordance with the methods of production, the raw material (e.g., Zn-insulin crystals) is dissolved in a solvent containing a dissolved phase-separation enhancing agent to form a solution that is a single liquid continuous phase. The solvent is preferably an aqueous or aqueous-miscible solvent. The solution is then subjected to a phase change, for example, by lowering the temperature of the solution, whereby the dissolved insulin goes through a liquid-solid phase separation to form a suspension of small spherical insulin particles constituting a discontinuous phase while the phase-separation enhancing agent remains in the continuous phase.

Phases:

The Continuous Phase: The method of preparing small spherical insulin particles begins with providing a solution having the active agent and a phase-separation enhancing agent dissolved in a first solvent in a single liquid phase. The solution can be an organic system comprising an organic solvent or a mixture of miscible organic solvents. The solution can also be an aqueous-based solution comprising an aqueous medium or an aqueous-miscible organic solvent or a mixture of aqueous-miscible organic solvents or combinations thereof. The aqueous medium can be water, normal saline, buffered solutions, buffered saline, and the like.

Suitable aqueous-miscible organic solvents include, but are not limited to, N-methyl-2-pyrrolidinone (N-methyl-2-pyrrolidone), 2-pyrrolidinone (2-pyrrolidone), 1,3-dimethyl-2-imidazolidinone (DMI), dimethylsulfoxide, dimethylacetamide, acetic acid, lactic acid, acetone, methyl ethyl ketone, acetonitrile, methanol, ethanol, isopropanol, 3-pentanol, n-propanol, benzyl alcohol, glycerol, tetrahydrofuran (THF), PEG-4, PEG-8, PEG-9, PEG-12, PEG-14, PEG-16, PEG-120, PEG-75, PEG-150, polyethylene glycol esters, PEG-4 dilaurate, PEG-20 dilaurate, PEG-6 isostearate, PEG-8 palmitostearate, PEG-150 palmitostearate, polyethylene glycol sorbitans, PEG-20 sorbitan isostearate, polyethylene glycol monoalkyl ethers, PEG-3 dimethyl ether, PEG-4 dimethyl ether, polypropylene glycol (PPG), polypropylene alginate, PPG-10 butanediol, PPG-10 methyl glucose ether, PPG-20 methyl glucose ether, PPG-15 stearyl ether, propylene glycol dicaprylate/dicaprate, propylene glycol laurate, and glycofurol (tetrahydrofurfuryl alcohol polyethylene glycol ether), or a combination thereof.

The single continuous phase can be prepared by first providing a solution of the phase-separation enhancing agent, which is either soluble in or miscible with the first solvent. This is followed by adding the insulin to the solution. The insulin crystals or other insulin solids may be added directly to the solution, or the insulin crystals or other solids may first be dissolved in a second solvent and then together added to the solution. The second solvent can be the same solvent as the first solvent, or it can be another solvent selected from the list above and which is miscible with the solution. It is preferred that the insulin crystals or other solids can be added to the solution at an ambient temperature or lower or at elevated temperature, provided that the insulin is dissolved in the solution without significant degradation. What is meant by “ambient temperature” is a temperature of around room temperature of about 20° C. to about 40° C.

Phase-separation Enhancing Agent. The phase-separation enhancing agent (PSEA) enhances or induces the liquid-solid phase separation of the active agent from the solution when the solution is subjected to phase separation in which the dissolved active agent molecules amass together to form a suspension of small spherical insulin particles as a discontinuous phase while the phase-separation enhancing agent remains dissolved in the continuous phase. The phase-separation enhancing agent desolubilizes the dissolved active agent when the solution is brought to the phase separation conditions. Suitable phase-separation enhancing agents include, but are not limited to, polymers or mixtures of polymers that are soluble or miscible with the solution. Examples of suitable polymers include linear or branched polymers. These polymers can be water soluble, semi-water soluble, water-miscible, or insoluble.

Types of water-soluble or water-miscible polymers that may be used include carbohydrate-based polymers, polyaliphatic alcohols, poly(vinyl) polymers, polyacrylic acids, polyorganic acids, polyamino acids, co-polymers and block co-polymers (e.g., poloxamers such as Pluronics F127 or F68), terpolymers, polyethers, naturally occurring polymers, polyimides, polymeric surfactants, polyesters, branched polymers, cyclo-polymers, and polyaldehydes.

Preferred polymers are ones that are acceptable as pharmaceutical additives for the intended route of administration of the active agent particles, such as polyethylene glycol (PEG) of various molecular weights, such as PEG 200, PEG 300, PEG 3350, PEG 8000, PEG 10000, PEG 20000, etc. poloxamers such as Pluronics F127 or Pluronics F68, polyvinylpyrrolidone (PVP), hydroxyethylstarch, and other amphiphilic polymers, used alone or in combinations of two or more thereof. The phase-separation enhancing agent can also be a non-polymer such as a mixture of propylene glycol and ethanol.

Liquid-Solid Phase Separation. A liquid-solid phase separation of the dissolved active agent in the solution can be induced by any method known in the art, such as change in temperature, change in pressure, change in pH, change in ionic strength of the solution, change in the concentration of the dissolved active agent, change in the concentration of the phase-separation enhancing agent, change in osmolality of the solution, combinations of two or more of these, and the like.

In a preferred embodiment, the phase change is a temperature-induced phase change by lowering the temperature of the solution such that the active agent spherical particles are formed and suspendably dispersed in the solution. The term “suspendably dispersed in solution” is used herein to denote that the microparticles are in suspension when they are freshly formed, however, they readily settle (witin minutes) to the bottom of vessel (batch process). However the microparticles can easily re-suspended with moderate mechanical force (e.g., shaking). These particles are therefore described herein as being suspendably dispersed in solution.

In this polythermal process, the rate of cooling can be controlled to control the size and shape of the microparticles. Typical cooling rates are controlled from 0.01° C./minute to 600° C./minute, such as 0.05° C./minute, 0.1° C./minute, 0.2° C./minute, 0.5° C./minute, 1° C./minute, 5° C./minute, 10° C./minute, 20° C./minute, 30° C./minute, 40° C./minute, 50° C./minute, 60° C./minute, 70° C./minute, 80° C./minute, 85° C./minute, 90° C./minute, 95° C./minute, 100° C./minute, 150° C./minute, 200° C./minute, 250° C./minute, 300° C./minute, 350° C./minute, 400° C./minute, 450° C./minute, 500° C./minute, or in a range between any such values. The rate of change can be at a constant or linear rate, a non-linear rate, intermittent, or a programmed rate (having multiple phase cycles).

The microparticles can be separated from the PSEA in the solution and purified by washing as will be discussed below.

For solutions in which the freezing point is relatively high, or freezing occurs before the microparticles forms, the solutions can include a freezing point depressing agent, such as propylene glycol, sucrose, ethylene glycol, alcohols (e.g., ethanol, methanol) or aqueous mixtures of freezing-point depression agents to lower the freezing point of the system to allow the phase change in the system without freezing the system. The process can also be carried out such that the temperature is reduced below the freezing point of the system.

Optional Excipients The microparticles of the present application may include one or more excipients in an amount without forming matrices that disperse the active agent. The excipient may imbue the active agent or the particles with additional characteristics such as increased stability of the particles or of the active agents, controlled release of the active agent from the particles, or modified permeation of the active agent through biological tissues. Suitable excipients include, but are not limited to, carbohydrates (e.g., trehalose, sucrose, mannitol), cations (e.g., Zn²⁺, Mg²⁺, Ca²⁺), anions (e.g. SO₄²⁻), amino acids (e.g., glycine), lipids, phospholipids, fatty acids, surfactants, triglycerides, bile acids or their salts (e.g., cholate or its salts, such as sodium cholate; deoxycholic acid or its salts), fatty acid esters, and polymers present in the solution, for example, at levels below their functioning as PSEA's.

Separating and washing the particles. In a preferred embodiment of the present application, the small spherical particles are harvested by separating them from the phase-separation enhancing agent in the solution. In yet another preferred embodiment, the method of separation is by washing the suspendable dispersion containing the small spherical particles with a liquid medium in which the active agent particles are not soluble while the phase-separation enhancing agent is. Some methods of washing may be by diafiltration or by centrifugation. The liquid medium can be an aqueous medium or an organic solvent. For active agent particles with low aqueous solubility, the liquid medium can be an aqueous medium optionally containing agents that reduce the aqueous solubility of the active agent particles, such as divalent cations. For active agents with high aqueous solubility, an organic solvent or an aqueous solvent containing a precipitating agent such as ammonium sulfate may be used.

Examples of suitable organic solvents for use as the liquid medium include, without limitation, those organic solvents specified above as suitable for the continuous phase, and more preferably methylene chloride, chloroform, acetonitrile, ethylacetate, methanol, ethanol, pentane, and the like.

It is also contemplated to use mixtures of any of these solvents. One preferred blend is a 1:1 mixture of methylene chloride and acetone. It is preferred that the liquid medium has a low boiling point for easy removal by, for example, lyophilization, evaporation, or drying.

The liquid medium can also be a supercritical fluid, such as liquid carbon dioxide or a fluid near its supercritical point. Supercritical fluids can be suitable solvents for the phase-separation enhancing agents, particularly some polymers, but are nonsolvents for the spherical protein particles. Supercritical fluids can be used by themselves or with a cosolvent. The following supercritical fluids can be used: liquid CO₂, ethane, or xenon. Potential cosolvents can be acetonitrile, dichloromethane, ethanol, methanol, water, or 2-propanol.

The liquid medium used to separate the small spherical particles from the PSEA described herein, may contain an agent which reduces the solubility of the active agent in the liquid medium. It is desirable that the spherical particles exhibit minimal solubility in the liquid medium to maximize the yield of the spherical particles. For some proteins, such as insulin and human growth hormone, the decrease in solubility can be achieved by the adding of divalent cations, such as Zn²⁺ to the spherical protein particles. Other ions include, but are not limited to, Ca²⁺, Cu²⁺, Fe²⁺, Fe³⁺, and the like.

The solubility of the spherical insulin particles can be sufficiently low to allow diafiltration in an aqueous solution.

The liquid medium may also contain one or more excipients which may imbue the active agent or the particles with additional characteristics such as increased stability of the particles and/or of the active, controlled release of the active agent from the particles, or modified permeation of the active agent through biological tissues as discussed previously.

In another example, the small spherical particles are not separated from the PSEA containing solution.

Aqueous-Based Process. In another preferred embodiment, the solvents of the solution and the liquid washing medium are all aqueous or aqueous-miscible. Examples of suitable aqueous or aqueous-miscible solvents include, but are not limited to, those identified above for the continuous phase. One advantage of using an aqueous-based process is that the media can be buffered and can optionally contain excipients that provide biochemical stabilization to the active agents, such as proteins.

The Active Agent. The active agent includes a pharmaceutically active agent, which can be a therapeutic agent, a diagnostic agent, a cosmetic, a nutritional supplement, or a pesticide.

The therapeutic agent can be a biologic, which includes but is not limited to proteins, polypeptides, carbohydrates, polynucleotides, and nucleic acids. The protein can be an antibody, which can be polyclonal or monoclonal. The therapeutic can be a low molecular weight molecule. In addition, the therapeutic agents can be selected from a variety of known pharmaceuticals such as, but are not limited to: analgesics, anesthetics, analeptics, adrenergic agents, adrenergic blocking agents, adrenolytics, adrenocorticoids, adrenomimetics, anticholinergic agents, anticholinesterases, anticonvulsants, alkylating agents, alkaloids, allosteric inhibitors, anabolic steroids, anorexiants, antacids, antidiarrheals, antidotes, antifolics, antipyretics, antirheumatic agents, psychotherapeutic agents, neural blocking agents, anti-inflammatory agents, antihelmintics, anti-arrhythmic agents, antibiotics, anticoagulants, antidepressants, antidiabetic agents, antiepileptics, antifungals, antihistamines, antihypertensive agents, antimuscarinic agents, antimycobacterial agents, antimalarials, antiseptics, antineoplastic agents, antiprotozoal agents, immunosuppressants, immunostimulants, antithyroid agents, antiviral agents, anxiolytic sedatives, astringents, beta-adrenoceptor blocking agents, contrast media, corticosteroids, cough suppressants, diagnostic agents, diagnostic imaging agents, diuretics, dopaminergics, hemostatics, hematological agents, hemoglobin modifiers, hormones, hypnotics, immunological agents, antihyperlipidemic and other lipid regulating agents, muscarinics, muscle relaxants, parasympathomimetics, parathyroid hormone, calcitonin, prostaglandins, radio-pharmaceuticals, sedatives, sex hormones, anti-allergic agents, stimulants, sympathomimetics, thyroid agents, vasodilators, vaccines, vitamins, and xanthines. Antineoplastic, or anticancer agents, include but are not limited to paclitaxel and derivative compounds, and other antineoplastics selected from the group consisting of alkaloids, antimetabolites, enzyme inhibitors, alkylating agents and antibiotics.

A cosmetic agent is any active ingredient capable of having a cosmetic activity. Examples of these active ingredients can be, inter alia, emollients, humectants, free radical-inhibiting agents, anti-inflammatories, vitamins, depigmenting agents, anti-acne agents, antiseborrhoeics, keratolytics, slimming agents, skin coloring agents and sunscreen agents, and in particular linoleic acid, retinol, retinoic acid, ascorbic acid alkyl esters, polyunsaturated fatty acids, nicotinic esters, tocopherol nicotinate, unsaponifiables of rice, soybean or shea, ceramides, hydroxy acids such as glycolic acid, selenium derivatives, antioxidants, beta-carotene, gamma-orizanol and stearyl glycerate. The cosmetics are commercially available and/or can be prepared by techniques known in the art.

Examples of nutritional supplements include, but are not limited to, proteins, carbohydrates, water-soluble vitamins (e.g., vitamin C, B-complex vitamins, and the like), fat-soluble vitamins (e.g., vitamins A, D, E, K, and the like), and herbal extracts. The nutritional supplements are commercially available and/or can be prepared by techniques known in the art.

The term “pesticide” is understood to encompass herbicides, insecticides, acaricides, nematicides, ectoparasiticides and fungicides. Examples of compound classes include ureas, triazines, triazoles, carbamates, phosphoric acid esters, dinitroanilines, morpholines, acylalanines, pyrethroids, benzilic acid esters, diphenylethers and polycyclic halogenated hydrocarbons. Specific examples of pesticides in each of these classes are listed in Pesticide Manual, 9th Edition, British Crop Protection Council. The pesticides are commercially available and/or can be prepared by techniques known in the art.

In a preferred embodiment, the active agent is a macromolecule, such as a protein, a polypeptide, a carbohydrate, a polynucleotide, a virus, or a nucleic acid. Nucleic acids include DNA, oligonucleotides, antisense oligonucleotides, aptimers, RNA, and SiRNA. The macromolecule can be natural or synthetic. The protein can be an antibody, which can be monoclonal or polyclonal. The protein can also be any known therapeutic proteins isolated from natural sources or produced by synthetic or recombinant methods. Examples of therapeutic proteins include, but are not limited to, proteins of the blood clotting cascade (e.g., Factor VII, Factor VIII, Factor IX, et al.), subtilisin, ovalbumin, alpha-1-antitrypsin (AAT), DNase, superoxide dismutase (SOD), lysozyme, ribonuclease, hyaluronidase, collagenase, growth hormone, erythropoetin, insulin-like growth factors or their analogs, interferons, glatiramer, granulocyte-macrophage colony-stimulating factor, granulocyte colony-stimulating factor, antibodies, PEGylated proteins, glycosylated or hyperglycosylated proteins, desmopressin, LHRH agonists such as: leuprolide, goserelin, nafarelin, buserelin; LHRH antagonists, vasopressin, cyclosporine, calcitonin, parathyroid hormone, parathyroid hormone peptides and insulin. Preferred therapeutic proteins are insulin, alpha-1 antitrypsin, LHRH agonists and growth hormone.

Examples of low molecular weight therapeutic molecules include, but are not limited to, steroids, beta-agonists, anti-microbials, antifungals, taxanes (antimitotic and antimicrotubule agents), amino acids, aliphatic compounds, aromatic compounds, and urea compounds.

In a preferred embodiment, the active agent is a therapeutic agent for treatment of pulmonary disorders. Examples of such agents include, but are not limited to, steroids, beta-agonists, anti-fungals, anti-microbial compounds, bronchial dialators, anti-asthmatic agents, non-steroidal anti-inflammatory agents (NSAIDS), alpha-1-antitrypsin, and agents to treat cystic fibrosis. Examples of steroids include but are not limited to beclomethasone (including beclomethasone dipropionate), fluticasone (including fluticasone propionate), budesonide, estradiol, fludrocortisone, flucinonide, triamcinolone (including triamcinolone acetonide), and flunisolide. Examples of beta-agonists include but are not limited to salmeterol xinafoate, formoterol fumarate, levo-albuterol, bambuterol, and tulobuterol.

Examples of anti-fungal agents include but are not limited to itraconazole, fluconazole, and amphotericin B.

Diagnostic agents include the x-ray imaging agent and contrast media. Examples of x-ray imaging agents include WIN-8883 (ethyl 3,5-diacetamido-2,4,6-triiodobenzoate) also known as the ethyl ester of diatrazoic acid (EEDA), WIN 67722, i.e., (6-ethoxy-6-oxohexyl-3,5-bis(acetamido)-2,4,6-triiodobenzoate; ethyl-2-(3,5-bis(acetamido)-2,4,6-triiodobe-nzoyloxy)butyrate (WIN 16318); ethyl diatrizoxyacetate (WIN 12901); ethyl 2-(3,5-bis(acetamido)-2,4,6-triiodobenzoyloxy)propionate (WIN 16923); N-ethyl 2-(3,5-bis(acetamido)-2,4,6-triiodobenzoyloxy acetamide (WIN 65312); isopropyl 2-(3,5-bis(acetamido)-2,4,6-triiodobenzoyloxy)acetamide (WIN 12855); diethyl 2-(3,5-bis(acetamido)-2,4,6-triiodobenzoyl-oxy malonate (WIN 67721); ethyl 2-(3,5-bis(acetamido)-2,4,6-triiodobenzoyl-oxy)phenylacetate (WIN 67585); propanedioic acid, [[3,5-bis(acetylamino)-2,4,5-triodobenzoyl]oxy]bis(1-methyl)ester (WIN 68165); and benzoic acid, 3,5-bis(acetylamino)-2,4,6-triodo-4-(ethyl-3-ethoxy-2-butenoate) ester (WIN 68209). Preferred contrast agents include those which are expected to disintegrate relatively rapidly under physiological conditions, thus minimizing any particle associated inflammatory response. Disintegration may result from enzymatic hydrolysis, solubilization of carboxylic acids at physiological pH, or other mechanisms. Thus, poorly soluble iodinated carboxylic acids such as iodipamide, diatrizoic acid, and metrizoic acid, along with hydrolytically labile iodinated species such as WIN 67721, WIN 12901, WIN 68165, and WIN 68209 or others may be preferred.

Numerous combinations of active agents may be desired including, for example, a combination of a steroid and a beta-agonist, e.g., fluticasone propionate and salmeterol, budesonide and formeterol, etc.

Examples of carbohydrates are dextrans, hetastarch, cyclodextrins, alginates, chitosans, chondroitins, heparins, others disclosed herein in other contexts, and the like.

The Small Spherical Particles. The small spherical particles of the present application have an average geometric particle size of from about 0.01 μm to about 200 μm, more preferably from 0.1 μm to 10 μm, even more preferably from about 0.5 μm to about 5 μm, and most preferably from about 0.5 μm to about 3 μm, as measured by dynamic light scattering methods (e.g., photocorrelation spectroscopy, laser diffraction, low-angle laser light scattering (LALLS), medium-angle laser light scattering (MALLS)), by light obscuration methods (Coulter analysis method, for example) or by other methods, such as rheology or microscopy (light or electron). Spherical particles for pulmonary delivery have an aerodynamic particle size determined by time of flight measurements (e.g., Aerosizer), Next Generation Impactors, or Andersen Cascade Impactor measurements.

The small spherical particles are substantially spherical. What is meant by “substantially spherical” is that the ratio of the lengths of the longest to the shortest perpendicular axes of the particle cross section is less than or equal to 1.5. Substantially spherical does not require a line of symmetry. Further, the particles may have surface texturing, such as lines or indentations or protuberances that are small in scale when compared to the overall size of the particle and still be substantially spherical. More spherical particles have the ratio of lengths between the longest and shortest axes of less than or equal to 1.33. Most spherical particles have the ratio of lengths between the longest and shortest axes of less than or equal to 1.25. Surface contact is minimized in microspheres that are substantially spherical, which minimizes the undesirable agglomeration of the particles. Many crystals or flakes or irregular particles have flat surfaces that can allow large surface contact areas where agglomeration can occur by ionic or non-ionic interactions. A sphere permits contact over a much smaller area.

The small spherical particles in one exemplary composition have substantially the same particle size, i.e., a monodisperse size distribution, allowing delivery of the active agent to specific areas in the lung, such as the alveoli. In another composition, microparticles having a polydisperse size distribution where there are both relatively big and small particles allow the active agent to be delivered to all areas of the lung rather than portions thereof. What is meant by a “monodisperse size distribution” is a preferred particle size distribution would have a ratio of the volume diameter of the 90^{th} percentile of the small spherical particles to the volume diameter of the 10^{th} percentile less than or equal to 5. More preferably, the particle size distribution would have ratio of the volume diameter of the 90^{th} percentile of the small spherical particles to the volume diameter of the 10^{th} percentile less than or equal to 3. Most preferably, the particle size distribution would have ratio of the volume diameter of the 90^{th} percentile of the small spherical particles to the volume diameter of the 10^{th} percentile less than or equal to 2.

Geometric Standard Deviation (GSD) can also be used to indicate the polydispersity of the particle size distribution. GSD calculations involved determining the effective cutoff diameter (ECD) at the cumulative less than percentages of 15.9% and 84.1%. GSD is equal to the square root of the ratio of the ECD less than 84.17% to ECD less then 15.9%. The small spherical particle composition has a monodisperse size distribution when GSD<2.5, more preferably less than 1.8.

In another example, the active agent in the small spherical particles is semi-crystalline or non-crystalline.

Typically, small spherical particles made by the processes in this application are substantially non-porous and have a density greater than 0.5 g/cm³, more preferably greater than 0.75 g/cm³ and most preferably greater than about 0.85 g/cm³. A preferred range for the density is from about 0.5 to about 2 g/cm³ and more preferably from about 0.75 to about 1.75 g/cm³ and even more preferably from about 0.85 g/cm³ to about 1.5 g/cm³.

The small spherical particles of the present application have high content of the active agent. There is no requirement for a significant quantity of bulking agents or similar excipients that are required by many other methods of preparing particles. For example, insulin small spherical particles have an insulin content of 90% or greater, or 93% or greater, or 95% or greater by weight of the particles. However, bulking agents or excipients may be included in the small spherical particles, but without forming matrices that disperse the active agent, typically less than 20% or 10% or less by weight of the particles. Preferably, the active agent is present greater than 95% by weight of the small spherical particle, and up to 100% by weight. When stating ranges herein, it is meant to include any range or combination of ranges therein.

The active agents incorporated into the small spherical particles disclosed herein retain their biochemical integrity and their biological activity with or without the inclusion of excipients.

In vivo Delivery of the Small Spherical Particles. The small spherical active agent particles in the present application are suitable for in vivo delivery to a subject by a suitable route, such as injectable, topical, oral, rectal, nasal, pulmonary, vaginal, buccal, sublingual, transdermal, transmucosal, otic, intraocular or ocular. The small spherical particles can be delivered as a stable liquid suspension or suspendable dispersion or formulated as a solid dosage form such as dry powders, tablets, caplets, capsules, etc. A preferred delivery route is injectable, which includes intravenous, intramuscular, subcutaneous, intraperitoneal, intrathecal, epidural, intra-arterial, intra-articular and the like. Another preferred route of delivery is pulmonary inhalation, which can be oral or nasal. In this route of delivery, the small spherical particles may be deposited to the deep lung, in the upper respiratory tract, or anywhere in the respiratory tract. The small spherical particles may be delivered as a dry powder by a dry powder inhaler, or they may be formulated and delivered by a metered dose inhaler or a nebulizer.

Drugs intended to function systemically, such as insulin, are desirably deposited in the alveoli, where there is a very large surface area available for drug absorption into the bloodstream. When targeting the drug deposition to certain regions within the lung, the aerodynamic diameter of the particle can be adjusted to an optimal range by manipulating fundamental physical characteristics of the particles such as shape, density, and particle size.

Acceptable respirable fractions of inhaled drug particles in prior art formulations typically require the addition of excipients, either incorporated into each of the particles or as a mixture with the drug particles. For example, improved dispersion of micronized drug particles (about 5 μm) is effected by blending with larger (30-90 μm) particles of inert carrier particles such as trehalose, lactose or maltodextrin. The larger excipient particles improve the powder flow properties, which correlates with an improved pharmacodynamic effect. In a further refinement, the excipients are incorporated directly into the small spherical particles to effect aerosol performance as well as potentially enhancing the stability of protein drugs. Generally, excipients are chosen that have been previously FDA approved for inhalation, such as lactose, or organic molecules endogenous to the lungs, such as albumin and DL-.alpha.-phosphatidylcholine dipalmitoyl (DPPC). Other excipients, such as poly(lactic acid-co-glycolic acid) (PLGA) have been used to engineer particles with desirable physical and chemical characteristics. However, much of the inhalation experience with FDA approved excipients has been with asthma drugs having large aerodynamic particle sizes that desirably deposit in the tracheobronchial region, and which do not appreciably penetrate to the deep lung. For inhaled protein or peptide therapeutics delivered to the deep lung, there is concern that undesirable long-term side effects, such as inflammation and irritation can occur which may be due to an immunological response or caused by excipients when they are delivered to the alveolar region.

In order to minimize potential deleterious side effects of deep lung inhaled therapeutics, it may be advantageous to fabricate particles for inhalation that are substantially constituted by the drug to be delivered. This strategy would minimize alveolar exposure to excipients and reduce the overall mass dose of particles deposited on alveolar surfaces with each dose, possibly minimizing irritation during chronic use of the inhaled therapeutic. Small spherical particles with aerodynamic properties suitable for deep lung deposition that are essentially composed entirely of a therapeutic protein or peptide, such as those disclosed herein, may be particularly useful for isolated studies on the effects of chronic therapeutic dosing on the alveolar membrane of the lung and effective for systemic delivery of the active agent to subjects. The effects of systemic delivery of protein or peptide in the form of small spherical particles by inhalation could then be studied without complicating factors introduced by associated excipients.

The requirements to deliver particles to the deep lung by inhalation are that the particles have a small mean aerodynamic diameter of 0.5-10 micrometers and a monodisperse size distribution. The present application also contemplates mixing together of various batches of small spherical particles having different particle size ranges to yield a composition having polydisperse particle size distribution that is desirable, for example, for local delivery of active agent to the lung tissue. The processes disclosed herein allow the fabrication of small spherical particles with the above characteristics.

There are two principal approaches for forming particles with the herein-described aerodynamic diameters. The first approach is to produce relatively large but very porous (or perforated) microparticles. Since the relationship between the aerodynamic diameter (D_{aerodynamic}) and the geometric diameter (D_{geometric}) is D_{aerodynamic} is equal to D_{geometric} multiplied by the square root of the density of the particles, particles with very low mass density (around 0.1 g/cm³) can exhibit small aerodynamic diameters (0.5 to 3 microns) while possessing relatively high geometric diameters (5 to 10 microns).

An alternative approach is to produce particles with relatively low porosity, in the case of the present application, the particles have a density, set forth in the ranges above, and more generally that is close to 1 g/cm³. Thus, the aerodynamic diameter of such non-porous dense particles is close to their geometric diameter.

The present methods for particle formation set forth above, provides for small spherical particles with or without excipients.

Fabrication of protein small spherical particles from dissolved protein itself with no additives provides options for larger drug payloads in the same volume of solids, increased safety and decreased numbers of required inhalations.

Microencapsulation of Pre-Fabricated Small Spherical Particles. The small spherical particles of the present application encapsulated within matrices of wall-forming materials (which are typically less water-soluble than matrices of excipients) to form microencapsulated particles. The microencapsulation can be accomplished by any process known in the art. In a preferred embodiment, microencapsulation of the small spherical particles of the present application is accomplished by an emulsification/solvent extraction processes as described below. The matrix can impart sustained release properties to the active agent resulting in release rates that persist from minutes to hours, days or weeks according to the desired therapeutic applications. The microencapsulated particles can also produce delayed release formulations of the pre-fabricated small spherical particles. In a preferred embodiment, the pre-fabricated small spherical particles are spherical insulin particles.

In the emulsification/solvent extraction process, emulsification is obtained by mixing two immiscible phases, the continuous phase and the discontinuous phase (which is also known as the dispersed phase), to form an emulsion. In a preferred embodiment, the continuous phase is an aqueous phase (or the water phase) and the discontinuous phase is an organic phase (or the oil phase) to form an oil-in-water (O/W) emulsion. The discontinuous phase may further contain a dispersion of solid particles present either as a fine suspension or as a fine dispersion forming a solid-in-oil (S/O) phase. The organic phase is preferably a water immiscible or a partially water miscible organic solvent. The ratio by weights of the organic phase to the aqueous phase is from about 1:99 to about 99:1, more preferably from 1:99 to about 40:60, and most preferably from about 2:98 to about 1:3, or any range or combination of ranges therein. In a preferred embodiment, the ratio of the organic phase to the aqueous phase is about 1:3. The present application further contemplates utilizing reverse emulsions or water-in-oil emulsion (W/O) where the oil phase forms the continuous phase and water phase forms the discontinuous phase. The present application further contemplates utilizing emulsions having more than two phases such as an oil-in-water-in-oil emulsion (O/W/O) or a water-in-oil-in-water emulsion (W/O/W).

In a preferred embodiment, the process of microencapsulation using the emulsification/solvent extraction process starts with preparing pre-fabricated small spherical particles by the methods described earlier and an organic phase containing the wall-forming material. The pre-fabricated small spherical particles are dispersed in the organic phase of the wall-forming material to form a solid-in-oil (S/O) phase containing a dispersion of the pre-fabricated small spherical particles in the oil phase. In a preferred embodiment, the dispersion is accomplished by homogenizing the mixture of the small spherical particles and the organic phase. An aqueous medium will form the continuous phase. In this case, the emulsion system formed by emulsifying the S/O phase with an aqueous phase is a solid-in-oil-in-water (S/O/W) emulsion system.

The wall-forming material refers to materials capable of forming the structural entity of the matrix individually or in combination. Biodegradable wall-forming materials are preferred, especially for injectable applications. Examples of such materials include but are not limited to the family of poly-lactide/poly-glycolide polymers (PLGA's), polyethylene glycol conjugated PLGA's (PLGA-PEG's), and triglycerides. In the embodiment in which PLGA or PLGA-PEG is used, the PLGA preferably has a ratio of poly-lactide to poly-glycolide of from 100:0 to 0:100, more preferably from about 90:10 to about 15:85, and most preferably about 50:50. In general, the higher the ratio of the poly-glycolide to the poly-lactide in the polymer, the more hydrophilic is the microencapsulated particles resulting in faster hydration and faster degradation. Various molecular weights of PLGA can also be used. In general, for the same ratio of poly-glycolide and poly-lactide in the polymer, the higher the molecular weight of the PLGA, the slower is the release of the active agent, and the wider the distribution of the size of the microencapsulated particles.

The organic solvent in the organic phase (oil phase) of an oil-in-water (O/W) or solid-in-oil-in-water (S/O/W) emulsion can be aqueous immiscible or partially aqueous immiscible. What is meant by the term “water immiscible solvent” is a solvent that forms an interfacial meniscus when combined with an aqueous solution in a 1:1 ratio (O/W). Suitable water immiscible solvents include, but are not limited to, substituted or unsubstituted, linear, branched or cyclic alkanes with a carbon number of 5 or higher, substituted or unsubstituted, linear, branched or cyclic alkenes with a carbon number of 5 or higher, substituted or unsubstituted, linear, branched or cyclic alkynes with a carbon number of 5 or higher; aromatic hydrocarbons completely or partially halogenated hydrocarbons, ethers, esters, ketones, mono-, di- or tri-glycerides, native oils, alcohols, aldehydes, acids, amines, linear or cyclic silicones, hexamethyldisiloxane, or any combination of these solvents. Halogenated solvents include, but are not limited to carbon tetrachloride, methylene chloride, chloroform, tetrachloroethylene, trichloroethylene, trichloroethane, hydrofluorocarbons, chlorinated benzene (mono, di, tri), trichlorofluoromethane. Particularly suitable solvents are methylene chloride, chloroform, diethyl ether, toluene, xylene and ethyl acetate. What is meant by “partially water miscible solvents” is a solvent that is water immiscible at one concentration, and water miscible at another lower concentration. These solvents are of limited water miscibility and capable of spontaneous emulsion formation. Examples of partially water miscible solvents are tetrahydrofuran (THF), propylene carbonate, benzyl alcohol, and ethyl acetate.

A surface active compound can be added, for example, to increase the wetting properties of the organic phase. The surface active compound can be added before the emulsification process to the aqueous phase, to the organic phase, to both the aqueous medium and the organic solution, or after the emulsification process to the emulsion. The use of a surface active compound can reduce the number of unencapsulated or partially encapsulated small spherical particles, resulting in reduction of the initial burst of the active agent during the release. The surface active compound can be added to the organic phase, or to the aqueous phase, or to both the organic phase and the aqueous phase, depending on the solubility of the compound.

What is meant by the term “surface active compounds” is a compound such as an anionic surfactant, a cationic surfactant, a zwitterionic surfactant, a nonionic surfactant or a biological surface active molecule. The surface active compound should be present in an amount by weight of the aqueous phase or the organic phase or the emulsion, whatever the case may be, from less than about 0.01% to about 30%, more preferably from about 0.01% to about 10%, or any range or combination of ranges therein.

Suitable anionic surfactants include but are not limited to: potassium laurate, sodium lauryl sulfate, sodium dodecylsulfate, alkyl polyoxyethylene sulfates, sodium alginate, dioctyl sodium sulfosuccinate, phosphatidyl choline, phosphatidyl glycerol, phosphatidyl inosine, phosphatidylserine, phosphatidic acid and their salts, glyceryl esters, sodium carboxymethylcellulose, cholic acid and other bile acids (e.g., cholic acid, deoxycholic acid, glycocholic acid, taurocholic acid, glycodeoxycholic acid) and salts thereof (e.g., sodium deoxycholate, etc.).

Suitable cationic surfactants include, but are not limited to, quaternary ammonium compounds, such as benzalkonium chloride, cetyltrimethylammonium bromide, lauryldimethylbenzylammonium chloride, acyl carnitine hydrochlorides, or alkyl pyridinium halides. As anionic surfactants, phospholipids may be used. Suitable phospholipids include, for example phosphatidylcholine, phosphatidylethanolamine, phosphatidylserine, phosphatidyl inositol, phosphatidylglycerol, phosphatidic acid, lysophospholipids, egg or soybean phospholipid or a combination thereof. The phospholipid may be salted or desalted, hydrogenated or partially hydrogenated or natural, semisynthetic or synthetic.

Suitable nonionic surfactants include: polyoxyethylene fatty alcohol ethers (Macrogol and Brij), polyoxyethylene sorbitan fatty acid esters (Polysorbates), polyoxyethylene fatty acid esters (Myrj), sorbitan esters (Span), glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxomers), polaxamines, polyvinyl alcohol, polyvinylpyrrolidone, and polysaccharides (including starch and starch derivatives such as hydroxyethylstarch (HES), methylcellulose, hydroxycellulose, hydroxy propylcellulose, hydroxy propylmethylcellulose, and noncrystalline cellulose). In one example, the nonionic surfactant is a polyoxyethylene and polyoxypropylene copolymer and preferably a block copolymer of propylene glycol and ethylene glycol. Such polymers are sold under the tradename POLOXAMER also sometimes referred to as PLURONIC®, and sold by several suppliers including Spectrum Chemical and Ruger. Among polyoxyethylene fatty acid esters is included those having short alkyl chains. One example of such a surfactant is SOLUTOL® HS 15, polyethylene-660-hydroxystearate, manufactured by BASF Aktiengesellschaft.

Surface active biological molecules include such molecules as albumin, casein, heparin, hirudin, hetastarch or other appropriate biocompatible agents.

In a preferred example, the aqueous phase includes a protein as the surface active compound. A preferred protein is albumin. The protein may also function as an excipient. In embodiments in which protein is not the surface active compound, other excipients may be included in the emulsion, added either before or after the emulsification process. Suitable excipients include, but are not limited to, saccharides, disaccharides, and sugar alcohols. A preferred disaccharide is sucrose, and a preferred sugar alcohol is mannitol.

In addition, use of channeling agents, such as polyethylelne glycol (PEG), can increase the water permeation rate of the final product, which results in modification of the initial release kinetics of the active agent from the matrix as well as degradation rate of the matrix and degradation-dependent release kinetics by modifying the hydration rate. Using PEG as the channeling agent during encapsulation can be advantageous in terms of eliminating parts of the washing process during fabrication of the small spherical particles in which PEG is used as the phase-separation enhancing agent. In addition, varying pH of the continuous phase through use of buffers can significantly increase the wetting process between the particle surface and the organic phase, hence, results in significant reduction of the initial burst of the encapsulated therapeutic agent from the matrix of the microencapsulated particles. The properties of the continuous phase can also be modified, for example, by increasing its salinity by adding a salt such as NaCl, to reduce miscibility of the two phases.

After dispersing the small spherical particles in the organic phase (oil phase), the continuous phase of the aqueous medium (water phase) is then vigorously mixed, for example by homogenization or sonication, with the discontinuous phase of the organic phase to form an emulsion containing emulsified droplets of embryonic microencapsulated particles. The continuous aqueous phase can be saturated with the organic solvent used in the organic phase prior to mixing of the aqueous phase and the organic phase, in order to minimize rapid extraction of the organic solvent from the emulsified droplets. The emulsification process can be performed at any temperature in which the mixture can maintain its liquid properties. The emulsion stability is a function of the concentration of the surface active compound in the organic phase or in the aqueous phase, or in the emulsion if the surface active compound is added to the emulsion after the emulsification process. This is one of the factors that determine droplet size of the emulsion system (embryonic microencapsulated particles) and the size and size distribution of the microencapsulated particles. Other factors affecting the size distribution of microencapsulated particles are viscosity of the continuous phase, viscosity of the discontinous phase, shear forces during emulsification, type and concentration of surface active compound, and the Oil/Water ratio.

After the emulsification, the emulsion is then transferred into a hardening medium. The hardening medium extracts the solvent in the discontinous phase from the embryonic microencapsulated particles, resulting in formation of solid microencapsulated particles having a solid polymeric matrix around the pre-fabricated small spherical particles within the vicinity of the emulsified droplets. In the embodiment of an O/W or S/O/W system, the hardening medium is an aqueous medium, which may contain surface active compounds, or thickening agents, or other excipients. The microencapsulated particles are preferably spherical and have a particle size of from about 0.6 to about 300 μm, and more preferably from about 0.8 to about 60 μm. Additionally, the microencapsulated particles preferably have a narrow distribution of particle size. To reduce the extraction time of the discontinuous phase, heat or reduced pressure can be applied to the hardening medium. The extraction rate of discontinuous phase from the embryonic microencapsulated particles is an important factor in the degree of porosity in the final solid microencapsulated particles, since rapid removal, e.g., by evaporation (boiling effect), of the discontinuous phase results in destruction of the continuity of the matrix.

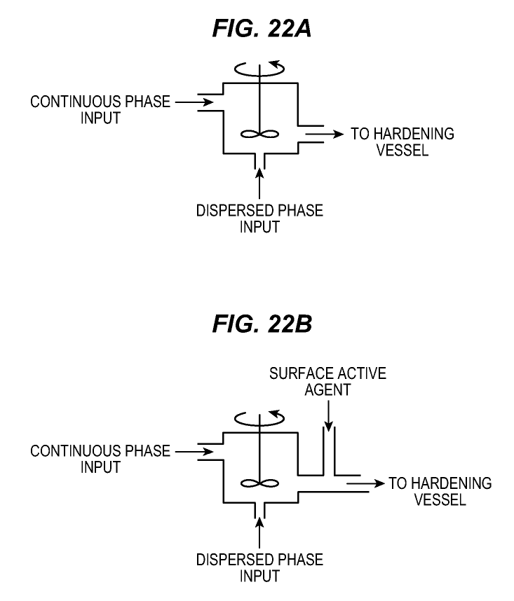

In a preferred embodiment, the emulsification process is performed in a continuous fashion instead of a batch process. FIG. 22 depicts the design of the continuous emulsification reactor.

In another preferred embodiment, the hardened wall-forming polymeric matrices, encapsulating the small spherical particles of the active agent, are further harvested by centrifugation and/or filtration (including diafiltration), and washed with water. The remaining liquid phases can further be removed by a process such as lyophilization or evaporation.

Insulin Formulations for Pulmonary Delivery: A Preferred Exemplary Embodiment

Insulin or an insulin analog, including those that are naturally occurring, synthetic, semi-synthetic, and recombinant, is a particularly preferred protein for use in accordance with the methods and compositions of the present application. As used herein, “insulin”, refers to mammalian insulin and solids thereof (e.g., sodium insulin, zinc insulin), such as bovine, porcine or human insulin, whose sequences and structures are known in the art. The amino acid sequence and spatial structure of human insulin are well-known. Human insulin is comprised of a twenty-one amino acid A-chain and a thirty amino acid B-chain which are cross-linked by disulfide bonds. A properly cross-linked human insulin contains three disulfide bridges: one between position 7 of the A-chain and position 7 of the B-chain, a second between position 20 of the A-chain and position 19 of the B-chain, and a third between positions 6 and 11 of the A-chain.

The term “insulin analog” means proteins that have an A-chain and a B-chain that have substantially the same amino acid sequences as the A-chain and B-chain of human insulin, respectively, but differ from the A-chain and B-chain of human insulin by having one or more amino acid deletions, one or more amino acid replacements, and/or one or more amino acid additions that do not destroy the insulin activity of the insulin analog.

One type of insulin analog, “monomeric insulin analog” is well known in the art. These reportedly are fast-acting analogs of human insulin, including, for example, monomeric insulin analogs wherein: a) the amino acid residue at position B28 is substituted with Asp, Lys, Leu, Val, or Ala, and the amino acid residue at position B29 is Lys or Pro; b) the amino acid residues at positions B28, B29, and B30 are deleted; or c) the amino acid residue at position B27 is deleted. A preferred monomeric insulin analog is ASpB B²⁸. An even more preferred monomeric insulin analog is Lys^{B28} Pro^{B29}.

Monomeric insulin analogs are disclosed in Chance, et al., U.S. Pat. No. 5,514,646; Chance, et al., U.S. patent application Ser. No. 08/255,297; Brems, et al., Protein Engineering, 5:527-533 (1992); Brange, et al., EPO Publication No. 214,826 (published Mar. 18, 1987); and Brange, et al., Current Opinion in Structural Biology, 1:934-940 (1991). These disclosures are expressly incorporated herein by reference for describing monomeric insulin analogs.

Insulin analogs may also have replacements of the amidated amino acids with acidic forms. For example, Asn may be replaced with Asp or Glu. Likewise, Gln may be replaced with Asp or Glu. In particular, Asn(A18), Asn(A21), or Asp(B3), or any combination of those residues, may be replaced by Asp or Glu. Also, Gln(A15) or Gln(B4), or both, may be replaced by either Asp or Glu.

Preferably, the insulin microspheres comprise at least about 90% insulin by weight of the microspheres, at least about 91%, at least about 92%, at least about 93%, at least about 94%, at least about 95%, at least about 96%, at least about 97%, at least about 98% insulin by weight, or at least about 99%, and up to 100%. In especially preferred embodiments, the insulin released from the insulin microsphere has a structure (e.g., chemical and/or conformational) and/or function (e.g., bioactivity) that is indistinguishable from the starting insulin dissolved in the solution, examples of such dissolved insulin have been discussed above.

Molecules, distinct from the proteins of which the microspheres are composed, may be attached to the outer surface of the microspheres by methods known to those skilled in the art to “coat” or “decorate” the microspheres. The microspheres can have a molecule attached to their outer surface. These molecules are attached for purposes such as to facilitate targeting, enhance receptor mediation, and provide escape from endocytosis or destruction, and to alter their release kinetics. For example, biomolecules such as phospholipids may be attached to the surface of the microsphere to prevent degradation in circulation and/or to promote or inhibit interaction with biological membranes, endocytosis by endosomes; receptors, antibodies or hormones may be attached to the surface to promote or facilitate targeting of the microsphere to the desired organ, tissue or cells of the body; and polysaccharides, such as glucans, or other polymers, such as polyvinyl pyrrolidone and PEG, may be attached to the outer surface of the microsphere to enhance or to avoid uptake by macrophages.

In addition, one or more cleavable, erodable or soluble molecules may be attached to the outer surface of or within the microspheres. The cleavable molecules are designed so that the microspheres are first targeted to a predetermined site under appropriate biological conditions and then, upon exposure to a change in the biological conditions, such as a pH change, the molecules are cleaved causing release of the microsphere from the target site. In this way, microspheres are attached to or taken up by cells due to the presence of the molecules attached to the surface of the microspheres. When the molecule is cleaved, the microspheres remain in the desired location, such as within the cytoplasm or nucleus of a cell, and are free to release the proteins of which the microspheres are composed. This is particularly useful for drug delivery, wherein the microspheres contain a drug that is targeted to a specific site requiring treatment, and the drug can be slowly released at that site.

Additionally, the insulin microspheres can be covalently or non-covalently coated with compounds such as fatty acids, lipids, or polymers. The coating may be applied to the microspheres by immersion in the solubilized coating substance, spraying the microspheres with the substance or other methods well known to those skilled in the art.

Exemplary pulmonary compositions are prepared by contacting the insulin microparticles (e.g., insulin microspheres) with a propellant (e.g., a hydrofluoroalkane propellant) to form a suspension and, thereafter, agitating the suspension for a time sufficient to suspend the microparticles in the propellant. Preferably, the compositions are characterized in that the insulin microspheres remain in suspension a minimum of 10 seconds to 10 minutes, preferably, at least 1 to 10 hours, and more preferably, at least 1 to 7 days following agitation.

In preferred embodiments, the pulmonary compositions have a density ratio of ρ_{microparticle} to ρₚᵣₒₚₑₗₗₐₙₜ in the range of 0.05 to 30 and, more preferably, in the range of 0.5 to 3.0. The density ratio is described in more detail below. The embodiments optionally contain a surfactant. Preferably, the propellant is an HFA (hydrofluoroalkane) propellant such as HFA P134a, HFA P227, or a blend of these or other propellants.

Although it is preferred not to include a surfactant in the pulmonary formulations disclosed herein, a surfactant can be added if desired. As used herein, a surfactant is a term of art that refers to an agent which preferentially adsorbs to an interface between two immiscible phases, such as the interface between water and an organic polymer solution, a water/air interface, an organic solvent/air interface, or microparticle/propellant interface.

Surfactants generally possess a hydrophilic moiety and a lipophilic moiety, such that, upon absorbing to microspheres, they tend to present moieties to the external environment that do not attract similarly-coated particles, thus reducing particle agglomeration. Surfactants may also promote absorption of a therapeutic or diagnostic agent and increase bioavailability of the agent.

Synthetic or naturally occurring surfactants known in the art, include phosphoglycerides. Exemplary phosphoglycerides include phosphatidylcholines, such as the naturally occurring surfactant, L-α phosphatidylcholine dipalmitoyl (“DPPC”). The use of surfactants endogenous to the lung may avoid the need for the use of non-physiologic surfactants. Other exemplary surfactants include diphosphatidyl glycerol (DPPG); sodium dodecyl sulfate (SDS), polyethylene glycol (PEG) and its derivatives; polyvinylpyrrolidone (PVP) and its derivatives; polyoxyethylene-9-lauryl ether; a surface active fatty acid, such as palmitic acid or oleic acid; sorbitan trioleate (Span 85); glycocholate; surfactin; poloxamers; sorbitan fatty acid esters such as sorbitan trioleate; tyloxapol and a phospholipid; and alkylated sugars such as octyl glucoside.

One method for preparing a pulmonary preparation of insulin microparticles involves: 1) selecting a propellant, such as a hydrofluoroalkane propellant having a known density, ρₚᵣₒₚₑₗₗₐₙₜ (e.g., ρ_{hydrofluoroalkane}); 2) selecting an insulin microparticle (e.g., insulin microsphere) having a microparticle density ρ_{microparticle} (e.g., ρ_{microsphere}) such that the ratio of ρ_{microparticle} to ρₚᵣₒₚₑₗₗₐₙₜ is in the range of 0.05 to 30 and, more preferably, in the range of 0.5 to 3.0; and 3) contacting a plurality of the microspheres with the propellant to form the pulmonary preparation. Preferably, the propellant is an HFA propellant such as HFA P134a, HFA P227, or a blend of these propellants. In these and other embodiments, the composition preferably does not include a surfactant.

As used herein, the term “ρₚᵣₒₚₑₗₗₐₙₜ” refers to the density of the propellant. In general, such densities are published for these commercially available agents. Similarly, the phrase, “microsphere density, ρ_{microsphere}”, refers to the density of the microspheres. Microsphere density values are published for commercially available microspheres and/or can be determined in accordance with standard methods known to those of ordinary skill in the art. Thus, the density of the microspheres is selected as discussed above to have a ratio which falls within the above-prescribed range. Preferably, the hydrofluoroalkane propellant is an HFA ρₚᵣₒₚₑₗₗₐₙₜ such as HFA P134a, HFA P227, or a blend of these propellants. In certain preferred embodiments, the composition does not include a surfactant.

A method of administering small spherical insulin particle compositions to the pulmonary system of a subject is provided. The method involves administering to the respiratory tract of a subject in need of treatment, an effective amount of a composition to treat the condition.

In a preferred embodiment, spherical insulin particle is administered by inhalation in a dose effective manner to increase circulating insulin protein levels and/or to lower circulating glucose levels. Such administration can be effective for treating disorders such as diabetes or hyperglycemia. Achieving effective doses of insulin requires administration of an inhaled dose of more than about 0.5 μg/kg to about 500 μg/kg insulin, preferably about 3 μg/kg to about 50 μg/kg, and most preferably about 7 μg/kg to about 25 μg/kg. A therapeutically effective amount can be determined by a knowledgeable practitioner, who will take into account factors including insulin level, blood glucose levels, the physical condition of the patient, the patient's pulmonary status, or the like.

Spherical insulin particle is delivered by inhalation to achieve either or both of rapid dissolution and absorption or slow absorption by sustained release of this protein. Administration by inhalation can result in pharmacokinetics comparable or superior to subcutaneous administration of insulin. Inhalation of spherical insulin particles disclosed herein leads to a rapid rise in the level of circulating insulin followed by a rapid fall in blood glucose levels. Different inhalation devices typically provide similar pharmacokinetics when similar particle sizes and similar levels of lung deposition are compared.

Spherical insulin particles can be delivered by any of a variety of inhalation devices known in the art for administration of a therapeutic agent by inhalation. These devices include metered dose inhalers, nebulizers, dry powder generators, sprayers, and the like. There are several desirable features of an inhalation device for administering spherical insulin particles. For example, delivery by the inhalation device is advantageously reliable, reproducible, and accurate. The inhalation device should deliver small microspheres, e.g. less than about 10 μm, preferably about 0.2-5 μm, for good respirability. Some specific examples of commercially available inhalation devices are Turbuhaler™ (Astra, Wilmington, Del.), Rotahaler® (Glaxo, Research Triangle Park, N.C.), Diskus® (Glaxo, Research Triangle Park, N.C.), Spiros™ inhaler (Dura, San Diego, Calif.), devices marketed by Inhale Therapeutics (San Carlos, Calif.), AERx™ (Aradigm, Hayward, Calif.), the Ultravent® nebulizer (Mallinckrodt, Hazelwood, Mo.), the Acorn II® nebulizer (Marquest Medical Products, Totowa, N.J.), the Ventolin® metered dose inhaler (Glaxo, Research Triangle Park, N.C.), the Spinhaler® powder inhaler (Aventis, Bridgewater, N.J.), and metered dose inhalers supplied by Bespak (London, UK); 3M (Minneapolis, Minn.); Valois (France), or the like.

The insulin microsphere in the formulation delivered by the inhalation device affects the ability of the protein to make its way into the lungs, and preferably into the lower airways or alveoli for systemic administration. Preferably, the insulin microspheres are formulated so that at least about 10% to 40% of the insulin delivered is deposited in the lung, preferably about 40% to about 50%, or more, and, more preferably, 70% to 80%, or more. It is known that the maximum efficiency of pulmonary deposition for mouth breathing humans is obtained with particles having aerodynamic diameters of about 0.1 μm to about 10 μm. When aerodynamic diameters are above about 5 μm, pulmonary deposition decreases substantially. Thus, microspheres of insulin delivered by inhalation have an aerodynamic diameter preferably less than about 10 μm, more preferably in the range of about 0.1 μm to about 5 μm, and most preferably in the range of about 0.1 μm to about 3 μm. The formulation of insulin microspheres is selected to yield the desired aerodynamic diameter in the chosen inhalation device.

Formulations of insulin for administration by inhalation typically include the insulin microspheres disclosed herein and, optionally, a bulking agent, surfactant, carrier, excipient, another additive, or the like. Additives can be included in the formulation of insulin microspheres, for example, to dilute the microspheres as required for delivery by inhalation, to facilitate processing of the formulation, to provide advantageous properties to the formulation, to facilitate dispersion of the formulation from the inhalation device, to stabilize the formulation (e.g., antioxidants or buffers), to provide taste to the formulation, or the like. The insulin microspheres can be mixed with an additive at a molecular level or the solid formulation can include insulin microspheres mixed with or coated on particles of the additive. Typical additives include mono-, di-, and polysaccharides; sugar alcohols and other polyols, such as, for example, lactose, glucose, raffinose, melezitose, lactitol, maltitol, trehalose, sucrose, mannitol, starch, or combinations thereof; surfactants, such as sorbitols, diphosphatidyl choline, or lecithin; or the like. When used in a formulation, the additive is present in an amount effective for a purpose described above, often at about 0.1% to about 90% by weight of the formulation. Additional agents known in the art for formulation of a protein can be included in the formulation.

Administration of a formulation of insulin microspheres by inhalation is a preferred method for treating diabetes. In the methods of the present application it has been found that the insulin microsphere compositions can be delivered via oral inhalation and do not produce cough or shortness of breath. In addition, the bioavailability of the insulin delivered through the methods and compositions is higher than that seen through other methods. Bioavailabilities as high as 31% were observed, with an average bioavailability of 12%. Without being bound to a particular theory or mechanism of action, it is believed that the aerodynamic properties of the small spherical Insulin particle compositions formulated herein favor a higher percentage of the powder mass reaching the deep lung, the primary site where dissolved insulin is readily absorbed. Moreover, the deposition of the spherical insulin particles into the lungs with pulmonary administration of the compositions was higher than predicted.

A spray including insulin microspheres can be produced by forcing a suspension of insulin microspheres suspended in a propellant or other liquid suspending agents through a nozzle under pressure. The nozzle size and configuration, the applied pressure, and the liquid feed rate can be chosen to achieve the desired output and droplet size using any inhalation device known to those of skill in the art. An electrospray or piezoelectric spray can be produced, for example, by an electric field in connection with a capillary or nozzle feed. Advantageously, insulin microspheres delivered by a sprayer have a particle size less than about 10 μm, preferably in the range of about 0.1 μm to about 5 μm, and most preferably about 0.1 μm to about 3 μm.

Formulations of insulin microspheres suitable for use with a nebulizer typically include an aqueous suspension of the microspheres at a concentration of about 1 mg to about 20 mg of insulin per ml of suspension. The formulation can include agents such as an excipient, a buffer, an isotonicity agent, a preservative, a surfactant, a polymer (e.g., polyethylene glycol), and, a metal ion such as zinc or calcium. The formulation can also include an excipient or agent for stabilization of the microspheres and/or insulin therein, such as a buffer, a reducing agent, a bulk protein, or a carbohydrate. Bulk proteins useful in formulating insulin include albumin, protamine, or the like. Typical carbohydrates useful in formulating insulin include sucrose, mannitol, lactose, trehalose, glucose, or the like. In general, the insulin microsphere formulations do not contain a surfactant because the insulin microspheres do not have a tendency to aggregate.

Insulin microspheres can be administered by a nebulizer, such as a jet nebulizer or an ultrasonic nebulizer. Typically, in a jet nebulizer, a compressed air source is used to create a high-velocity air jet through an orifice. As the gas expands beyond the nozzle, a low-pressure region is created, which draws a suspension of insulin microspheres through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, typically employing a piezoelectric transducer. This energy is transmitted to the formulation of insulin microspheres either directly or through a coupling fluid, creating an aerosol including the insulin microspheres. Advantageously, insulin microspheres delivered by a nebulizer have a particle size less than about 10 μm, preferably in the range of about 0.1 μm to about 5 μm, and most preferably about 0.1 μm to about 3 μm.

Formulations of insulin microspheres suitable for use with a nebulizer, either jet or ultrasonic, typically include insulin microspheres in a suspension at a concentration of about 1 mg to about 20 mg of insulin per ml of suspension. The formulation can include additional agents such as those mentioned above (e.g., excipients, buffers, and so forth).

In a metered dose inhaler (MDI), a propellant, insulin microspheres, and any excipients or other additives are contained in a canister as a mixture including a liquefied compressed gas. Actuation of the metering valve releases the mixture as an aerosol, preferably containing microspheres in the size range of less than about 10 μm, preferably about 0.1 μm to about 5 μm, and most preferably about 0.1 μm to about 3 μm. The desired microsphere size can be obtained by employing a formulation of insulin produced by the methods disclosed herein. Preferred metered dose inhalers include those manufactured by Bespak, Valois, 3M or Glaxo and employing a propellant.

Formulations of insulin microspheres for use with a metered-dose inhaler device will generally include the microspheres as a suspension in a non-aqueous medium, for example, suspended in a propellant. In general, a surfactant is not needed because the insulin microspheres disclosed herein have a consistent size and do not have a tendency to aggregate. The propellant may be any conventional material employed for this purpose, such as a chlorofluorocarbon, including trichlorofluoromethane, dichlorodifluoromethane, dichlorotetrafluoroethanol; and a hydrofluoroalkane, including HFA P134a (1,1,1,2-tetrafluoroethane), HFA P227 (1,1,1,2,3,3,3-heptafluoropropane-227); or any other propellant that is useful. Preferably the propellant is a hydrofluoroalkane. Additional agents known in the art for formulation of a protein such as insulin can also be included in the formulation.

Dry powder dispensers for use with the small spherical particles disclosed herein include a unit dose dry powder inhaler (UDPI), a reservoir dry powder inhaler (RDPI), and a multi-dose dry powder inhaler (MDPI). In the UDPI, each powder-filled unit of the carrying member contains or otherwise carries a single defined dose of the powder. In the RDPI, a reservoir contains or otherwise carries multiple (un-metered) doses of the powder, and includes means for metering a dose portion of the powder from the reservoir upon actuation. The metering means include, for example, a metering cup, which is movable from a first position where the cup is filled with powder from the reservoir to a second position where the metered dose of powder is dispensed. In the MDPI, each powder-filled unit of the carrying member contains or otherwise carries multiple, defines doses of the powder.

DPIs are the first breath-actuated inhalers that improved patient compliance. They provide ease in coordinating actuation and inhalation, making them superior alternatives to MDI in inhalation therapy. Common features of DPI devices include a means to open (e.g., puncture, cut, pierce, or peal) the filled carrying member, a space for placing the filled carrying member (e.g., slot or chamber with room for movement, or tight-fitting cup), a mouthpiece through which the subject inhales, and optionally a grid to prevent the carrying member from being inhaled. Simple DPIs have been shown to be effective in the delivery of the active agent to the sites of action and/or absorption in the lungs. Non-limiting examples of single DPI devices include the puncturing variety, such as Spinhaler® (Fisons, UK), Cyclohaler™ (Pharmachemie, Haarlem, the Netherlands), Handihaler® (Boehringer Ingelheim), Floradil® DPI (Novatis), the cutting variety, Flowcaps® (Hovione) and Eclipse™ (Aventis), as well as the compression variety, such as Rotohaler® (GSK).

Formulations of small spherical particles suitable for use with a DPI typically include flowable and dispersible powders, such as those disclosed herein containing the spherical insulin particles. The powder formulations are loaded into one or more carrying members designed to be placed in a housing of the DPI device with a mechanism to allow the powder exit the carrying member upon actuation of the DPI device. Non-limiting examples of the carrying member include capsules, blisters, cartridges, or a substrate onto which the powder is applied by any suitable process including printing, painting and vacuum occlusion, provided singly or in a magazine or cartridge or array or other packages (e.g., elongated form like strips or tapes, or curved form like circles on a disc-shaped substrate) of discrete multiples. Preferred carrying members can be punctured, cut, pierced, peeled, or otherwise opened cleanly without shedding pieces, with the opening remain open without reclosing or obstruction, empty the powder cleanly with minimal retention due to adhesion and triboelectrification, have minimal interaction with the filled powder, withstand changes in moisture level (especially having low moisture levels of 10% to 5% or even to 1% or less) and/or serve as a moisture barrier for the filled powder, deter microbiological infiltrations and proliferations (e.g., by irradiation with UV during molding), and have a tight weight tolerance (e.g., in single-digit milligrams) and/or be lighter than the filled powder to reduce variations in filling.

Non-limiting examples of capsules, typically formed by molding, include 2-piece (with a body and a cap) hard capsules capable of being punctured or cut to release their contents. The capsules can be transparent, translucent, opaque, or colored. Non-limiting examples of blisters, typically formed by thermoforming having a cavity made of either viscous, flexible, transparent plastics or brittle, film-type material (e.g., foil paper) over which a brittle, film-type material (e.g., foil paper) is laminated, include those with foil sealed over foil cavity and foil sealed over plastic cavity. Non-limiting natural or synthetic materials include gelatin blends (typically with water and colorants), celluloses, cellulose-based polymers (e.g., cellulose acetate phthalate (CAP), hydroxypropyl methylcellulose (HPMC, hypromellose in short), esterified HPMC, hydroxypropyl methylcellulose phthalate (HPMCP), hydroxypropyl methylcellulose acetate succinate (HPMCAS)), vinyl polymers (e.g., polyvinyl acetate phthalate (PVAP), Aclar®, PVC®), acrylic polymers (e.g., copolymers of methyl methacrylate, ethyl acrylate, methyl acrylate, and/or methacrylic acid), hypromellose. Low moisture content carrying members include those with a moisture content of 5-8% or 4-6% or less, such as those made of HPMC and derivatives thereof (e.g., Vcaps™ by Capsugel and Quali-V® by Shionogi Qualicaps).

One of ordinary skill in the art will recognize that the methods of use disclosed herein may be achieved by pulmonary administration of insulin microspheres via other suitable devices not described herein.

One method involves dispersing one or more therapeutic doses into a pulmonary delivery device, said therapeutic doses containing a therapeutically effective amount of a insulin microsphere composition disclosed herein. As used herein, a “therapeutically effective amount” refers to that amount of active agent necessary to delay the onset of, inhibit the progression of, or alleviate the particular condition being treated. Generally, a therapeutically effective amount will vary with the subject's age, condition, and sex, as well as the nature and extent of the disease in the subject, all of which can be determined by one of ordinary skill in the art. The dosage may be adjusted by the individual physician or veterinarian, particularly in the event of any complication. A therapeutically effective amount of active agent typically varies from 1 pg/kg to about 1000 mg/kg, preferably from about 1 μg/kg to about 200 mg/kg, and most preferably from about 0.1 mg/kg to about 20 mg/kg, in one or more dose administrations daily, for one or more days, weekly, monthly, every two or three months, and so forth.

The insulin microspheres may be administered alone or in combination with other drug therapies as part of a pharmaceutical composition. Such a pharmaceutical composition may include the insulin microspheres in combination with any standard physiologically and/or pharmaceutically acceptable carriers which are known in the art. The compositions may be sterile and contain a therapeutically effective amount of the microsphere in a unit of weight or volume suitable for administration to a patient. The term “pharmaceutically-acceptable carrier” as used herein means one or more compatible solid or liquid filler, diluents or encapsulating substances which are suitable for administration into a human or other animal. The term “carrier” denotes an organic or inorganic ingredient, natural or synthetic, with which the active ingredient is combined to facilitate the application. The components of the pharmaceutical compositions also are capable of being co-mingled with the molecules disclosed herein, and with each other, in a manner such that there is no interaction which would substantially impair the desired pharmaceutical efficacy. Pharmaceutically acceptable further means a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, desiccants, bulking agents, propellants, acidifying agents, coating agents, solubilizers, and other materials which are well known in the art. Carrier formulations suitable for oral, subcutaneous, intravenous, intramuscular, etc. administrations can be found in Remington's Pharmaceutical Sciences, Mack Publishing Co., Easton, Pa.

Various pharmaceutical compositions are provided herein that include a container containing one or more doses of insulin microspheres. The number of microspheres in the single dose is dependent upon the amount of active agent present in each microsphere and the period of time over which release is desired. Preferably, the single dose is selected to achieve a duration of release of the active agent over a period of 0.1 hours to 96 hours with the desired release profile.

One method involves dispersing one or more therapeutic doses into a package for use with a pulmonary delivery device, said therapeutic insulin doses containing a therapeutically effective amount of a insulin microspheres as disclosed herein.

The package preferably contains between one or two or more, and up to 500 therapeutic doses of the insulin microspheres for treating, for example, diabetes by the release of the active agent in vivo into the lungs of mammal, preferably a human. The number of microspheres present in the single dose is dependent on the type and activity of the active agent. Preferably, a single dose is selected to achieve release over a period of time which has been optimized for treating the particular medical condition.

The package preferably provides instructions for using the container to deliver its contents to a pulmonary delivery device and, optionally, additional instructions for using the inhaler device according to manufacturer's instructions.

The insulin pulmonary pharmaceutical compositions may conveniently be presented in unit dosage form, for example a capsule and may be prepared by any of the methods well-known in the art of pharmacy. All methods may or may not include the step of bringing the microspheres into association with a carrier which constitutes one or more accessory ingredients. The compositions may be prepared by uniformly and intimately bringing the microspheres into association with a liquid carrier, a finely divided solid carrier, or both, and then, if necessary, shaping the product.

### EXAMPLES

### A. Insulin Small Spherical Particles

### Example 1

### General Method of Preparation of Insulin Small Spherical Particles

A solution buffered at pH 5.65 (0.033M sodium acetate buffer) containing 16.67% PEG 3350 was prepared. A concentrated slurry of zinc crystalline insulin was added to this solution while stirring. The insulin concentration in the final solution was 0.83 mg/mL. The solution was heated to about 85 to 90° C. The insulin crystals dissolved completely in this temperature range within five minutes. Insulin small spherical particles started to form at around 60° C. when the temperature of the solution was reduced at a controlled rate. The yield increased as the concentration of PEG increased. This process yields small spherical particles with various size distribution with a mean of 1.4 μm.

The insulin small spherical particles formed were separated from PEG by washing the microspheres via diafiltration under conditions in which the small spherical particles do not dissolve. The insulin small spherical particles were washed out of the suspension using an aqueous solution containing Zn²⁺. The Zn²⁺ ion reduces the solubility of the insulin and prevents dissolution that reduces yield and causes small spherical particle agglomeration.

### Example 2

### Non-Stirred Batch Process for Making Insulin Small Spherical Particles

20.2 mg of zinc crystalline insulin were suspended in 1 mL of deionized water at room temperature. 50 microliters of 0.5N HCl was added to the insulin. 1 mL of deionized water was added to form a 10 mg/mL solution of zinc crystalline insulin. 12.5 g of Polyethylene Glycol 3350 (Sigma) and 12.5 g of Polyvinylpyrrolidone (Sigma) were dissolved in 50 mL of 100 millimolar sodium acetate buffer, pH 5.7. The polymer solution volume was adjusted to 100 mL with the sodium acetate buffer. To 800 microliters of the polymer solution in an eppendorf tube was added 400 microliters of the 10 mg/mL insulin solution. The insulin/polymer solution became cloudy on mixing. A control was prepared using water instead of the polymer solution. The eppendorf tubes were heated in a water bath at 90° C. for 30 minutes without mixing or stirring, then removed and placed on ice for 10 minutes. The insulin/polymer solution was clear upon removal from the 90° C. water bath, but began to cloud as it cooled. The control without the polymer remained clear throughout the experiment. Particles were collected from the insulin/polymer tube by centrifugation, followed by washing twice to remove the polymer. The last suspension in water was lyophilized to obtain a dry powder. SEM analysis of the lyophilized particles from the insulin/polymer tubes showed a uniform distribution of small spherical particles around 1 micrometer in diameter. Coulter light scattering particle size analysis of the particles showed a narrow size distribution with a mean particle size of 1.413 micrometers, 95% confidence limits of 0.941-1.88 micrometers, and a standard deviation of 0.241 micrometers. An insulin control without polymer or wash steps, but otherwise processed and lyophilized in the same manner, showed only flakes (no particles) under the SEM similar in appearance to that typically obtained after lyophilizing proteins.

### Example 3

### The Continuous Flow Through Process for Making Insulin Small Spherical Particles

36.5 mg of insulin was weighed out and suspended in 3 mL of deionized water. 30 μL of 1 N HCl was added to dissolve the insulin. The final volume of the solution was adjusted to 3.65 mL with deionized water. 7.3 mL of PEG/PVP solution (25% PEG/PVP pH 5.6 in 100 mM NaOAc buffer) was then added to the insulin solution to a final total volume of 10.95 mL of insulin solution. The solution was then vortexed to yield a homogenous suspension of insulin and PEG/PVP.

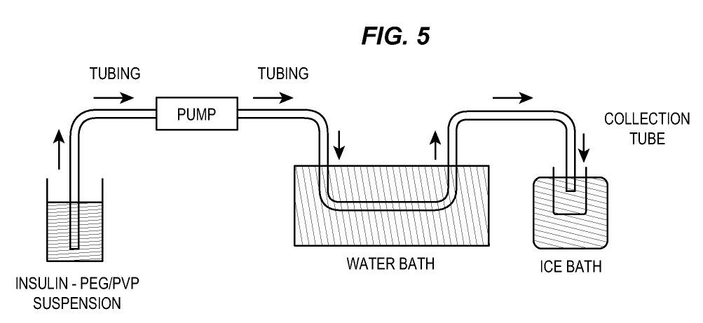

The insulin suspension was connected to a BioRad peristaltic pump running at a speed of 0.4 mL/min through Teflon® tubing (TFE 1/32″ inner diameter flexible tubing). The tubing from the pump was submerged into a water bath maintained at 90° C. before being inserted into a collection tube immersed in ice. Insulin small spherical particles were formed when the temperature of the insulin solution was decreased from about 90° C. in the water bath to about 4° C. in the collection tube in ice. FIG. 5 is a schematic diagram of this process. The total run time for the process was 35 minutes for the 10.95 mL volume. After collecting the small spherical particles, the collection tube was centrifuged at 3000 rpm for 20 minutes in a Beckman J6B centrifuge. A second water wash was completed and the small spherical particle pellets were centrifuged at 2600 rpm for 15 minutes. The final water wash was centrifuged at 1500 rpm for 15 minutes. An aliquot was removed for particle size analysis. The small spherical particles were frozen at −80° C. and lyophilized for 2 days.

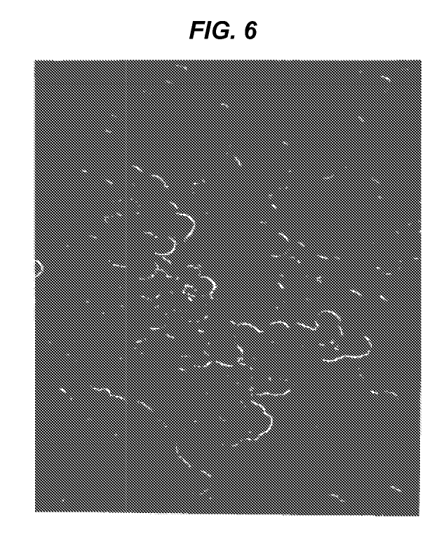

The particle size was determined to be 1.397 μm by volume, 1.119 μm by surface area, and 0.691 μm by number as determined by the Beckman Coulter LS 230 particle counter. The scanning electron micrograph indicated uniform sized and non-agglomerated insulin small spherical particles (FIG. 6).

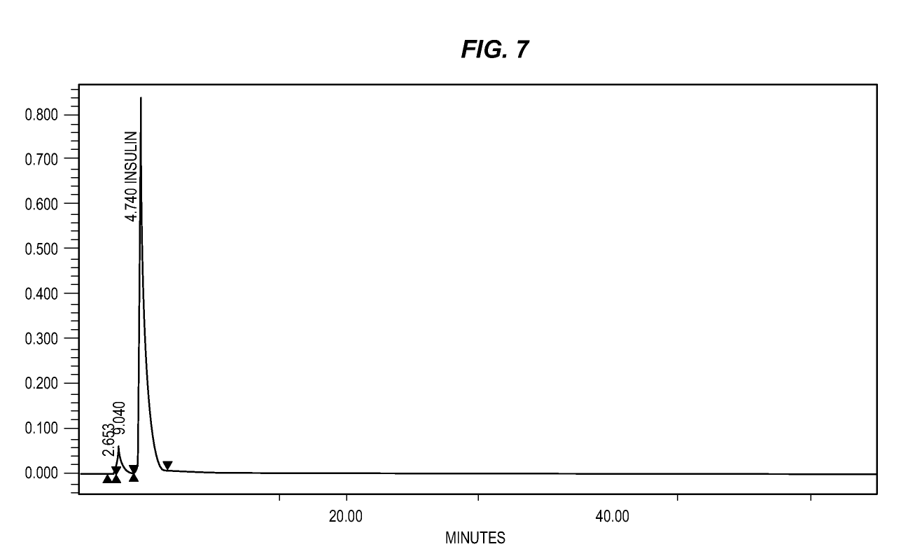

The use of the continuous flow through process where the insulin solution was exposed to 90° C. for a short period of time allowed for the production of small spherical particles. This method yielded a final composition that was 90% protein as determined by high performance liquid chromatography (HPLC) (FIG. 7). HPLC analysis also indicated that the dissolved insulin small spherical particles had an elution time of about 4.74 minutes, not significantly different from that of an insulin standard or the native insulin starting material, indicating that preservation of the biochemical integrity of the insulin after fabrication into the small spherical particles.

### Example 4

### Heat Exchanger Batch Process for Making Insulin Small Spherical Particles

Human zinc crystalline insulin was suspended in a minimal amount of deionized water with sonication to ensure complete dispersion. The insulin suspension was added to a stirred, buffered polymer solution (pH 5.65 at 25° C.) pre-heated to 77° C., so that the final solute concentrations were 0.83% zinc crystalline insulin, 18.5% polyethylene glycol 3350, 0.7% sodium chloride, in a 0.1 M sodium acetate buffer. The initially cloudy mixture cleared within three minutes as the crystalline insulin dissolved. Immediately after clearing, the solution was transferred to a glass, water-jacketed chromatography column that was used as a heat exchanger (column i.d.: 25 mm, length: 600 mm; Ace Glass Incorporated, Vineland, N.J.). The glass column was positioned vertically, and the heat exchange fluid entered the water jacket at the bottom of the column and exited at the top. In order to document the heat exchange properties of the system, thermocouples (Type J, Cole Parmer) were positioned in the center of the insulin formulation liquid at the top and bottom of the column and a cooling temperature profile was obtained during a preliminary trial run. The thermocouples were removed during the six batches conducted for this experiment so as not to introduce a foreign surface variable.

The heat exchanger was pre-heated to 65° C. and the insulin-buffered polymer solution was transferred in such a manner that the solution temperature did not drop below 65° C. and air bubbles were not introduced into the solution. After the clear solution was allowed four minutes to equilibrate to 65° C. in the heat exchanger, the heat exchange fluid was switched from a 65° C. supply to a 15° C. supply. The insulin formulation in the heat exchanger was allowed to equilibrate to 15° C. over a twenty-minute period. The insulin small spherical particles formed as the temperature dropped through 60 to 55° C. resulting in a uniform, stable, creamy white suspension.

The insulin small spherical particles were separated from the polyethylene glycol by diafiltration (A/G Technologies, 750,000 MWCO ultrafiltration cartridge) against five volumes of 0.16% sodium acetate-0.026% zinc chloride buffer, pH 7.0, followed by concentration to one fifth of the original volume. The insulin small spherical particles suspension was further washed by diafiltration against five volumes of deionized water, followed by lyophilization to remove the water. Care was taken to prevent agglomeration of the small spherical particles during diafiltration (from polarization packing of particles on the membrane surface) and during lyophilization (from settling of the small spherical particles prior to freezing). The dried small spherical particles were free flowing and ready for use, with no de-agglomeration or sieving required.

Small Spherical Insulin Particles: The above described process produces uniform size spherical insulin particles from zinc crystalline insulin without added excipients. Small spherical insulin particles prepared by this process have excellent aerodynamic properties as determined by time-of-flight (Aerosizer™) and Andersen Cascade Impactor measurements, with high respirable fractions indicative of deep lung delivery when delivered from a simple, widely used dry powder inhaler (Cyclohaler™). By using insulin as a model protein, we are also able to examine the effect of the process on the chemical integrity of the protein using established U.S.P. methods.

Dry powder insulin small spherical particles were imaged by polarized light microscopy (Leica EPISTAR®, Buffalo, N.Y.) and with a scanning electron microscope (AMRAY 1000, Bedford, Mass.). Particle size analysis was performed using an Aerosizer® Model 3292 Particle Sizing System which included a Model 3230 Aero-Disperser® Dry Powder Disperser for introducing the powder to the instrument (TSI Incorporated, St. Paul, Minn.). Individual particle sizes were confirmed by comparing the Aerosizer results to the electron micrographs.

The chemical integrity of the insulin before and after the process was determined by HPLC according to the USP monograph for Insulin Human (USP 26). The insulin and high molecular weight protein content was measured using an isocratic SEC HPLC method with UV detection at 276 nm. To measure insulin, A-21 desamido insulin and other insulin related substances, the sample was analyzed using a USP gradient reverse-phase HPLC method. The insulin content is measured using UV detection at 214 nm. High molecular weight protein, desamido insulin, and other insulin related substances were assayed to quantitate any chemical degradation caused by the process.

The aerodynamic characteristics of the insulin small spherical particles were examined using the Aerosizer® instrument. Size distribution measurements on insulin dry powder were conducted using the AeroDisperser attachment with low shear force, medium feed rate, and normal deagglomeration. The instruments' software converts time-of-flight data into size and places it into logarithmically spaced ranges. The number of particles detected in each size bin was used for statistical analysis, as well as the total volume of particles detected in each size bin. The volume distribution emphasizes large particles more than the number distribution and, therefore, is more sensitive at detecting agglomerates of non-dispersed particles as well as large particles.

The Andersen Cascade Impactor assembly consisted of a pre-separator, nine stages, eight collection plates, and a backup filter. The stages are numbered −1, −0, 1, 2, 3, 4, 5, 6, and F. Stage −1 is an orifice stage only. Stage F contains the collection plate for Stage 6 and the backup filter. The stainless steel collection plates were coated with a thin layer of food grade silicone to prevent “bounce” of the particles. A sample stream air-flow rate of 60 LPM through the sampler was used for the analysis. An accurately weighed sample size of approximately 10 mg was weighed into each starch capsule (Vendor), with the powder delivered as an aerosol from the Cyclohaler in four seconds. The amount of insulin powder deposited on each plate was determined by reversed phase HPLC detection at 214 nm according to the USP 26 assay for human insulin.

The mass median aerodynamic diameter (MMAD) was calculated by Sigma Plot software using a probit fit of the cumulative less than mass percent versus the effective cutoff diameter (ECD). Emitted dose (ED) was determined as the total observed mass of insulin deposited into the cascade Impactor. This is expressed as a percentage of the mass of the insulin small spherical particles loaded into the Cyclohaler capsule.

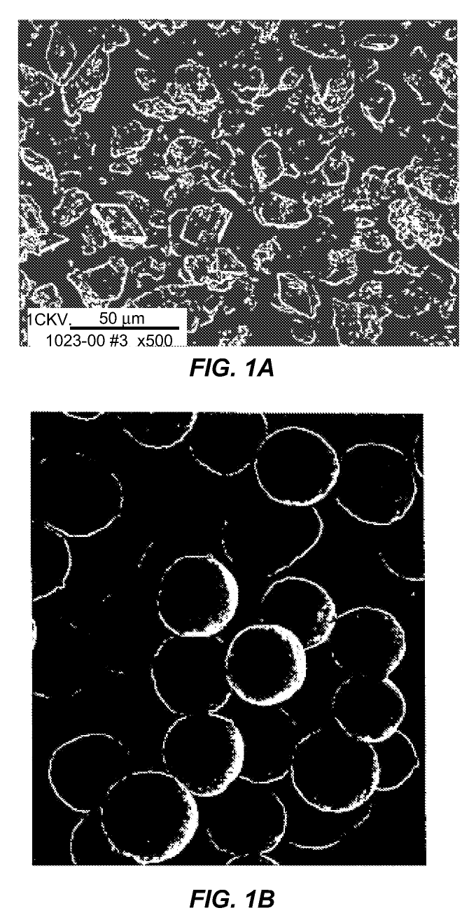

The results demonstrate that careful control of process parameters in conjunction with a phase change formulation can produce: 1) predominantly spherical insulin particles with a diameter of about 2 μm; 2) a monodisperse size distribution; 3) and reproducible aerodynamic properties from batch to batch; and 4) small spherical particles composed of 95% or more human insulin excluding residual moisture. We determined that the solubility of the zinc crystalline insulin could be controlled by solution temperature, pH, polymer concentration, and ionic strength. We also found that controlling the cooling rate allowed the formation of predominantly spherical insulin particles within a narrow size range.

Whereas an SEM of the starting human zinc crystalline insulin raw material shows non-homogenous size and crystalline shapes with particle sizes of approximately 5 to 40 μm, SEM pictures taken of one of the batches from this Example show the spherical shape and uniform size of the insulin small spherical particles (FIG. 1b). The particle shape and size illustrated by the SEM is representative of the other five batches prepared for this Example.

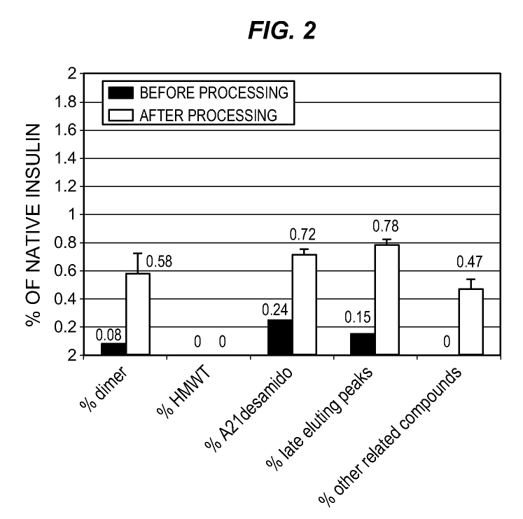

Following separation from the buffered polymer by diafiltration washing and lyophilization from a deionized water suspension, the dry powder insulin small spherical particles were relatively free flowing and easily weighed and handled. The insulin small spherical particles moisture content ranged from 2.1 to 4.4% moisture, compared to 12% for the starting zinc crystalline insulin raw material. Chemical analysis of the insulin small spherical particles by HPLC indicated very little chemical degradation of insulin due to the process (FIG. 2), with no increase in high molecular weight compounds. Although there was an increase (over the starting insulin raw material) in % dimer, % A21 desamido insulin, % late eluting peaks, and % other compounds, the results for all six batches were within USP limits. Retention of insulin potency was 28.3 to 29.9 IU/mg, compared to 28.7 IU/mg for the starting raw material. Residual levels of the polymer used in the process (polyethylene glycol) were below 0.13% to non-detectable, indicating that the polymer is not a significant component of the insulin small spherical particles.

Inter-Batch Reproducibility of Aerodynamic Properties for Insulin Small Spherical Particles

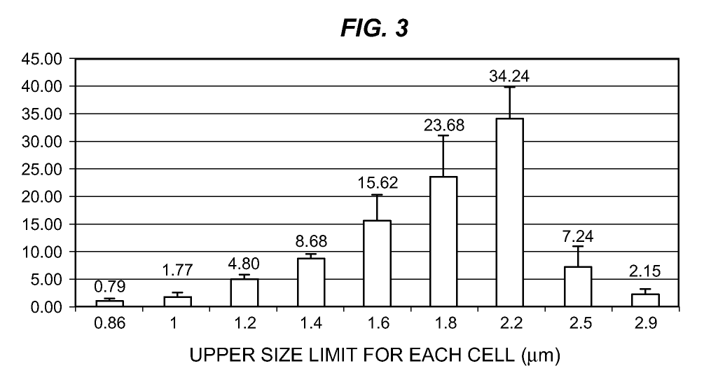

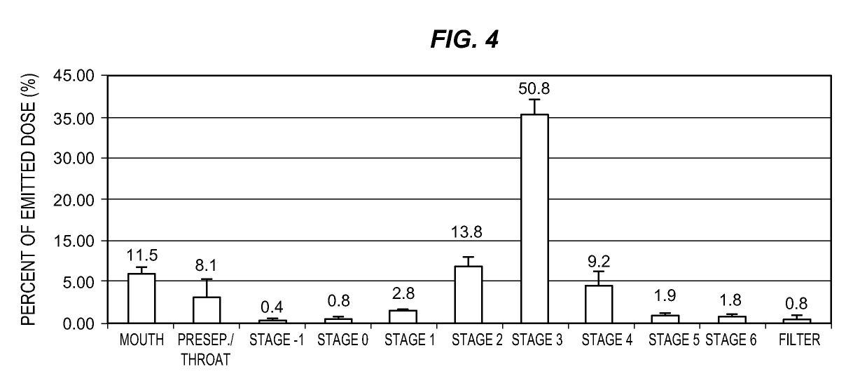

There was excellent reproducibility for aerodynamic properties among the six separate batches of insulin small spherical particles produced as demonstrated by Aerosizer and Andersen Cascade Impactor data. For all six batches, the Aerosizer data indicated that over 99.5% of the particles fell within a size range of 0.63 to 3.4 μm, with a minimum of 60% of the small spherical particles falling within a narrow size range of 1.6 to 2.5 μm (FIG. 3). Statistically, the data indicates that one can be 95% confident that at least 99% of the insulin small spherical particles batches produced have at least 96.52% of the particles in the 0.63 to 3.4 μm size range (−68.5% to 70% of the target diameter of 2 μm).

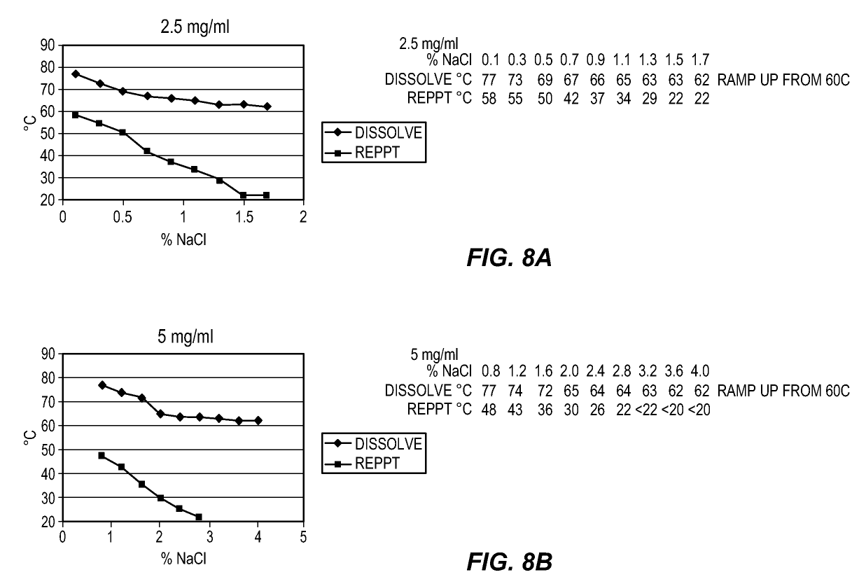

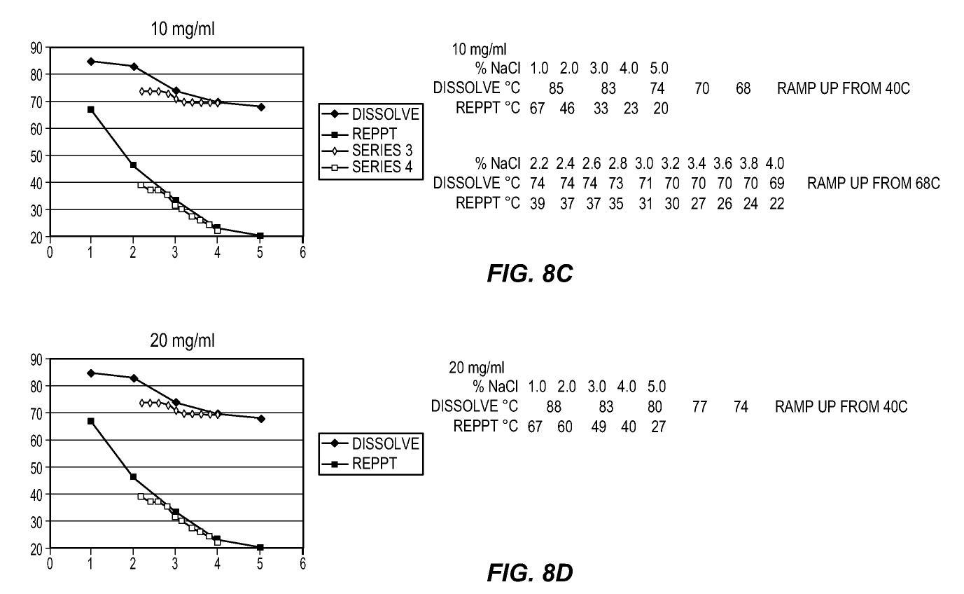

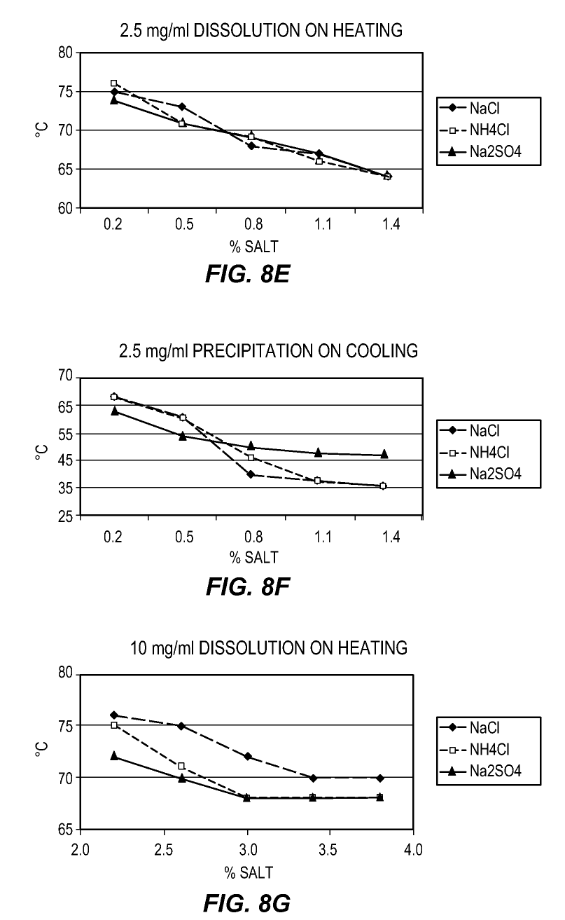

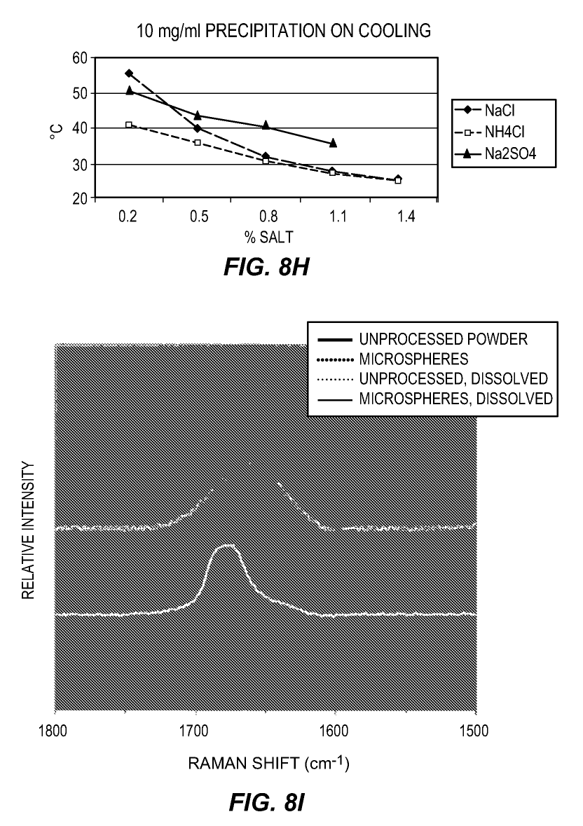

The Andersen Cascade Impactor data corresponded well with the Aerosizer data, with the exception that an average of 17.6% of the dose delivered from the Cyclohaler was deposited in the Mouth and Pre-separator/throat of the apparatus (FIG. 8). The data suggests that the powder dispersion efficiency of the Aerosizer is greater than that of the Cyclohaler device. However, the average emitted dose for the six batches was 71.4% from the Cyclohaler, with 72.8% of the emitted dose deposited on Stage 3 of the impactor. If the respirable fraction for deep lung delivery is estimated to be that fraction with ECD's between 1.1 and 3.3 microns, an average 60.1% of the inhaled insulin small spherical particles may be available for deep lung delivery and subsequent systemic absorption. Excellent reproducibility for the process is shown in Table 1, where the standard deviation values for the MMAD and GSD averages for the six separate batches are extremely low. This indicates that the process variables are under tight control, resulting in batch to batch uniformity for aerodynamic properties.

TABLE 1
Aerodynamic Properties of Insulin Small Spherical Particles

| | MMAD | GSD | % stage 2-F | % stage 3-F | Emitted |
|---|---|---|---|---|---|
| Parameter | (μm) | (μm) | (ECD 3.3 μm) | (ECD 2.0 μm) | dose (%) |
| Mean | 2.48 | 1.51 | 88.8 | 72.8 | 71.4 |
| SD | 0.100 | 0.064 | 4.58 | 4.07 | 5.37 |

Table 1 shows the aerodynamic properties of Insulin small spherical particles. Results (mean +/−SD) were calculated from analysis of separate insulin small spherical particle batches (N=6) on an Andersen Cascade Impactor. Very good reproducibility for the process is demonstrated by the extremely low standard deviations for the MMAD and GSD.

The insulin small spherical particles produced by this cooling process showed little tendency to agglomerate as evidenced by the aerodynamic data in Table 1.

### Example 5

### Stirred Vessel Process for Making Insulin Small Spherical Particles

2880 mL of a buffered polymer solution (18.5% polyethylene glycol 3350, 0.7% sodium chloride, in a 0.1 M sodium acetate buffer, pH 5.65 at 2° C.) was added to a glass 3 liter water jacketed stirred vessel and pre-heated to 75° C. 2.4 grams of human zinc crystalline insulin was suspended in a 80 mL of the buffered polymer solution with sonication to ensure complete dispersion. The insulin suspension was added to the stirred, pre-heated buffered polymer solution, and stirred for an additional 5 minutes. The mixture cleared during this time indicating that the zinc crystalline insulin had dissolved. Water from a chiller set to 10° C. was pumped through the jacket of the vessel until the insulin polymer solution dropped to 15-20° C. The resulting suspension was diafiltered against five volumes of 0.16% sodium acetate-0.026% zinc chloride buffer, pH 7.0, followed by five volumes of deionized water, followed by lyophilization to remove the water. SEM analysis of the lyophilized powder showed uniform small spherical particles with a mean aerodynamic diameter of 1.433 micrometers by TSI Aerosizer time-of flight analysis. Andersen cascade impactor analysis resulted in 73% of the emitted dose deposited on stages 3 to filter, an MMAD of 2.2, and a GSD of 1.6, all indicators of excellent aerodynamic properties of the powder.

### Example 6

### Reduction in the Formation of Insulin Degradation Products by Adjusting the Ionic Strength of a Small Spherical Particle Producing Formulation

Insulin can also be dissolved in the solution at lower initial temperatures, e.g., 75° C., without extended periods of time or an acidic environment, but of which result in significant aggregation, by adding NaCl to the solution.

An improved insulin small spherical particles fabrication process was accomplished using the following technique. A concentrated slurry of zinc crystalline insulin (at room temperature) was added (while stirring) to a 16.7% solution of polyethylene glycol in 0.1 M sodium acetate, pH 5.65, pre-heated to approximately 85 to 90° C. The insulin crystals dissolved completely in this temperature range within five minutes. The insulin small spherical particles formed as the temperature of the solution was lowered.

Significant formation of A₂₁ desamido insulin and insulin dimers due to chemical reactions occurred at initial temperatures of 85-90° C. by the elevated temperatures. However, this required extended periods of time at 75° C. The extended time also resulted in significant insulin degradation. Pre-dissolving the insulin in an acidic environment also caused undesirable conversion of a large percentage of the insulin to an A₂₁ desamido insulin degradation product.

In an experiment, sodium chloride was added to the buffered polymer reaction mixture in an effort to reduce the formation of insulin dimers by chemical means. Although the added sodium chloride did not significantly reduce the formation of desamido or dimer insulin degradation products, the addition of sodium chloride greatly reduced the formation of oligomers (high molecular weight insulin products) (Table 2).

TABLE 2

| | | | | |
|---|---|---|---|---|
| | | | | % other |
| | % | % | % | related |
| Sample Description | dimer | HMWt. | desamido | comps. |

NaCl added to insulin-water suspension

| | | | | |
|---|---|---|---|---|
| control, no added NaCl | 0.94 | 0.23 | 0.78 | 1.52 |
| NaCl, 0.7% final concentration | 0.83 | 0.05 | 0.82 | 1.43 |

NaCl added to polymer solution

| | | | | |
|---|---|---|---|---|
| NaCl, 0.7% final concentration | 0.85 | 0.07 | 0.93 | 1.47 |

In addition, the Zn crystalline insulin dissolved much faster in the presence of NaCl than the control without NaCl. This suggested that addition of sodium chloride improves the rate of solubility of the insulin and allowed a reduction in the temperature used to initially dissolve the zinc insulin crystals. This hypothesis was confirmed in an experiment that demonstrated that the addition of 0.7% NaCl to the formulation allowed the zinc crystalline insulin raw material to dissolve at 75° C. within five minutes, a significantly lower temperature than the 87° C. previously required without NaCl addition. At 75° C., in the absence of NaCl, the insulin did not completely dissolve after 13 minutes.

A series of experiments demonstrated that increasing in the concentration of sodium chloride (2.5 mg/ml, 5.0 mg/ml, 10.0 mg/ml, and 20.0 mg/ml) further reduced the temperature at which the insulin crystals dissolved and also reduced the temperature at which the small spherical particles begin to form (FIGS. 8a-d). Additionally, it was determined that increasing the concentration of the NaCl in the formulation quickly dissolved higher concentrations of Zn crystalline insulin. It was therefore confirmed that the solubility of the insulin at a given temperature could be carefully controlled by adjusting the sodium chloride level of the initial continuous phase. This allows the process to be conducted at temperatures that are less conducive to the formation of degradation products.

In order to determine if the sodium chloride h as unique chemical properties that allow the reduction in temperature to dissolve insulin, equimolar concentrations of ammonium chloride and sodium sulfate, were compared to a control with sodium chloride. Both NH₄Cl and Na₂SO₄ similarly reduced the temperature required to dissolve the zinc crystalline insulin raw material. The higher ionic strength appears to increase the solubility of the insulin in the microsphere producing formulation, without affecting the ability to form small spherical particles as the solution temperature is reduced.

### Example 7

### Study of PEG Concentration on Yield and Insulin Concentration and Size of Insulin Small Spherical Particles

The polyethylene glycol (3350) titration data shows that increasing the PEG-3350 also increases the yield of small spherical particles. However, when the PEG concentration is too high the particles lose their spherical shape, which cancels out the slight improvement in yield.

The insulin concentration data shows a trend opposite to the PEG, where increasing insulin concentration results in a decrease in yield of small spherical particles.

We do see a general trend that higher concentrations of insulin yield larger diameter small spherical particles. In this experiment, the higher concentrations also resulted in a mix of non-spherical particles with the small spherical particles.

### Example 8

### Insulin Small Spherical Particles Study with Dogs

The purpose of this experimental study was to conduct a quantification and visualization experiment for aerosolized insulin powder deposition in the lungs of beagle dogs. ⁹⁹mTc labeled Insulin particles made in accordance with the methods disclosed herein. Pulmonary deposition of the aerosolized insulin was evaluated using gamma scintigraphy.

Five beagle dogs were used in this study and each animal received an administration of an ⁹⁹ mTc radiolabeled insulin particles aerosol. Dog identification numbers were 101, 102, 103, 104, and 105.

Prior to aerosol administration, the animals were anesthetized with propofol through an infusion line for anesthesia and an endotracheal tube was placed in each animal for aerosol delivery.

Each dog was placed in a “Spangler box” chamber for inhalation of the radiolabeled aerosol. Immediately following the radiolabeled aerosol administration, a gamma camera computer image was acquired for the anterior as well as the posterior thoracic region.

Two in-vitro cascade impactor collections were evaluated, one before the first animal (101) aerosol administration and also following the last animal (105) exposure to establish the stability of the ⁹⁹ mTc radiolabeled insulin powder.

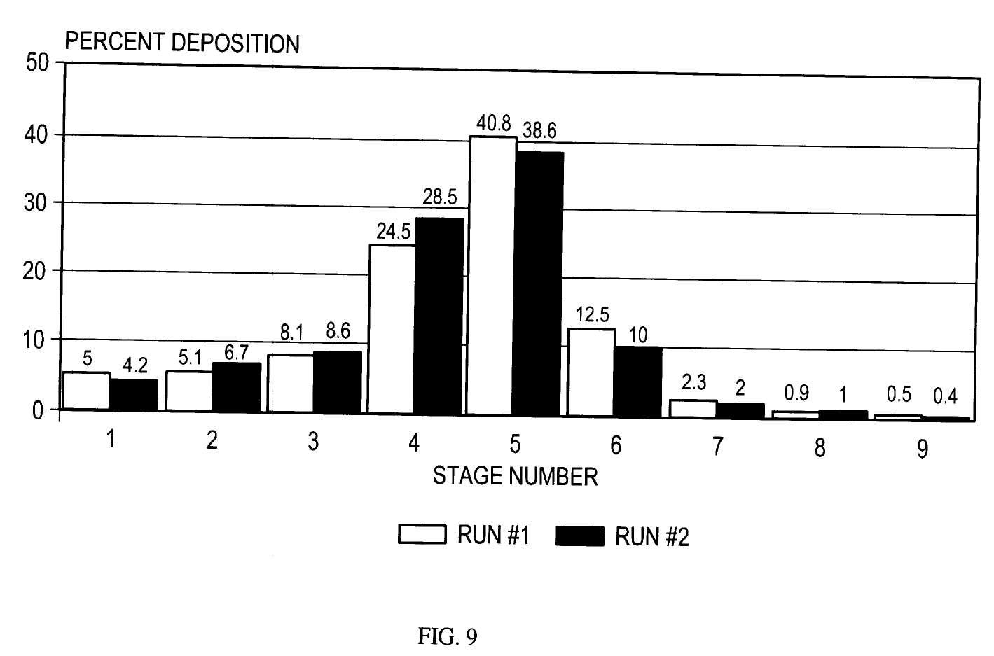

The results are illustrated in FIG. 9. The cascade impactor collections in both cases showed a uni-modal distribution.

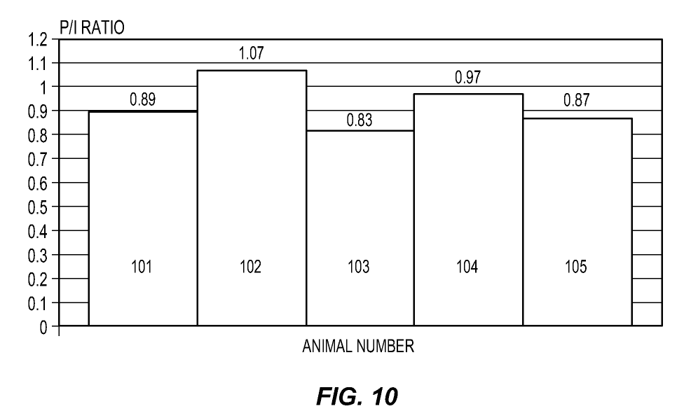

FIG. 10 shows the results for the P/I ratio computations for all animals. The P/I ratio is a measure of the proportion of the ⁹⁹ mTc insulin powder that deposits in the peripheral portions of the lung, i.e., the deep lung. A typical P/I ratio will likely be about 0.7. P/I ratios above 0.7 indicate significant deposition in the peripheral lung compared to central lung or bronchial region.

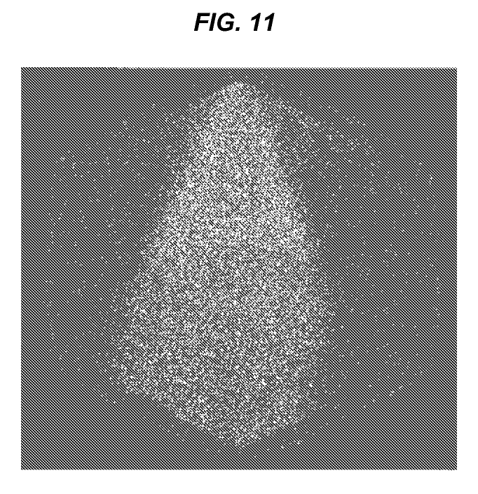

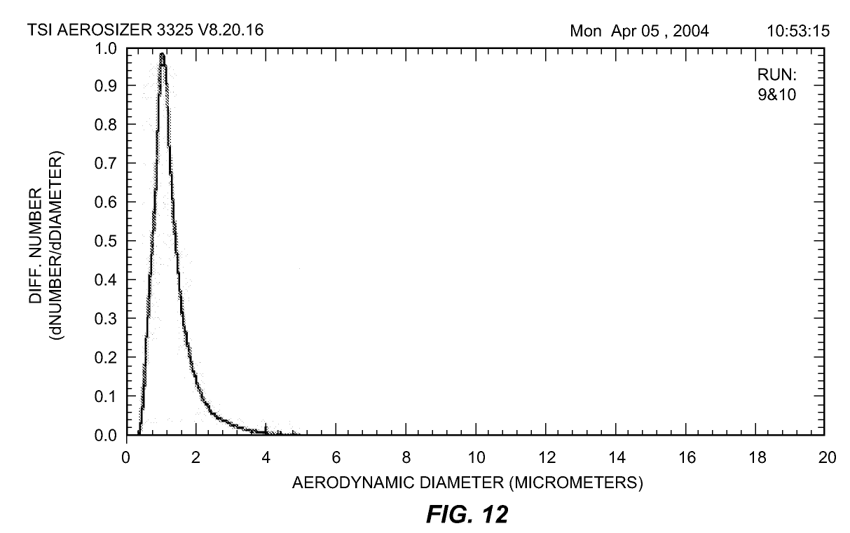

The scintigraphic image in FIG. 11 shows the insulin deposition locations within the respiratory system and is consistent with the P/I data. (FIG. 10) The scintigraphic image for Dog 101 is representative of all 5 dogs in this study.

The scintigraphic image for Dog 101 shows little tracheal or bronchial deposition with an obvious increase in the deposition in peripheral lung. Radioactivity outside the lung is due to rapid absorption of the ⁹⁹ mTc from the deep lung deposition of the aerosolized powder.

The P/I ratios and the image data indicate the ⁹⁹ mTc radiolabeled insulin was deposited primarily in the deep lung. The quantity of the radiolabeled insulin deposited into the peripheral lung was indicative of low levels of agglomeration of the particles.

### Example 9

### Diafiltration Against a Buffer Containing Zinc to Remove Polymer from Insulin Small Spherical Particles

Following fabrication of the insulin small spherical particles in the PSEA solution, it was desirable to remove all of the PSEA from the suspension prior to lyophilization. Even a few percent of residual PSEA could act as a binder to form non-friable agglomerates of the small spherical particles. This agglomeration would adversely affect the emitted dose and aerodynamic properties of powder delivered from DPI devices. In addition, lung tissue exposure to repeated doses of a PSEA could raise toxicology issues.

Three techniques were considered for separation of the small spherical particles from the PSEA prior to lyophilization. Filtration could be used to collect small quantities of particles. However, larger quantities of the small spherical particles quickly blocked the pores of the filtration media, making washing and recovery of more than a few milligrams of particles impractical.

Centrifugation to collect the particles, followed by several wash cycles involving re-suspension in a wash solvent and re-centrifugation, was used successfully to remove the PSEA. Deionized water was used as the wash solvent since the insulin small spherical particles were not readily dissolved and the PSEA remained in solution. One disadvantage of centrifugation was that the small spherical particles were compacted into a pellet by the high g-forces required to spin down the particles. With each successive wash, it became increasingly difficult to resuspend the pellets into discrete particles. Agglomeration of the insulin particles was often an unwanted side effect of the centrifugation process.

Diafiltration using hollow fiber cartridges was used as an alternative to centrifugation for washing the insulin small spherical particles. In a conventional set up of the diafiltration apparatus, the buffered PSEA/insulin particle suspension was placed in a sealed container and the suspension was re-circulated through the fibers with sufficient back-pressure to cause the filtrate to pass across the hollow fiber membrane. The re-circulation rate and back pressure were optimized to prevent blockage (polarization) of the pores of the membrane. The volume of filtrate removed from the suspension was continuously replenished by siphoning wash solvent into the stirred sealed container. During the diafiltration process, the concentration of PSEA in the suspension was gradually reduced, and the insulin small spherical particle suspension was essentially PSEA-free after five to seven times the original volume of the suspension was exchanged with the wash solvent over a period of an hour or so.

Although the diafiltration process was very efficient at removing polymer and very amenable to scaling up to commercial quantities, the insulin small spherical particles did slowly dissolve in the deionized water originally used as the wash solvent. Experiments determined that insulin was gradually lost in the filtrate and the insulin particles would completely dissolve after deionized water equivalent to twenty times the original volume of suspension was exchanged. Although the insulin small spherical particles were found to be sparingly soluble in deionized water, the high efficiency of the diafiltration process continually removed soluble insulin, and probably zinc ions, from the suspension. Therefore, the equilibrium between insoluble and soluble insulin concentration in a given volume of deionized water did not occur with diafiltration, a condition that favored dissolution of the insulin.

Table 3 shows various solutions that were evaluated as potential wash media. Ten milligrams of dry insulin small spherical particles were suspended in 1 mL of each solution and gently mixed for 48 hours at room temperature. The percentage of soluble insulin was measured at 24 and 48 hours. The insulin was found to be sparingly soluble in deionized water, with equilibrium reached at just under 1% of the total weight of insulin soluble in less than 24 hours. However, as previously noted, the high efficiency of diafiltration continuously removes the soluble insulin (and zinc) so this equilibrium is never achieved and the insulin small spherical particles would continue to dissolve. Therefore, insulin solubility in the ideal wash solution would be below that of water. Since insulin is least soluble near its isoelectric point, acetate buffers at two molarities and pH 5.65 were examined. The solubility of the insulin was found to be dependent on the molarity of the buffer, and comparable to water at low molarities. Ethanol greatly reduced the solubility of the insulin but only at near anhydrous concentrations. The insulin solubility would actually increase when ethanol mixed with water solutions were used in the PSEA/insulin small spherical particle suspension in the early stages of diafiltration.

TABLE 3
Insulin small spherical particle solubility in various wash solutions

| | % dissolved | % dissolved |
|---|---|---|
| | insulin | insulin |
| Wash Solution | after 24 hours | after 48 hours |
| Deionized water | 0.91 | 0.80 |
| 0.1 M sodium acetate, pH 5.65 | 2.48 | 2.92 |
| 0.001 M sodium acetate, pH 5.65 | 0.54 | 0.80 |
| 0.16% sodium acetate-0.016% | 0.14 | 0.11 |
| ZnO, Ph 5.3 | | |
| 0.16% sodium acetate-0.027% | 0.09 | 0.06 |
| ZnCl₂, pH 7.0 | | |
| 50% ethanol/deionized | 9.47 | 9.86 |
| water (v/v) | | |
| 100% anhydrous ethanol | 0.05 | 0.04 |

Buffer solutions used in commercial zinc crystalline insulin suspensions for injection also contain zinc in solution. Two of these solutions were tested with insulin small spherical particles and found to greatly reduce insulin solubility compared to deionized water. According to the literature, zinc crystalline insulin should have 2 to 4 Zn ions bound to each insulin hexamer. Zinc ions per hexamer ranged from 1.93 to 2.46 for various zinc crystalline insulin preparations used as the raw material for making the insulin small spherical particles. This corresponded to 0.36 to 0.46% zinc per given weight of raw material zinc crystalline insulin. After formation of the insulin small spherical particles and diafiltration against deionized water, 58 to 74% of the zinc was lost during processing. The loss of zinc from the insulin particles would cause increased solubility of the insulin and loss during diafiltration.

Diafiltering the insulin small spherical particles against 0.16% sodium acetate-0.027% ZnCl₂, pH 7.0, virtually eliminated insulin loss in the filtrate. Surprisingly however, the zinc content of the insulin small spherical particles increased to nearly 2%, well above the 0.46% measured for the starting zinc crystalline insulin raw material. Another unexpected result of diafiltration against zinc containing buffer was a dramatic improvement in the emitted dose observed from a Cyclohaler DPI device (68% diafiltered against deionized water versus 84 to 90% after zinc buffer diafiltration) and a decrease in the amount of insulin particles deposited in the throat of the Andersen Cascade Imp actor. The zinc buffer diafiltration improved the dispersability of the insulin small spherical particle dry powder and reduced agglomeration of the particles, resulting in lower MMAD's and higher deposition on lower stages of the impactor. This suggested that the zinc buffer diafiltration and higher zinc content in the insulin small spherical particles could improve the percent of the dose deposited in the deep lung.

When suspended in the propellant HFA-134a without added excipients for use in an MDI application, there was no apparent irreversible agglomeration of the zinc buffer washed insulin small spherical particles. The insulin particles did flocculate out of suspension in less than a minute, but readily resuspended when shaken just before use. Shaking the MDI container just before use is normally part of the instructions given for using any MDI product. In fact, the loose flocculated particles that settle on the bottom of the MDI container may actually inhibit long term agglomeration of the insulin particles (in addition to the minimal contact due to their spherical shape) since the particles do not settle into a densely packed layer on the bottom of the MDI pressurized container. Therefore, properties imparted by the zinc buffer diafiltration of the insulin small particles may improve the long term shelf life and dispersability of MDI preparations for insulin and other zinc binding compounds.

Since the insulin small spherical particles were found to be noncrystalline by XRPD analysis, the zinc binding was not associated with zinc ion coordination of insulin monomers to form hexamers. Therefore, the non-specific binding of ions and resulting potential benefits could extend to the binding of ions other than zinc. Different proteins that do not bind zinc could bind other ions that would reduce solubility in the diafiltration process and impart similar beneficial effects.

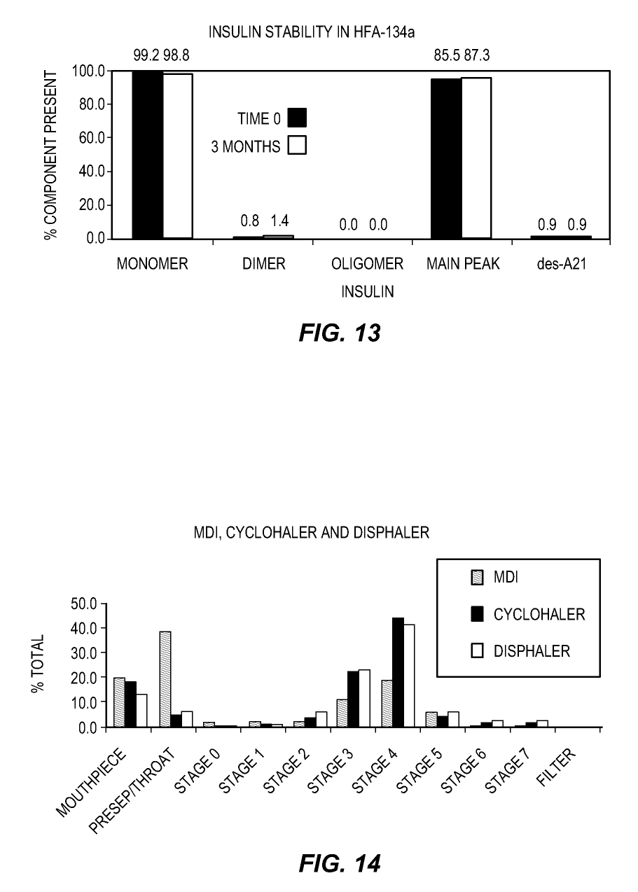

The small spherical particles were suspended in Hydro Fluoro Alkane (HFA) 134a propellant at a concentration of 10 mg/mL. The chemical stability of the insulin after storage in the HFA 134a was assessed at time 0 and at one month. The data shown in FIG. 13 shows the preservation of the insulin microspheres in terms of monomeric insulin, insulin dimer, insulin oligomers, insulin main peak and A21-desamindo insulin.

In the following study, insulin small spherical particles prepared according to the methods in Example 4 were compared as to their performance in three different inhalation devices using the Andersen Cascade Impactor method. The Cyclohaler device is a commercial dry powder inhaler (DPI), the Disphaler is another dry powder inhaler and the metered dose inhaler (MDI) is a device in which the microspheres are suspended in HFA 134a as described in this example and are propelled through a 100 microliter or other sized metering valve. The results in FIG. 14 clearly show that the small spherical particles impacting the stages of the Andersen Cascade Impactor device deposit on stages 3 and 4. This is indicative of a very reproducible performance of the small spherical particles regardless of the device used as an inhaler. The only major difference between the DPI and MDI devices is the significantly greater quantity of small spherical particles deposited in the throat section of Andersen Cascade Impactor using the MDI. The high velocity that the MDI device propels the small spherical particles against the throat of the Andersen Impactor explains the higher proportion of insulin microspheres deposited compared to the DPI devices. It can be assumed by those skilled in the art that an MDI device with an attenuated or modified exit velocity could be used to decrease the number of the small spherical particles depositing in the throat. Additional measures could be the use of spacer devices at the end of the MDI.

Insulin small spherical particles (Lot number YQ010302) were fabricated from lyophilized insulin starting material according to the methods described in this example. One year storage stability for the insulin small spherical particles was compared with the lyophilized insulin starting material at 25° C. and 37° C. The insulin stability was compared by examining Total Related Insulin Compounds, Insulin Dimers and Oligomers and A21-desamido Insulin.

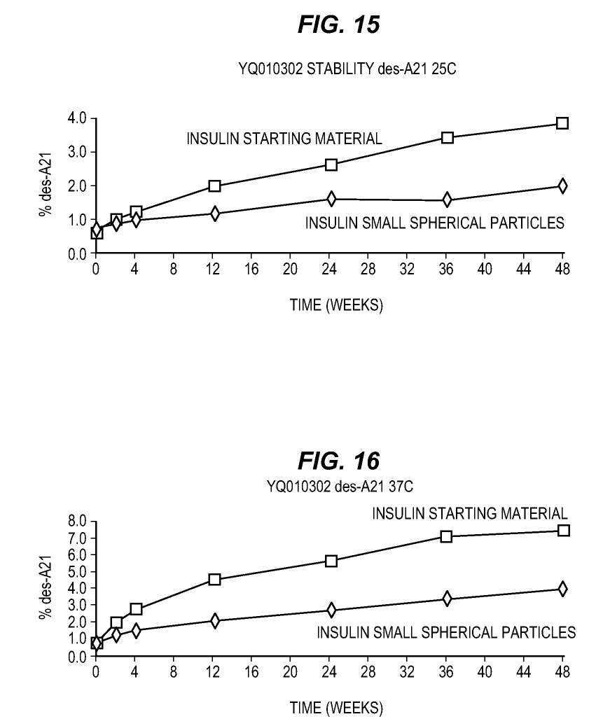

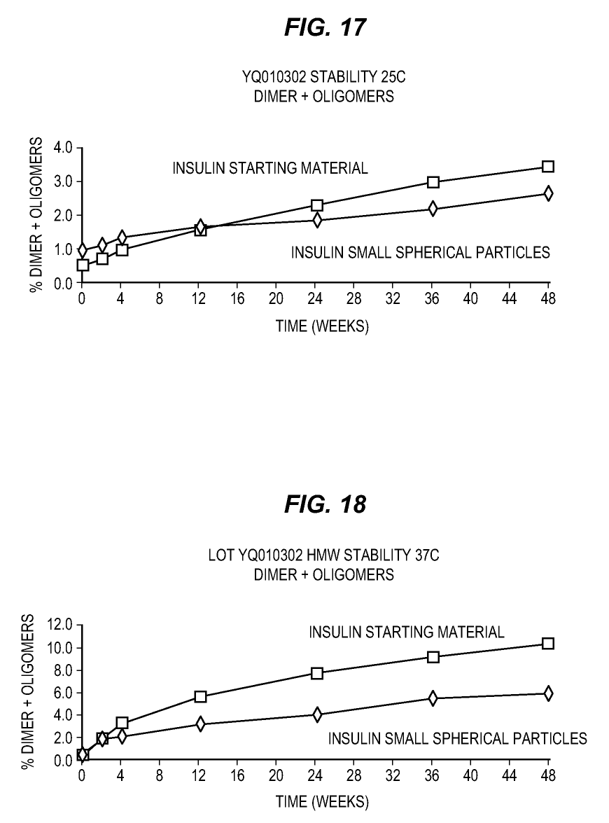

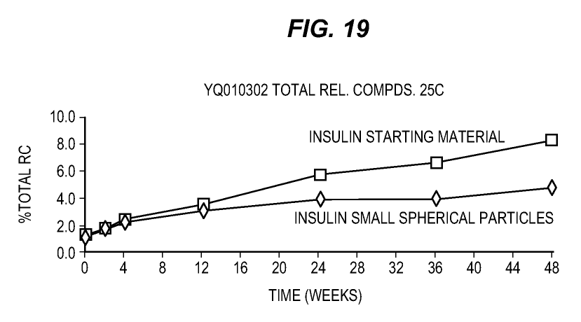

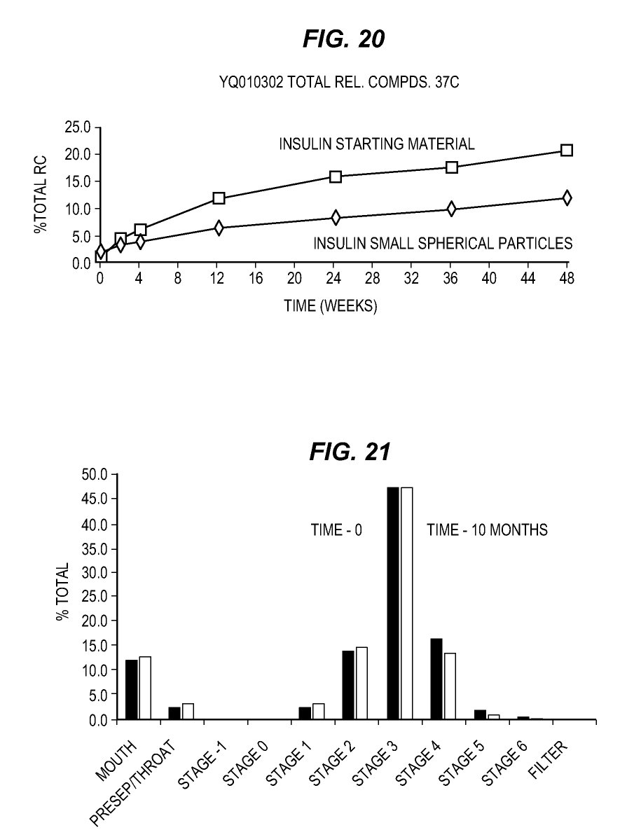

FIGS. 15-20 show that over a one year period, the insulin small spherical particles exhibited significantly lower amounts of Insulin Dimers and Oligomers, A21-desamido Insulin and Total Related Insulin Compounds and compared to insulin starting material stored under the same conditions. This indicates that the microsphere form of insulin is significantly more stable to chemical changes than the starting material.

Insulin small spherical particles were tested in the Andersen Cascade Impactor study at 0 time and 10 months after manufacture. A Cyclohaler DPI device was used to determine the aerodynamic stability after long term storage. FIG. 21 shows that the aerodynamic performance remains remarkably consistent after 10 months storage.

Raman spectroscopic investigation was undertaken to elucidate structural differences between unprocessed insulin sample and the insulin in the small spherical particles prepared in this Example. It was shown that the insulin in the small spherical particles possess substantially higher .beta.-sheet content and subsequently lower .alpha.-helix content than their parent unprocessed insulin sample. These findings are consistent with the formation of aggregated microfibril structures in small spherical particles. However, when dissolved in an aqueous medium, the spectra reveal essentially identical protein structures resulting from either unprocessed microspheres or insulin, indicating that any structural changes in microspheres are fully reversible upon dissolution.

Two batches of insulin were tested using Raman spectroscopy: A) unprocessed Insulin USP (Intergen, Cat N.4502-10, Lot# XDH 1350110) and B) Insulin in the small spherical particles (JKPL072502-2 NB 32: P.64). The powderous samples or insulin solutions (about 15 mg/mL in 0.01 M HCl) were packed into standard glass capillaries and thermostated at 12° C. for Raman analysis. Typically, a 2-15 μL aliquot was sufficient to fill the portion of the sample capillary exposed to laser illumination. Spectra were excited at 514.5 nm with an argon laser (Coherent Innova 70-4 Argon Ion Laser, Coherent Inc., Santa Clara, Calif.) and recorded on a scanning double spectrometer (Ramalog V/VI, Spex Industries, Edison, N.J.) with photon-counting detector (Model R928P, Hamamatsu, Middlesex, N.J.). Data at 1.0 cm⁻¹ intervals were collected with an integration time of 1.5 s and a spectral slit width of 8 cm⁻¹. Samples were scanned repetitively, and individual scans were displayed and examined prior to averaging. Typically, at least 4 scans of each sample were collected. The spectrometer was calibrated with indene and carbon tetrachloride. Spectra were compared by digital difference methods using SpectraCalc and GRAMS/AI Version 7 software (Thermo Galactic, Salem, N.H.). The spectra were corrected for contributions of solvent (if any) and background. The solutions' spectra were corrected by acquiring 0.01M HCl spectrum under identical conditions and fit with a series of five overlapping Gaussian-Lorentzian functions situated on a sloping background [S.-D. Yeo, P. G. Debenedetti, S. Y. Patro, T. M. Przybycien, J. Pharm. Sci., 1994, 83, 1651-[656]. The fitting was performed in the 1500-1800 cm⁻¹ region.

Raman spectra were obtained for both powderous insulin samples and their respective solutions (FIG. 8i). The spectrum of the un-processed sample corresponds to the previously described spectra of the commercial insulin samples very well [S.-D. Yeo, P. G. Debenedetti, S. Y. Patro, T. M. Przybycien, J. Pharm. Sci., 1994, 83, 1651-1656; J. L. Lippert, D. Tyminski, P. J. Desmueles, J. Amer. Chem. Soc., 1976, 98, 7075-7080]. The small spherical particle sample exhibited a pronounced (about +10 to +15 cm⁻¹) shift in the amide I mode, indicative of a significant perturbation in the secondary structure of the protein. Notably, however, spectra of the commercial powder and small spherical particles were virtually identical when the samples were dissolved in the aqueous medium, indicating that the changes in the secondary structure upon processing were completely reversible.

The secondary structural parameters were estimated using the computing algorithm that included smoothing, subtraction of the fluorescence and aromatic background, and the amide I bands deconvolution. The exponentially decaying fluorescence was subtracted essentially as described elsewhere [S.-D. Yeo, P. G. Debenedetti, S. Y. Patro, T. M. Przybycien, J. Pharm. Sci., 1994, 83, 1651-1656]. The estimated structural parameters are collected in Table 4.

TABLE 4
Structural parameters of insulin samples
estimated from Raman spectra.

| | Total α-helix | Total β-sheet | β-Reverse | Random |
|---|---|---|---|---|
| Sample | content, % | content, % | turn, % | coil, % |
| Unprocessed, | 44 | 31 | 14 | 11 |
| Powder | | | | |
| Unprocessed insulin | 44 | 28 | 11 | 17 |
| in solution | | | | |
| small spherical | 11 | 67 | 15 | 7 |
| particles, powder | | | | |
| small spherical | 44 | 30 | 11 | 15 |
| particles in solution | | | | |

### Example 10

### Preparation of Small Spherical Particles of Human Insulin by an Isothermal Method

Human insulin USP (Intergen) was dispersed in a NaCl and PEG (MW 3350, Spectrum Lot# RP0741) solution resulting in final insulin concentration of 0.86 mg/mL, and 0.7 wt % NaCl and 8.3 wt % PEG concentrations. The pH was adjusted to 5.65 by addition of minute amounts of glacial acetic acid and 1 M NaOH solutions. After heating to T₁=77° C., clear protein solutions were obtained resulting in the insulin concentration C_{eq}. Then the solutions were cooled at a predetermined rate to a temperature T₂=37° C. At the T₂, protein precipitation was observed. The precipitates were removed by centrifugation (13,000×g, 3 min), again at temperature 37° C., and the insulin concentration (C*) in the resulting supernatant was determined by bicinchoninic protein assay to be 0.45 mg/mL. Thus prepared insulin solution that is kept at 37° C. is designated Solution A.

Solution B was prepared by dissolution of human insulin in 0.7 wt % NaCl/8.3 wt % PEG (pH brought to about 2.1 by HCl addition) resulting in 2 mg/mL insulin concentration. The solution was incubated at 37° C. with stirring for 7 h and subsequently sonicated for 2 min. Aliquots of the resulting Solution B were added to Solution A resulting in total insulin concentration of 1 mg/mL. The resulting mixture was kept under vigorous stirring at 37° C. overnight resulting in insulin precipitates, which were gently removed from the liquid by using a membrane filter (effective pore diameter, 0.22 μm). The resulting protein microparticles were then snap-frozen in liquid nitrogen and lyophilized.

Microencapsulation of Pre-Fabricated Small Spherical Particles

### Example 11

### Preparation of PLGA-Encapsulated Pre-Fabricated Insulin Small Spherical Particles

a) A 20% (w/v) polymer solution (8 ml) was prepared by dissolving 1600 mg of a Polylactide-co-glycolide (PLGA, MW 35 k) in methylene chloride. To this solution was added 100 mg of insulin small spherical particles (INSms), and a homogenous suspension was obtained my vigorous mixing of the medium using a rotor/stator homogenizer at 11 k rpm. The continuous phase consisted of 0.02% aqueous solution of methylcellulose (24 ml) saturated with methylene chloride. The continuous phase was mixed at 11 k rpm using the same homogenizer, and the described suspension was gradually injected to the medium to generate the embryonic microencapsulated particles of the organic phase. This emulsion has an O/W ratio of 1:3. The emulsification was continued for 5 minutes. Next, the emulsion was immediately transferred into the hardening medium consisted of 150 ml deionized (DI) water, while the medium was stirred at 400 rpm. The organic solvent was extracted over one hour under reduced pressure at −0.7 bar. The hardened microencapsulated particles were collected by filtration and washed with water. The washed microencapsulated particles were lyophilized to remove the excess water. The resultant microencapsulated particles had an average particle size of about 30 μm with majority of the particle population being less than 90 μm, and contained 5.7% (w/w) insulin.

b) A 30% (w/v) polymer solution (4 ml) was prepared by dissolving 1200 mg of a 50:50 polylactide-co-glycolide (PLGA, MW 35 k) in methylene chloride. Next a suspension of 100 mg INSms in the described polymer solution was prepared using a homogenizer. This suspension was used to generate the O/W emulsion in 12 ml 0.02% aqueous solution of methylcellulose as described in Example 11 (W/O ratio=1:3). The same procedures as Example 11 are followed to prepare the final microencapsulated particles. The microencapsulated particles formed had an average particle size of 25 μm, ranging from 0.8 to 60 μm. The insulin content of these microencapsulated particles was 8.8% (w/w).

Alternatively, a 10% (w/v) solution of the polymer was used to perform the microencapsulation process under the same conditions described. This process resulted in microencapsulated particles with an average particle size of about 12 μm with most the particles less than 50 μm, and an insulin loading of 21.1% (w/w).

Method For In Vitro Release: The in vitro release (IVR) of insulin from the microencapsulated particles is achieved by addition of 10 ml of the release buffer (10 mM Tris, 0.05% Brij 35, 0.9% NaCl, pH 7.4) into glass vials containing 3 mg equivalence of encapsulated insulin, incubated at 37° C. At designated time intervals 400 μL of the IVR medium is transferred into a microfuge tube and centrifuged for 2 min at 13 k rpm. The top 300 μL of the supernatant is removed and stored at −80° C. until analyzed. The taken volume was replaced with 300 μL of the fresh medium, which was used to reconstitute the pallet along with the remaining supernatant (100 μL). The suspension is transferred back to the corresponding in vitro release medium.

### Example 12

### Procedure for Microencapsulation of Pre-Fabricated Insulin Small Spherical Particles in PLGA/PLA Alloy Matrix System

A 30% (w/v) solution of a PLGA/PLA alloy was prepared in methylene chloride (4 ml). The alloy consisted of a 50:50 PLGA (MW 35 k), D,L-polylactic acid (PLA, MW 19 k) and poly L-PLA (PLLA, MW 180 k) at 40, 54 and 6% (0.48, 0.68 and 0.07 g), respectively. The same procedures as Example 11b were followed to prepare the final microencapsulated particles. The examples of the microencapsulated particles had a particle size range of 0.8-120 μm, averaging at 40 μm with most of the particles population smaller than 90 μm.

### Example 13

### Procedure for Microencapsulation of Pre-Fabricated Insulin Small Spherical Particles in PLGA Matrix System Using PEG in Both Continuous and Discontinuous Phases

A solution of 4 ml of 10% 50:50 PLGA (0.4 g) and 25% polyethylene glycol (PEG, MW 8 k) was prepared in methylene chloride. Using a rotor/stator homogenizer, 100 mg of the INSms were suspended in this solution at 11 k rpm. The continuous phase consisted of aqueous solution (12 ml) of 0.02% (w/v) methylcellulose and 25% PEG (MW 8 k) saturated with methylene chloride. The continuous phase was mixed at 11 k rpm using the same homogenizer, and the described suspension was gradually injected to the medium to generate the embryonic microencapsulated particles of the organic phase. This emulsion has an O/W ratio of 1:3. The emulsification was continued for 5 minutes. Then, the emulsion was immediately transferred into the hardening medium consisted of 150 ml DI-water, while the medium was stirred at 400 rpm. The organic solvent was extracted over one hour under reduced pressure at −0.7 bar. The hardened microencapsulated particles were collected by filtration and washed with water. The washed microencapsulated particles were lyophilized to remove the excess water. The microencapsulated particles of this example had an average particle size of 30 μm, ranging from 2 to 90 μm with majority of the population being smaller than 70 μm. The insulin content of these microspheres was 16.0% (w/w).

### Example 14

### Procedure for Microencapsulation of Pre-Fabricated Insulin Small Spherical Particles in PLGA Matrix System at Various Ph of Continuous Phase Using Phosphate Buffer

A solution of 4 ml of 20% 50:50 35 kD PLGA (0.8 g) was prepared in methylene chloride. Using a rotor/stator homogenizer, 100 mg of the INSms were suspended in this solution at 11 k rpm. The continuous phase consisted of aqueous solution of 0.1% (w/v) methylcellulose and 50 mM phosphate buffer at pH 2.5, 5.4 and 7.8. Microencapsulation was performed using the continuous setup (FIG. 22A). The continuous phase was mixed at 11 k rpm and fed into the emulsification chamber at 12 m/min. The dispersed phase was injected into the chamber at 2.7 ml/min to generate the embryonic microencapsulated particles. The produced emulsion was removed from the chamber and transferred into the hardening bath in a continuous fashion. The hardening medium was stirred at 400 rpm. The organic solvent was extracted over one hour under reduced pressure at −0.4 bar. The hardened microencapsulated particles were collected by filtration and washed with water. The washed microencapsulated particles were lyophilized to remove the excess water.

The insulin contents of the resultant microencapsulated particles s prepared at pH 2.5, 5.4 and 7.8 were estimated to be 12.5, 11.5 and 10.9, respectively. The results of size distribution analysis of the microencapsulated particles are summarized in Table 5.

TABLE 5
Size distribution of insulin loaded- PLGA microencapsulated
particles fabricated at various pH of the continuous phase.

| | Particle size (μm) | | | |
|---|---|---|---|---|
| pH of Continuous Phase | Range | Average | 95% Under | 5% Under |
| 2.5 | 1.4-54 | 24 | 35.9 | 13.8 |
| 5.4 | 0.9-46 | 23 | 33.8 | 11.8 |
| 7.8 | 0.8-25 | 11 | 16.0 | 5.7 |

Method For in vitro Release: The in vitro release of insulin from the microencapsulated particles was achieved by addition of 10 ml of the release buffer (10 mM Tris, 0.05% Brij 35, 0.9% NaCl, pH 7.4) into glass vials containing 3 mg equivalence of encapsulated insulin, incubated at 37° C. At designated time intervals 400 μL of the IVR medium was transferred into a microfuge tube and centrifuged for 2 min at 13 k rpm. The top 300 μL of the supernatant was removed and stored at −80° C. until analyzed. The taken volume was replaced with 300 μL of the fresh medium, which was used to reconstitute the pallet along with the remaining supernatant (100 μL). The suspension was transferred back to the corresponding in vitro release medium.

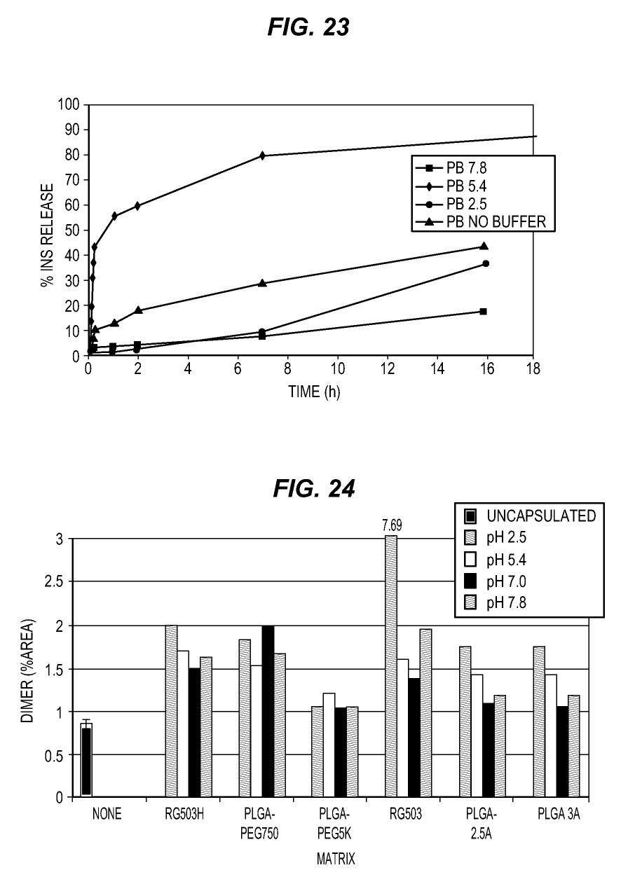

The in vitro release (IVR) results of the above preparations are shown in FIG. 23, and indicate the significant effect of pH of the continuous phase on release kinetics of insulin from the formulations.

### Example 15

### Determination of Integrity of Microencapsulated Pre-Fabricated Insulin Small Spherical Particles

To assess the effect of the microencapsulation process on integrity of encapsulated pre-fabricated insulin small spherical particles, the polymeric microencapsulated particles containing the pre-fabricated INSms were deformulated using a biphasic double extraction method. A weighed sample of the encapsulated INSms were suspended in methylene chloride and gently mixed to dissolve the polymeric matrix. To extract the protein, a 0.01 N HCl was added and the two phases were mixed to create an emulsion. Then, the two phases were separated, the aqueous phase was removed and refreshed with the same solution and the extraction process was repeated. The integrity of the extracted insulin was determined by size exclusion chromatography (SEC). This method identifies extend of monomer, dimer and high molecular weight (HMW) species of INS in the extracted medium. Appropriate controls were used to identify the effect of the deformulation process on the integrity of INS. The results showed no significant effect of this process on INS integrity.

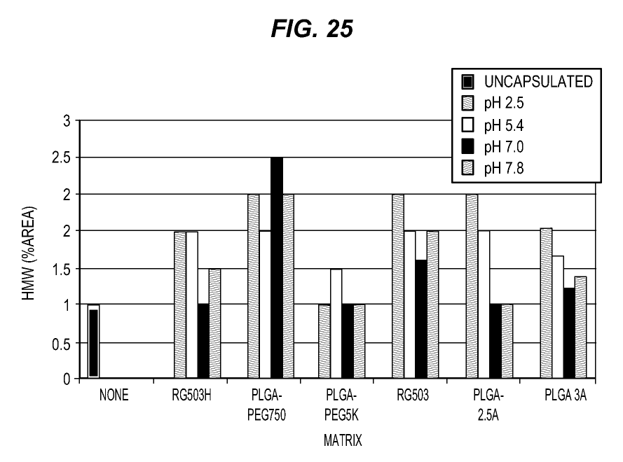

The encapsulated INSms contained 97.5-98.94% monomers of the protein, depending on the conditions and contents of the microencapsulation process, in comparison with 99.13% monomer content in the original INSms (unencapsulated). Content of the dimer species in the encapsulated INSms ranged from 1.04% to 1.99% in comparison with 0.85% in the original INSms. The HMW content of the encapsulated INSms ranged from 0.02% to 0.06% versus 0.02% in the original INSms. The results are summarized in Table 6. The effect of polymeric matrix is depicted in FIGS. 24 and 25.

TABLE 6
Effect of the microencapsulation process on integrity of encapsulated
pre-fabricated insulin small spherical particles.

| | Monomer (%) | Dimer (%) | HMW (%) |
|---|---|---|---|
| Unencapsulated INSms | 99.13 | 0.85 | 0.02 |
| Encapsulated INSms | 97.5-98.94 | 1.04-1.99 | 0.02-0.06 |

### Example 16

### In Vivo Release Insulin from Microencapsulated Pre-Fabricated Insulin Small Spherical Particles

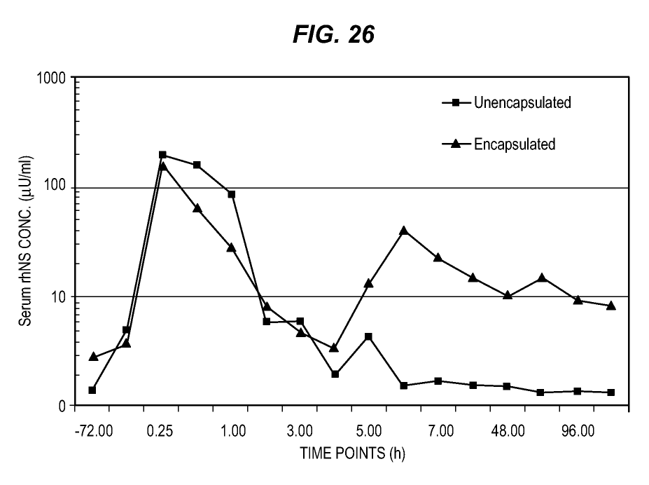

In vivo release of insulin from the microencapsulated particles of pre-fabricated insulin small spherical particles was investigated in Sprague Dawley (SD) rats. The animals received an initial subcutaneous dose of 1 IU/kg of the unencapsulated or encapsulated pre-fabricated insulin small spherical particles. ELISA was used to determine the recombinant human insulin (rhINS) serum levels in the collected samples. The results are illustrated in FIG. 26.

### Example 17

### Method for Production of Insulin Pulmonary Microspheres in 1.5 mL Microcentrifuge Tube

One ml of 10 mg/ml insulin solution was prepared (this solution is prepared just prior to use). Per milliliter of solution, 10 mg of insulin (Zn) was mixed in 0.99 mL of degassed deionized water. The suspension would be cloudy. 10 microliters of 1 N HCl per mL of solution was added and mixed. The solution should clear with mixing. If the solution did not clear, smaller volumes of 1N HCl were added until the insulin was in solution. 0.8 ml of PEG/PVP (12.5%/12.5% PVP in 0.1 M Sodium acetate, pH 5.65) in 0.1 M Sodium acetate, pH 5.65 was added to 0.40 mL of insulin solution and mixed gently in a 1.5 mL polypropylene microcentrifuge tube. The solution turned cloudy.

The microcentrifuge tube was placed into the 90° C. water bath for 30 minutes. The microcentrifuge tube was removed from the water bath and cooled on bench at room temperature for 30 minutes. The microcentrifuge tube was centrifuged in a microcentrifuge for 10 minutes at 8000 RPM. The supernatant was decanted. Deionized water was added and the pellet was resuspended. The microcentrifuge tube was centrifuged in a microcentrifuge for 10 minutes at 6000 RPM. The supernatant was decanted. Deionized water was added, the pellet was resuspended and repeated. The microsphere pellet was resuspended in 5 mL of deionized water and the pellet was lyophilized. The resulting lyophilized spheres yielded 1 micron sized Insulin spheres by laser light scattering which assayed to be >95% wt/wt insulin.

### Example 18

### Method for Fabricating Insulin Pulmonary Microspheres Via Continuous Flow Through

Ten ml of 10 mg/ml insulin solution was prepared (this solution was prepared just prior to use). Per milliliter of solution, 100 mg of insulin (Zn) was mixed in 9.8 mL of degassed deionized water. The suspension would be cloudy. 100 microliters of 1 N HCl was added per mL of solution and mixed. The solution would clear with mixing. If the solution did not clear, smaller volumes of 1N HCl were added until the insulin was in solution. 20 ml of PEG/PVP (12.5%/12.5%) in 0.1 M Sodium acetate, pH 5.65 was added to 10 mL of insulin solution and mixed gently. The solution would turn cloudy.

Fabrication apparatus set-up: Eight feet of ⅛ inch o.d. ( 3/32 i.d.) polypropylene tubing was prepared. It was ensured that 4 feet were submerged in the water bath in a loop of about 6 inches diameter, with the inlet connected to the Rainin peristaltic pump and the outlet to an empty collection vessel. A three (3) foot cooling loop was allowed between the water bath and the collection vessel. The water bath was heated to 90° C. Note: It is important to not allow any air to enter tubing after beginning to pump solutions through the tubing. Air bubbles can cause aggregation and clogging.

Microsphere production procedure: The Rainin peristaltic pump speed was set to a setting that is approximately a 1 mL/minute flow rate. Immediately prior to starting the run, about 10 mL of the diluted, degassed polymer solution (1 part deionized water to 2 parts PEG/PVP (12.5%/12.5%) was pumped in 0.1 M sodium acetate, pH 5.65) through the tubing to equilibrate the temperature of the cooling zone. The pump was stopped momentarily to avoid drawing a bubble into the tubing. The inlet side of the tubing was carefully transferred to the insulin/polymer raw material suspension. The collection vessel was switched to an empty container before the Insulin microspheres exited the tubing. The first Insulin microspheres exited the tubing much faster than expected from the actual flow rate of the pump due to laminar flow. A thin line of Insulin raw material suspension was observed running through the pre-filled polymer solution in the tube, that gradually widened to the inside diameter of the tubing. A small air bubble was allowed, then the polymer/buffer solution which has been diluted by one third (1:3) with deionized water to pump through tubing following the insulin-PEG/PVP solution. The collection vessel was removed for further processing after the microsphere collection was completed.

Microsphere washing procedure: The microsphere suspension was diluted with approximately an equal volume of deionized water in order to reduce the viscosity of the suspension. Using a 50 mL polypropylene centrifuge tube, the microsphere suspension was spun at 3500×g for 15 minutes. The supernatant was carefully decanted from the pellet. The pellet in each tube was resuspended with deionized water equivalent to the original volume in the tube and then vortexed until all of the pellet has been completely resuspended. The suspension was centrifuged at 3000×g for 15 minutes. The supernatant was carefully decanted from the pellet. The pellet in each tube was resuspended with deionized water equivalent to the original volume in the tube and then vortexed until all of the pellet has been completely resuspended. The suspension was centrifuged at 3000×g for 15 minutes. The supernatant was carefully decanted from the pellet. The pellet in each tube was resuspended with deionized water equivalent to ⅔ the volume of the tube and then vortexed until all of the pellet has been completely resuspended. The suspension was homogenized with a homogenizer (e.g., IKA Homogenizer at speed setting 3 for 2 minutes) and centrifuged at 3000×g for 15 minutes. The supernatant was carefully decanted from the pellet. The pellet in each tube was resuspended with a minimum volume of deionized water and then vortexed until all of the pellet has been completely resuspended. The suspension was homogenized with the IKA Homogenizer at speed setting 3 for 2 minutes or equivalent. The microspheres were transferred to an appropriate sterile vessel and diafiltration was performed to wash microspheres until all free polymer has been removed. The microsphere suspension was concentrated using the hollow fiber cartridge system prior to lyophilization. The microspheres were bulk lyophilized under sanitary conditions, and stored dry until ready for filling.

### Example 19

### Method for Production of Insulin Pulmonary Microspheres in 2 Foot Length (60 Ml) Glass Chromatography Column (General Batch-Wise Process)

The water jacketed chromatography column was preheated to 90° C. A 10 mg/mL insulin solution was prepared in degassed, deionized water as described in Example 1. A 12.5% PEG (3350), 12.5% PVP (K12) in 0.1 M sodium acetate buffer was prepared. 20 mL of the insulin solution was mixed with 40 mL of the polymer solution (the final insulin concentration was 3.33 mg/mL). These suspensions were initially at room temperature of about 25° C. The insulin/polymer suspension was pumped into the preheated chromatography column. Then incubated for 30 minutes at 90° C. The temperature was ramped down to 25° C. over 1.5 hours. The suspension was pumped from the column out into a collection vessel. Deionized water was added.

Microsphere Washing Procedure: The microsphere suspension was diluted with approximately an equal volume of deionized water in order to reduce the viscosity of the suspension. Using 50 mL polypropylene centrifuge tubes, the microsphere suspension was spun at 3500×g for 15 minutes. The supernatant was carefully decanted from the pellet. The pellet in each tube was resuspended with deionized water equivalent to the original volume in the tube and vortexed until all of the pellet has been completely resuspended. The suspension was centrifuged at 3000×g for 15 minutes. The supernatant was carefully decanted from the pellet. The pellet in each tube was resuspended with deionized water equivalent to the original volume in the tub and vortexed until all of the pellet was completely resuspended. The suspension was centrifuged at 3000×g for 15 minutes. The supernatant was carefully decanted from the pellet. The pellet in each tube was resuspended with deionized water equivalent to ⅔ the volume of the tube and then vortexed until all of the pellet has been completely resuspended. The suspension was homogenized with the IKA Homogenizer at speed setting 6 for 2 minutes then centrifuged at 3000×g for 15 minutes. The supernatant was carefully decanted from the pellet. The pellet in each tube was resuspended with a minimum volume of deionized water and vortexed until all of the pellet was completely resuspended. The suspension was homogenized with the IKA Homogenizer at speed setting 6 for 2 minutes. The microspheres were transferred to an appropriate sterile vessel and diafiltration was performed to wash microspheres until all free polymer has been removed. The microsphere suspension was concentrated using the hollow fiber cartridge system prior to lyophilization. The microspheres were bulk lyophilized under sanitary conditions, and stored dry until ready for filling.

### Example 20

### MDI (Metered Dose Inhaler) Container Filling Process

Under sanitary conditions, the appropriate weight of microspheres was added to a stirred pressure vessel and the pressure vessel was charged with the appropriate volume of HFA propellant. The HFA propellant may be P134a, P227, or a blend of the two, or any other propellant(s), alone or in combination, that are useful herein, if required. While the pressure vessel was stirring the HFA, the HFA microsphere suspension was passed through a homogenization loop until a uniform, mono-disperse suspension was achieved. Using a Pamasol or similar aerosol filling line, sterile, pre-crimped, metered dose inhaler cans or vials were charged with the mono-disperse microsphere suspension.

### Example 21

### DPI (Dry Powder Inhaler) Filling Process

Dependent on the particular product, microspheres are supplied as free-flowing microspheres suitable for auger filling or other suitable powder filling technology in the capsules, blister packs, or other suitable containers. Microspheres are added with or without bulking agent. The following bulking agents are used: sodium chloride, lactose, trehalose, sucrose, and/or others that are known to those of skill in the art.

### Example 22

### Determination of Microsphere Particle Size

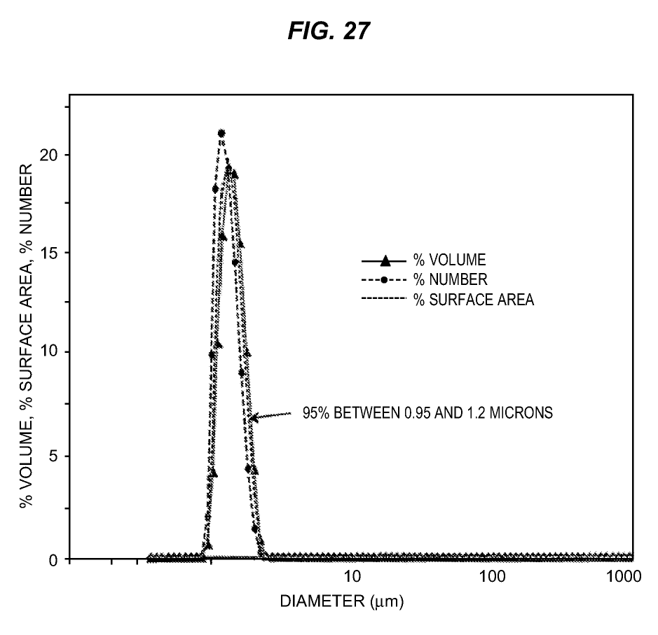

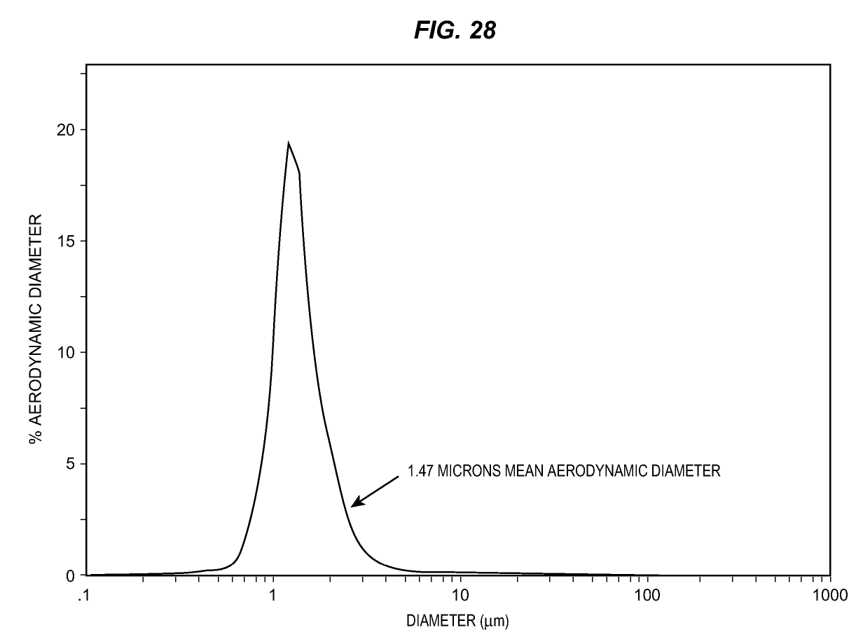

Particle size was determined by light scattering and TSI Aerosizer measurements. The insulin microspheres were mono-dispensed and are approximately 1-1.5 microns in diameter. The results typically show a homogeneous distribution of microspheres with 95% being between 0.95 and 1.20 microns in diameter by number, surface area and volume (FIG. 27). The aerodynamic diameter has been shown to be 1.47 microns in diameter as seen in FIG. 28.

### Example 23

### In Vitro Andersen Cascade Impaction Studies

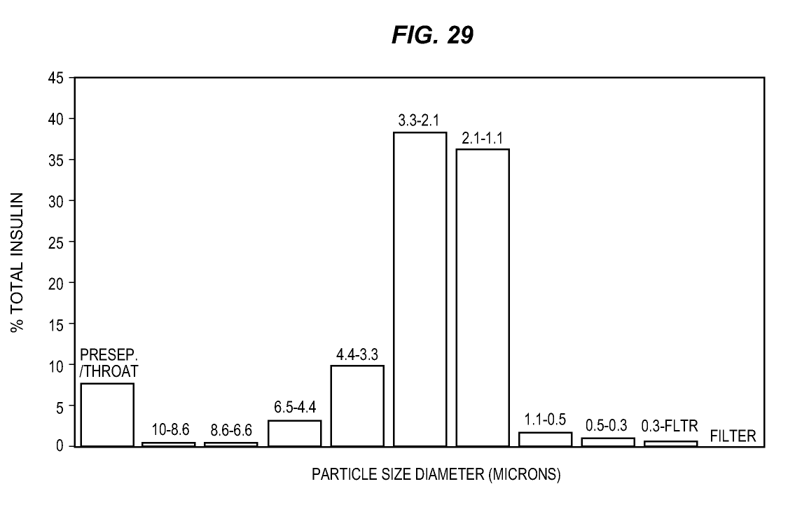

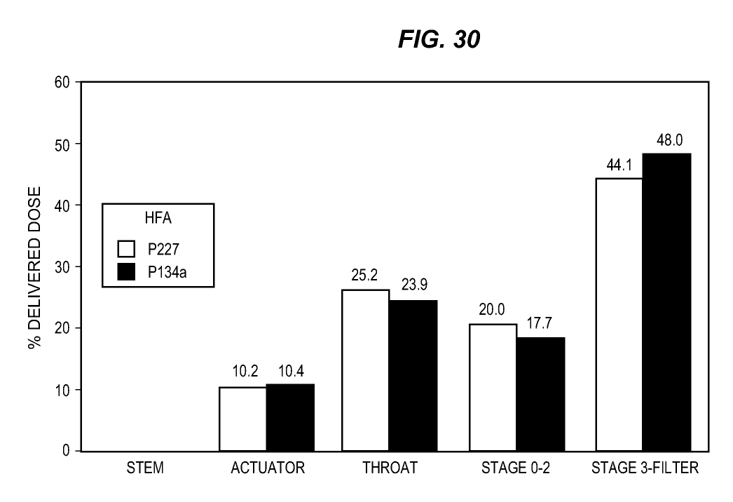

Studies with Insulin microspheres showed that a high “fine particle fraction” (FPF) of microspheres was delivered from Dry Powder Inhalers (>50-60%) (FIG. 29) or from HFA (approximately 40%) (FIG. 30). These represent the fractions of particle sizes that one would expect to penetrate the deep lung. These fine particle fractions are extremely high for even low molecular weight compounds. They have likely not been observed before for any protein drug delivered from a MDI.

“Fine particle fraction” (FPF) is a term of art that refers to the total amount of the drug deposited on the stages in the Andersen cascade impaction studies, within an appropriate particle size range for the drug being tested, divided by the amount total drug delivered from the mouthpiece of the inhaler into the impactor. The FPF for an MDI and a particle which is less than or equal to 4.7 μm; the FPF for a DPI and a particle which is less than or equal to 4.4 μm:

Dry Powder Inhaler: for DPI (60 lpm) a particle size range of ≦4.4 μm Dry Powder Fine Particle Fraction (4.4) is defined as the percentage of the sum of the mass of particles less than or equal to 4.4 microns in diameter divided by the total emitted dose from the device and the mouthpiece of the device.

$$FPF = \frac{\sum Particle Mass \leq 4.4 μ m}{\sum particle mass in all the stages plus mouthpiece} ⨯ 100$$

Metered Dose Inhaler: for MDI (28.3 lpm) a particle size range ≦4.7 μm Metered Dose Inhaler Fine Particle Fraction (4.7) is defined as the percentage of the sum of the mass of particles less than or equal to 4.7 microns in diameter divided by the total emitted dose from the device and the mouthpiece of the device.

$$FPF = \frac{\sum Particle Mass \leq 4.7 μ m}{\sum particle mass in all the stages plus mouthpiece} ⨯ 100$$

According to convention, the FPF is expressed as a percentage.

Geometric Standard Deviation (GSD).

A graph of cumulative percent less than the size range verses the effective cutoff diameter is plotted. From this the diameter at 84.13% and 15.37% are determined. The GSD is calculated as:

GSD=(diameter 84.13%/diameter 15.87%)^{1/2}

Mass Median Aerodynamic Diameter (MMAD).

MMAD=Particle diameter at 50% from the graph above.

Stage Number—F/σ Drug on the stages.

### Example 24

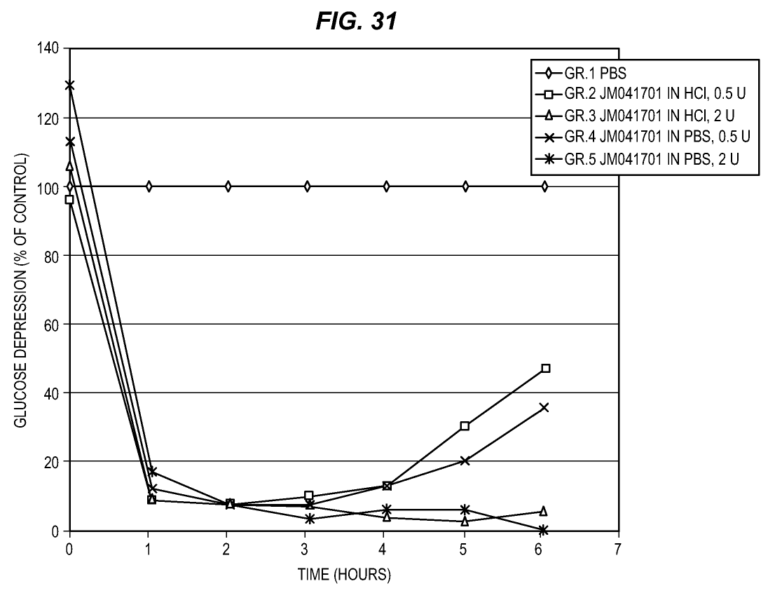

The biological activity of the insulin in Insulin microspheres was demonstrated by injecting the Insulin microspheres suspended in aqueous solutions. FIG. 31 compares the blood glucose depression in normal Fisher rats after insulin injection. The results are expressed as compared to the blood glucose of control rats who received an injection of phosphate buffered saline (PBS) only. FIG. 31 shows that the control animals maintained normal blood glucose concentrations over the 5 hour experiment. Microspheres that were dissolved in HCl (GR.2 and GR.3) depressed blood glucose patterns in a manner similar to intact Insulin microspheres suspended in PBS at the 0.5 Unit (U) dose and at the 2 U dose (GR.4 and GR.5).

### Example 25

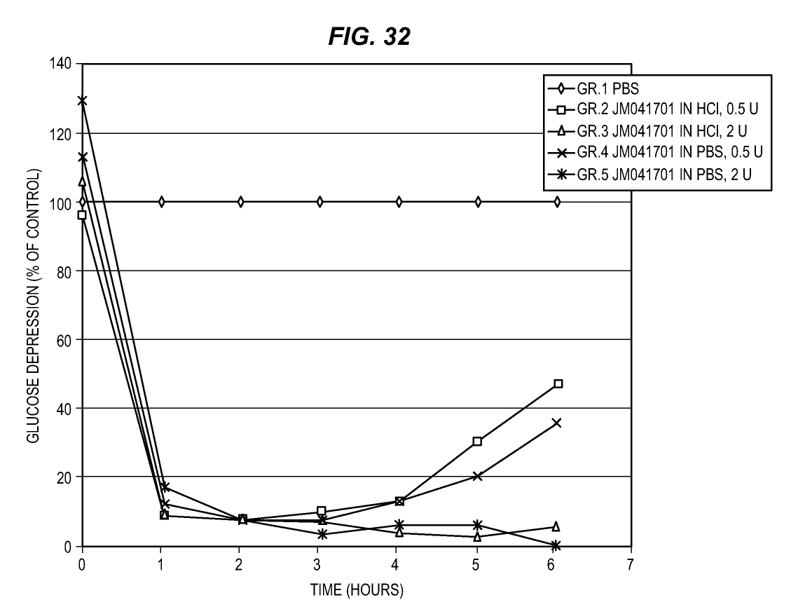

Insulin microspheres were delivered as a solution and as microspheres by directly instilling these formulations intratracheally into the lungs of Fisher rats. FIG. 32 shows that a similar glucose depression pattern was observed for both the insulin solution as well as the insulin microspheres delivered as a suspension (MS YQ051401).

### Example 26

### Metered Dose Inhaler Studies

The Insulin microspheres formulated by the techniques described in the application were then formulated into CFC free propellants for use in Metered Dose Inhalers (MDIs).

Insulin microspheres were added to HFA P134a at several concentrations ranging from 2 mg/ml to 10 mg/dl. The suspension of insulin microspheres was compared to commercially available Proventil albuterrol in HFA P134a.

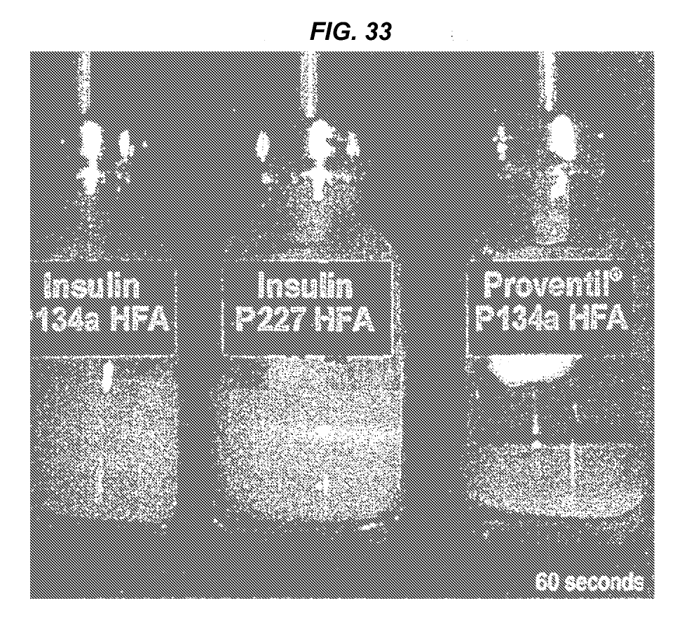

Approximately 20 mg of insulin was weighed into a 20 mL glass vial which was sealed with a valve suitable for dispensing hydrofluoroalkane (HFA) propellants P134a and P227. The addition of the HFAs resulted in the formation of a stable suspension of Insulin microspheres in HFA P134a and HFA P227 as seen in FIG. 33. The Reference Vial contains commercially marketed and FDA-approved Proventil albuterol in HFA P134a. After 60 seconds the Reference Vial completely precipitated to the bottom of the vial. Those skilled in the art will recognize that this represents a source of dose irreproducibility for patients being treated with non-homogeneous suspensions. In contrast, the two vials containing insulin suspended in HFA P134a and HFA P227 remained stable homogeneous suspensions for several minutes. This represents an important property for the dispensing of reproducible dosages of drugs such as insulin from HFA propellants. Stability of insulin microspheres in HFA P134a as assessed by glucose depression in vivo is demonstrated in FIG. 31. Bioactivity of MDA formulation at 4 months was the same as at the time 0.

### Example 27

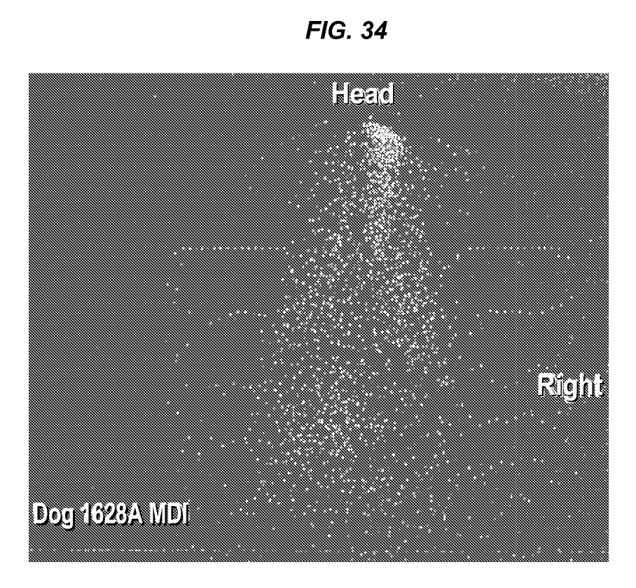

Insulin microspheres were labeled with the Tc-99m radioactive isotope. The Tc-99m insulin was then delivered to the lung of a beagle dog. A gamma camera was used to visualize the distribution of the Tc-99m labeled insulin in the dog lung. FIG. 34 shows the homogeneous distribution of the Insulin microspheres throughout the lung of the lung. This indicates delivery of the microspheres to the lung.

### Example 28

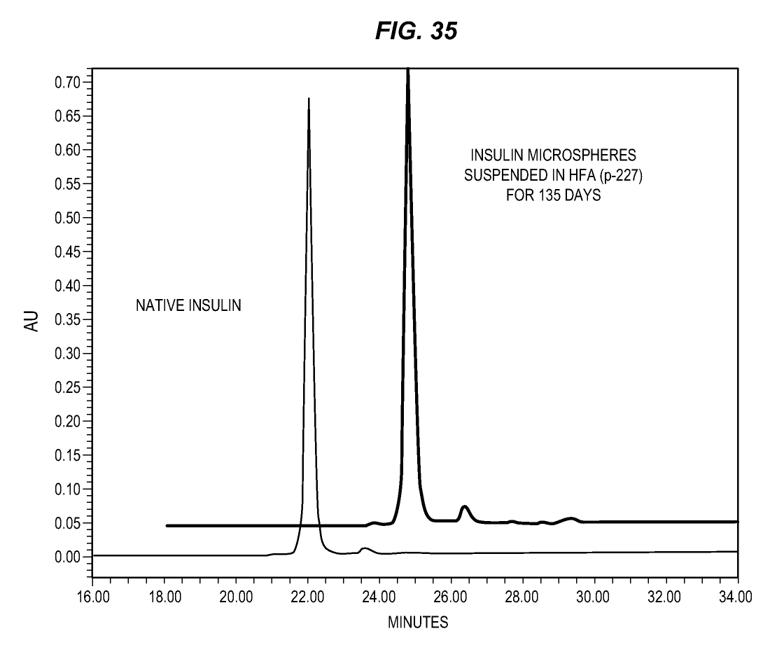

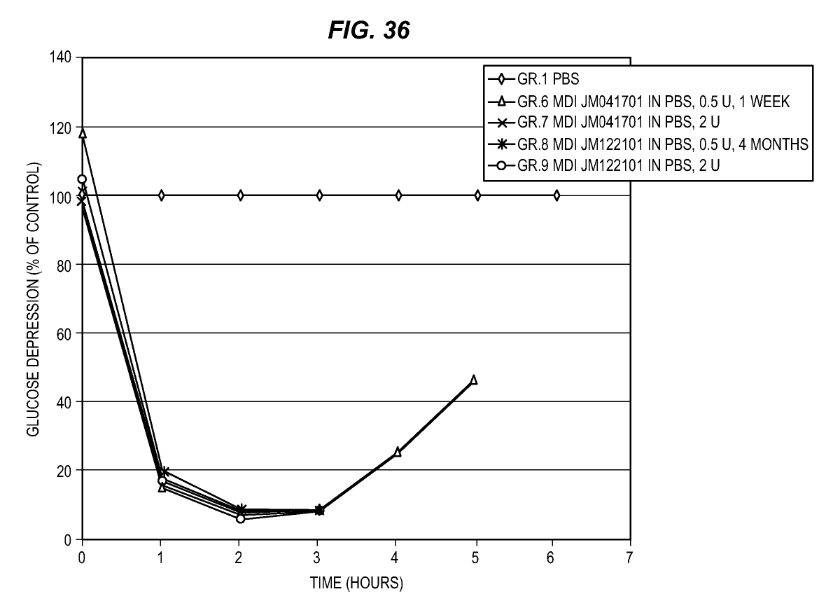

The biochemical integrity and stability of Insulin microspheres suspended in HFA P227 propellant for 135 days was shown by HPLC in FIG. 35. FIG. 36 shows the biological activity of Insulin microspheres stored in HFA for 7 days and 130 days. The insulin was expelled from the MDI device, and resuspended in PBS, and assayed for insulin quantity. Then, 0.5 U and 2 U of each storage time period was instilled intratracheally into Fisher rats. FIG. 36 demonstrates the maintenance of biological activity in vivo.

### Example 29

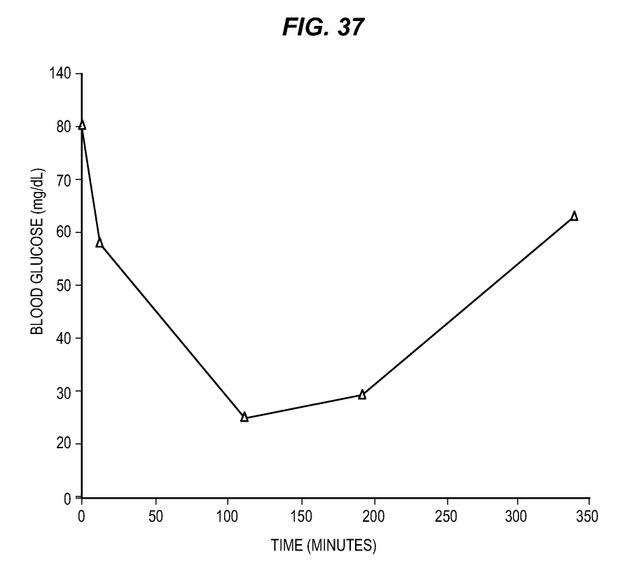

The administration of the biologically active microspheres was demonstrated in dogs. Beagle dogs were anesthetized and placed in an iron lung. Respiration rate was maintained at 75% of the pre-anesthetic breathing rate. Five mg of Insulin microspheres were delivered to the dog using an Aerolizer Dry Powder Inhaler. FIG. 37 shows that a significant reduction in blood glucose was observed within 10 to 15 minutes after the pulmonary administration of the insulin. Hypoglycemic glucose concentration were maintained for over 3 hours before the administration of an oral carbohydrate feeding to the dog.

### Example 30

Six MDIs were submitted for 25° C. stability. After an initial conditioning time of five days at room temperature, upside-down, the samples were assayed by Andersen cascade impactor and DUSA (Dose Unit Sampling Apparatus) at 28 lpm. The DUSA experiments were done in order to determine how much of the expected delivered dose was actually delivered by the device. The data after one month of storage showed that the Andersen results exhibited a majority of the insulin microspheres that were assayed in the 1 to 3 micron sized stages.

The DUSA results showed that the recovered dose at the initial time point was 117±4.7% and at one month the recovered dose was measured to be 106% of the expected dose.

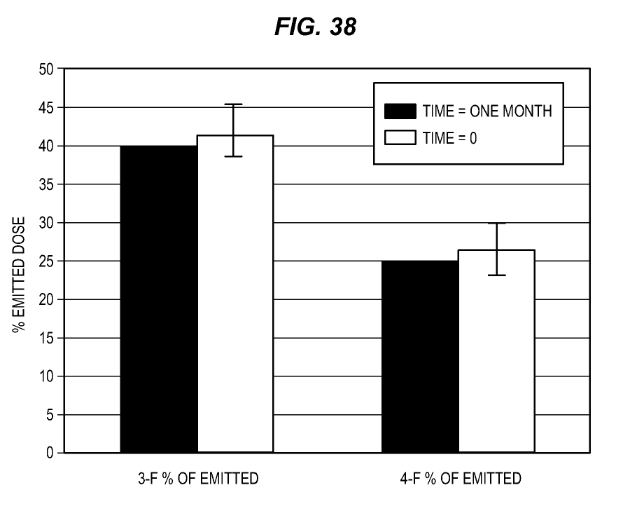

FIG. 38 shows that after one month storage of the Insulin microspheres in HFA P134a, the insulin microspheres deposited in a similar fashion on stages 3 to filter and 4 to filter on the Andersen Cascade Impactor device. This indicates that the aerodynamic properties of the Insulin microspheres appear to remain stable after 1 month storage in HFA. The initial time point is the mean of 6 vials and the one month time point is the value from a single vial.

### Example 31

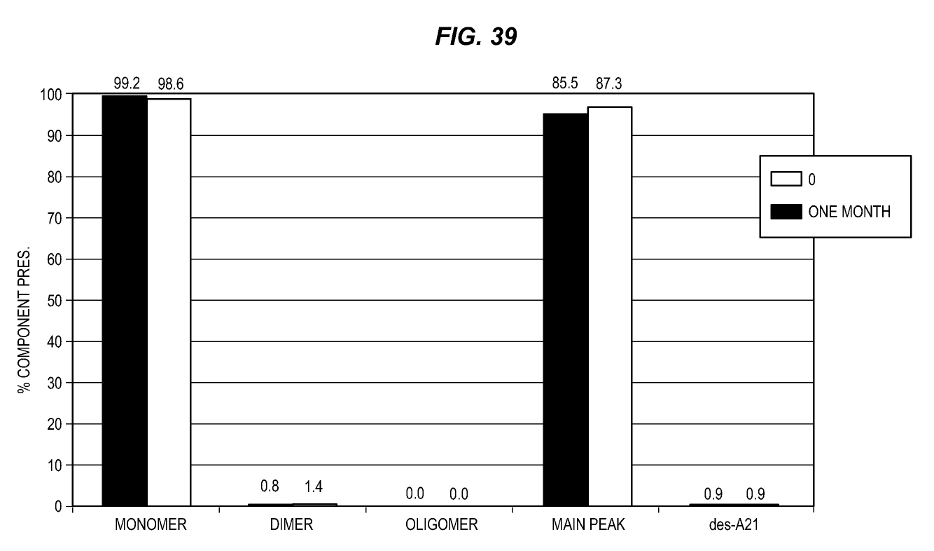

Formulating the microspheres so that they remain biochemically stable in MDI type devices and propellants is a critical component of a MDI based insulin delivery system. The results comparing the biochemical stability of the Insulin microspheres stored in HFA for 1 month showed that the insulin monomer, dimer and oligomer were comparable as well as the main peak and desamido-insulin formation after one month (FIG. 39).

### Example 32

### Preparation of Small Spherical Insulin Particles

A 22.4% (w/w) polyethylene glycol (PEG 3350, NF) solution containing 0.56% (w/w) sodium chloride (USP) and 0.54% (w/w) acetic acid (USP) was prepared with all solutes completed solubilized. The solution was brought to a pH of 5.65±0.05 with 50% sodium hydroxide solution and heated in a stirred-vessel to 75° C. A suspension containing 4 g of Recombinant Human Insulin (USP; Zinc insulin) in USP Purified Water was added to the PEG solution. The contents were mixed for six minutes to dissolve the zinc insulin. This hot solution was delivered to a pre-heated, SS vessel after passing through a 0.2 μm filter. The line was chased with heated USP Purified Water and pumped dry. The resulting solution consists of (all w/w) 16.1% PEG, 0.048% insulin, 0.386% acetic acid, 0.404% NaCl.

The solution was mixed at 50 rpm for additional three minutes to ensure a homogeneous solution. It was then cooled from 70° C. to 20° C. over five minutes at a cooling rate of 10° C./minute, resulting in a spherical insulin particle suspension. This cooling was achieved by feeding a coolant (i.e., 2° C. water) through an internal coil and the vessel jacket.

Two liters of a zinc chloride buffer (0.026% zinc chloride/0.16% acetate pH 7.0±0.05) were added to the resulting microsphere suspension. A constant volume wash was executed by recirculating suspension from the vessel through a 750 KD Ultrafiltration Hollow Fiber Cartridge via a peristaltic pump at shear rates of approximately 4000 sec⁻¹. Seven volume exchanges were carried out to remove polyethylene glycol (PEG) from the suspension. The PEG-reduced suspension was then concentrated to 5 liters, and another constant volume (5×) washing step with USP Purified Water was carried out to remove any remaining PEG and residual salts.

The PEG-free suspension was concentrated a second time to 1.5 liters where it was then drained into a collection bottle. This microsphere suspension was poured into filter-topped, SS lyophilization trays and freeze-dried. Upon completion of the freeze-drying cycle the lyophilized spherical insulin particles were collected and stored in bulk prior to filling of the inhaler.

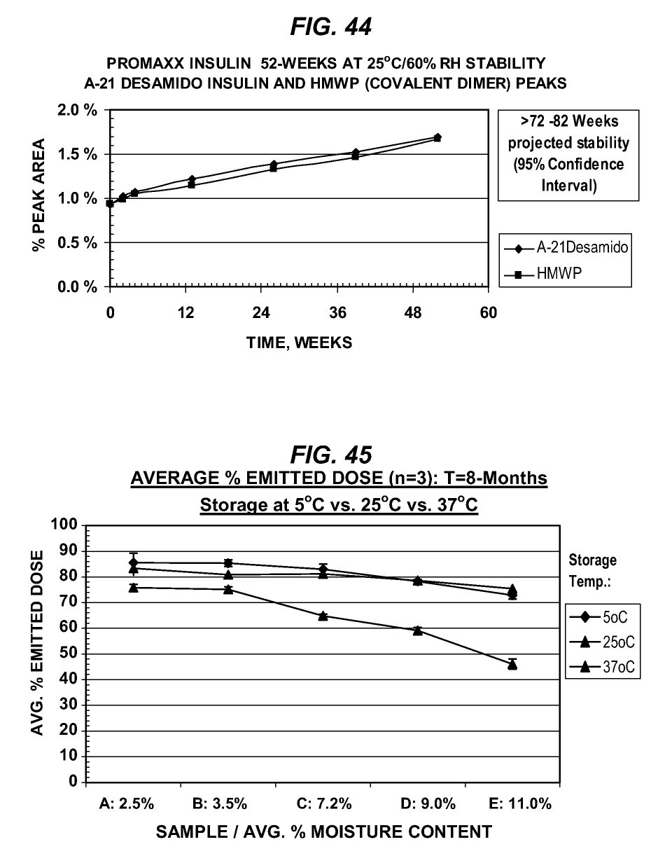

The dry insulin microspheres had a mean insulin content (w/w) of 94.7% (ranging from 93.3% to 95.8%), a mean moisture content (w/w) of 3.6% (ranging from 2.4% to 4.9%), and a mean zinc content (w/w) of 1.4% (ranging from 1.1% to 1.8%). Among the microspheres, 50% of the population had a particle size of less than or equal to 1.7±0.1 microns, and 95% of the populations had a particle size of less than or equal to 2.6±0.3 microns. Using the criteria that insulin microspheres having a % A-21 desamido peak area of 2% or less and/or a % high molecular weight product peak area of 2% or less are deemed stable, the insulin microspheres kept in sealed vials stored at 25° C. and 60% relative humidity were projected to be stable for at least 72 wks to 82 wks at the 95% confidence interval (FIG. 44). The insulin microspheres could be stored at or below 37° C. (e.g., 25° C., 5° C.) over long periods of time (e.g., 8 months) without significant deterioration in their aerodynamic properties (e.g., emitted dose at 70% or greater at the end of 8-months storage), as long as their moisture content is kept at or under 5% (FIG. 45). The insulin microspheres with moisture content greater than 5% would be stored at lower temperatures (e.g., 25° C., 5° C.) to retain the aerodynamic properties (e.g., emitted dose at 70% or greater at the end of 8-months storage), in the absence of further increase in moisture content (FIG. 45).

### Example 33

### Pulmonary Delivery of Recombinant Human Insulin Inhalation Powder (RHIIP) in Human Subjects

RHIIP was prepared according to the method set forth in Example 32.

The human subjects were healthy male volunteers aged 18-40, with normal pulmonary function, body mass index between 18 and 27 kg/m², a weight of between 60 to 90 kg and the ability to achieve target inspiratory flow rate of 90±30 L/min through a Cyclohaler™ dry powder inhaler (Pharmachemie, Haarlem, the Netherlands). Subjects were excluded from the study if they presented any of the following symptoms: active or chronic pulmonary disease; a history of diabetes, impaired glucose tolerance or impaired fasting glucose; a fasting plasma glucose of >100 mg/dL; a fasting HbA1c of >6.0%; known or suspected allergy to insulin or any components of the formulation; a positive anti-insulin antibody of >10 U/ml.

Meeting the above criteria, thirty healthy male subjects (30±1.1 years (mean ±SEM), BMI 24.2±0.3 kg/m²) were included in a single-center, open label, randomized, active controlled, two-way crossover study, and received 10 IU human insulin via subcutaneous injection as control (SC, Actrapid® by Novo Nordisk, Denmark), and 6.5 mg (single dose) of RHIIP (187 IU) contained in HPMC capsules (size 3 Vcaps™ by Capsugel, Bornem, Belgium; see FIGS. 43b-c) and delivered via a commercially available DPI designed to deliver medications to the upper airways of the lung (Cyclohaler™ by Pharmachemie, Haarlem, the Netherlands; see FIGS. 43a-c) under euglycemic glucose clamp conditions (clamp level 5 mmol/L, continuous iv insulin infusion of 0.15 mU/kg/min, clamp duration 10 h post-dosing). Subjects were trained to inhale RHIIP with an inhalation flow rate of 90±30 L/min prior to the clamp experiments.

The following table shows the exemplary product release data of the RHIIP compositions used in the study:

Batch Release Data

| Mean Capsule Fill Weight | 7.1 mg RHIIP |
|---|---|
| Mean Moisture Content | 8.6% |
| Mean Potency | 27.8 IU per mg |
| | 96% (anhydrous) |
| Mean Emitted Dose | 76.5% |
| Fine Particle Fraction | 75.8% |

Each of the dosing visits had a minimum duration of 12 hours of confinement to the clinic during which the subject was monitored for any adverse effects. There was an interval of 72 hours to 14 days between the two dose administrations. After the study, a medical examination was performed within 3 to 14 days after the second dosing visit.

Determination of Pharmacokinetic parameters: The following pharmacokinetic parameters were calculated from the serum insulin profiles with and without adjustment for the intravenous basal insulin infusion: AUC_{1-10 hour}; AUC_{0-1.5 hours}; AUC_{0-3 hours}, Cₘₐₓ, Tₘₐₓ, T_{10% AUC(1-10 hours)}, T_{90% AUC(0-10 hours)}, duration of insulin appearance and relative bioavailability.

Determination of Pharmacodynamic parameters: The following pharmacodynamic parameters were calculated from baseline adjusted GIR profiles AUC_{GIR(0-10 hour)}, AUC_{GIR(0-1.5 hours)}; AUC_{GIR(0-3 hours)}; GIRₘₐₓ; T_{maxGR}; early and late T₅₀, T_{10% incGIRmax}, T_{10% decGIRmax}, T₁₀% AUC_{GIR(0-10 hour)}, T_{90% AUCGIR(0-10 hour)}, duration of insulin action, the total amount of glucose administered from 0 to 10 hours and the relative biopotency.

For Safety screening, the subjects were routinely questioned at each study visit about the occurrence of any adverse events and the use of any concomitant medicaments. Pulmonary function tests (PRT) and blood samples for haematology were obtained at the screening visit and post-study medical examination visits as well as before and after (PFT only) each treatment. Vital signs were measured at specified times points pre- and post-dose. Electrocardiograms, serum biochemistry and urinalysis were performed at the screening visit and post-study medical examination.

During each study period an euglycemic glucose clamp method was used to ensure that the blood glucose was maintained at a pre-determined level and also acted as a surrogate marker for the pharmacodynamic effect of the exogenously added insulin. The blood samples were taken at specified time points during the course of the study period in order to measure insulin and C-peptide concentrations. The inhalation profiles were obtained during RHIIP administration and safety tests were monitored. For the safety monitoring, vital signs (respiration, pulse, temperature and blood pressure), electrocardiograms, pulmonary function tests, haematology and clinical chemistry were measured at specified time-points during the course of the study. Adverse events and concomitant medications were recorded throughout the study.

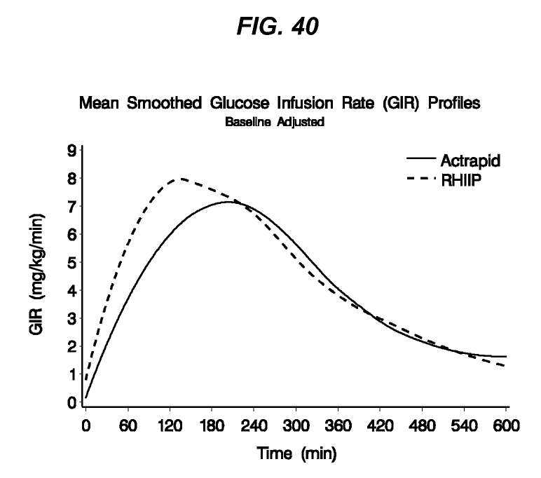

Inhalation of RHIIP was surprisingly well tolerated. In particular not a single episode of cough or shortness of breath occurred during dosing with RHIIP. RHIIP showed a faster onset of action than subcutaneous administration (time to reach 10% of the total area under the glucose infusion rates (GIR) curves being 73±2 min vs. 95±3 min; GIR-tₘₐₓ being 173±13 min vs. 218±9 min, p<0.0001). Duration of action (371±11 min vs. 366±7 min) and total metabolic effect (GIR-AUC_{0-10 h} being 2734±274 mg/kg vs. 2482±155 mg/kg) were comparable (FIG. 40). Pharmacokinetic results were in accordance with these pharmacodynamic findings: RHIIP was absorbed faster (time to reach 10% of the total area under the serum insulin level (INS) curves being 44±3 min vs. 66±3 min, p<0.0001), and maximum serum insulin levels were reached earlier (86±10 min vs. 141±12 min, p=0.002), in comparison to SC. Relative bioavailability of RHIIP was 12±2%; relative biopotency was 6±1%.

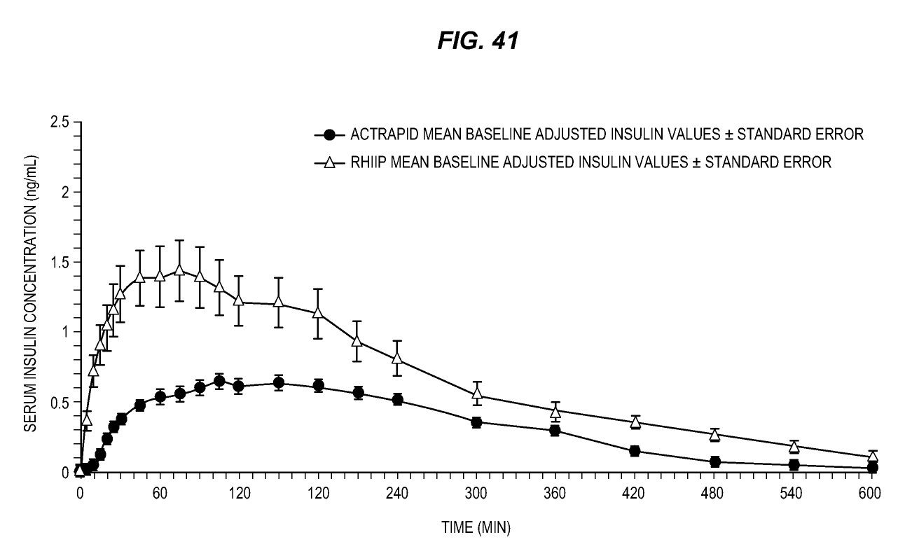

The following table presents a summary of the pharmacokinetic parameters determined in this study. The data show baseline adjusted pharmacokinetic parameters (mean ±SE) by treatment for all randomized subjects. The pharmacokinetic data is graphically depicted in FIG. 41, which shows that the serum insulin concentration achieved with RHIIP was higher than that achieved with Actrapid® SC administration and remained higher throughout the monitored period of 10 hours. These data show that the RHIIP oral inhalation dose had an earlier onset and similar duration of absorption as compared to the Actrapid® subcutaneous dose.

| | | | | Overall |
|---|---|---|---|---|
| | | | | pValue for |
| Parameter | Units | RHIIP | SC Insulin | treatment |
| AUC_{0-1.5 hr} | (ng/mL)min | 107.8 ± 16.13 | 35.5 ± 3.18 | 0.0008 |
| AUC_{0-3 hr} | (ng/mL)min | 219.1 ± 31.56 | 91.8 ± 7.51 | 0.0008 |
| AUC_{0-10 hr} | (ng/mL)min | 410.5 ± 56.57 | 198.0 ± 11.22 | 0.0013 |
| Tₘₐₓ | min | 86.2 ± 9.89 | 141.0 ± 11.63 | 0.0017 |
| T_{10% AUC (0-10 hr)} | min | 44.3 ± 2.74 | 66.0 ± 2.75 | <0.0001 |
| T_{90% AUC (0-10 hr)} | min | 393.2 ± 15.65 | 383.3 ± 11.46 | 0.5787 |
| Duration of | min | 348.8 ± 14.04 | 317.3 ± 10.39 | 0.0531 |
| insulin appearance | | | | |
| Relative | % | 12.0 ± 1.80 | NA | NA |
| Bioavailability | | | | |

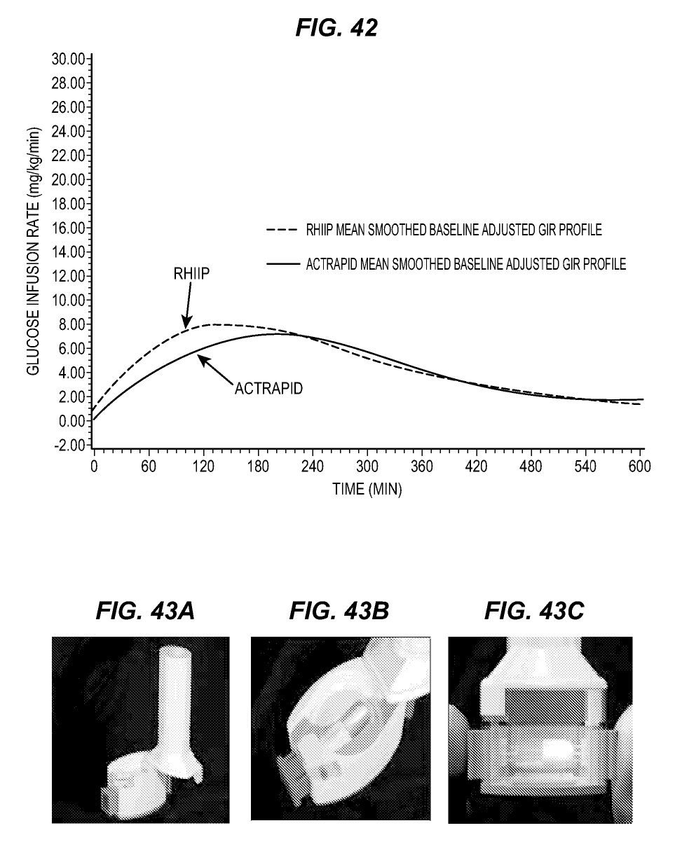

The following table presents a summary of the pharmacodynamic parameters determined in this study. The data show baseline adjusted pharmacodynamic parameters (mean ±SE) by treatment for all randomized subjects. The pharmacodynamic data is further depicted in FIG. 42.

| | | | | Overall |
|---|---|---|---|---|
| | | | | pValue for |
| Parameter | Units | RHIIP | SC Insulin | treatment |
| AUC_{GIR (0-1.5 hr)} | mg/kg | 417.5 ± 49.32 | 252.5 ± 26.83 | 0.0007 |
| AUC_{GIR (0-3 hr)} | mg/kg | 1107.2 ± 121.66 | 813.8 ± 69.97 | 0.0075 |
| AUC_{GIR (0-10 hr)} | mg/kg | 2733.8 ± 274.35 | 2482.2 ± 154.78 | 0.3257 |
| T_{maxGIR} | min | 173.0 ± 13.13 | 218.0 ± 8.94 | <0.0001 |
| T_{10% AUCGIR(0-10 hr)} | min | 73.3 ± 2.44 | 94.9 ± 3.20 | <0.0001 |
| T_{90% AUCGIR(0-10 hr)} | min | 444.2 ± 11.99 | 460.8 ± 8.53 | 0.0160 |
| Duration of | min | 370.8 ± 11.10 | 365.9 ± 7.01 | 0.1883 |
| insulin appearance | | | | |
| Relative | % | 6.3 ± 0.63 | NA | NA |
| Bioavailability | | | | |

Throughout the study adverse events were monitored for the subjects being treated and the following table summarizes the treatment-emergent adverse events:

| Adverse Event | RHIIP N = 30 | Actrapid N = 30 |
|---|---|---|
| Cough | 0 | 0 |
| Shortness of Breath | 0 | 0 |
| Emesis | 0 | 1 |
| Dizziness | 1 | 0 |
| Headache | 1 | 0 |
| Cold | 0 | 1 |
| Redness of Pharynx | 1 | 0 |
| Phlebitis | 0 | 2 |

The delivery of the spherical insulin particles disclosed herein to the deep lung with an off-the-shelf DPI designed for drug delivery to the upper airways was safe and efficacious. RHIIP showed a fast onset of action and a bioavailability comparable to that reported for other inhaled insulin formulations using specifically designed devices. For the inhaled insulin administration, there was no reported incidence of either coughing or shortness of breath either immediately upon inhalation, or throughout the 10-hour post-dosing monitoring period. The most common adverse occurrence was phlebitis and it was seen in subjects receiving Actrapid® and not the RHIIP.

The baseline adjusted relative bioavailability for a single 6.5 mg dose oral inhalation of RHIIP (prepared according to Example 32) was estimated to be 12% with respect to a single 10 IU dosing of subcutaneously administered insulin. In addition, while the duration of insulin action was similar between subcutaneous administration of Actrapid® and pulmonary administration of RHIIP, the onset of insulin action was earlier with RHIIP than with Actrapid®.

### Example 34

### Single Dose Inhalation of Insulin Microparticles in Beagle Dogs

Pharmacokinetics and relative bioavailability of insulin microspheres were evaluated after a single dose faced-maneuver inhalation or subcutaneous administration in 6 male Beagle dogs. Dry powder insulin microspheres were administered via inhalation at dose levels 0.6 mg per animal and 1.6 mg per animal, while Humulin® insulin was administered via subcutaneous injection of 0.15 mg per animal (0.35 U/kg). The serum insulin concentration was corrected C-peptide using Equation 1 reported by Home et al. (Eur. J. Clin. Pharmacol. 55 (1999): 199-203). Any negative numbers in the C-peptide corrected insulin data were treated as 0 for the pharmacokinetic analysis, which used a noncompartmental model with WinNonlinPro® version 4.1 (Pharsight Corp., Mountain View, Calif.).

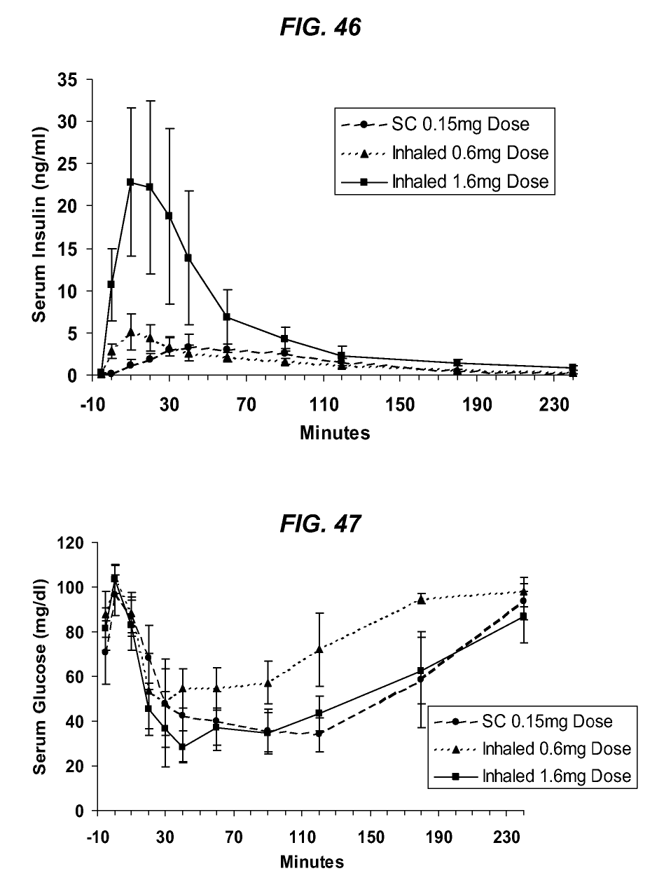

Inhalation administration at both doses increased serum insulin concentration for 10-20 minutes (FIG. 46). The serum concentration declined thereafter, resulting in an apparent bi-exponential concentration-time curve. The apparent volume of distribution and the mean residence time of serum insulin in both dose groups were similar. FIG. 47 shows that a significant reduction in blood glucose was observed within 10 to 15 minutes post dosing. Hypoglycemic glucose concentrations were maintained for 4 hours.

With a 3-fold increase in dosing, the 1.6 mg dose resulted in a mean peak serum insulin concentration (Cₘₐₓ) of 24±10 ng/ml at 13±5 minutes (tₘₐₓ) post dosing, which was 5-folder greater than that of the 0.6 mg dose (5±1.8 ng/ml at 12±4 minutes post dosing), and a mean area under the serum insulin concentration−time curve from 0 to infinity (AUC_{0-∞}) of 1504±544 ng·min/ml, which was 4-fold greater than that of the 0.6 mg dose (411±93 ng·min/ml). This indicated that the extent of increase in systemic exposure to insulin following inhalation of the insulin microspheres was greater than the extent of increase in administered dose. At the 1.6 mg dose, the mean residence time (MRT) was 77±22 minutes and the apparent elimination half-life (t_{1/2}) was 67±22 minutes, comparable to those of the 0.6 mg dose (94±22 minutes and 67±13 minutes, respectively). The relative bioavailability of the 1.6 mg dose compared to the subcutaneous insulin was close to 40% (39%), while the relative bioavailability of the 0.6 mg dose was close to 30% (29%). In comparison, the subcutaneous injection resulted in a Cₘₐₓ of 3.8±1.0 ng/ml, a tₘₐₓ of 58±26 minutes, and a t_{1/2}, of 32±6 minutes. This indicated that the insulin microsphere inhalation formulations were faster (tₘₐₓ being shorter) in delivering insulin to the body and allowed the insulin to stay longer (t_{1/2} being longer) in the body, as compared to subcutaneous administration.

What is claimed is:

1. A composition for the pulmonary delivery of insulin through a powder dispenser comprising a powder that comprises a dose of insulin, said powder consisting essentially of solid, substantially spherical insulin particles, the insulin particles comprising at least 90% by weight insulin suitable for in-vivo delivery and having a density of from about 0.50 to about 2.00 g/cm³.

2. The composition of claim 1, wherein the solid, small spherical microparticles of insulin have a density of from about 0.50 to about 1.5 g/cm³.

3. The composition of claim 1, wherein the solid, small spherical microparticles of insulin have a density greater than 0.75 g/cm³.

4. The composition of claim 1, wherein the solid, small spherical microparticles of insulin have a density greater than 0.85 g/cm³.

5. The composition of claim 1, wherein the solid, small spherical microparticles further comprise an excipient to enhance the stability of the solid, small spherical particles, to provide controlled release of the solid, small spherical particle, or to enhance permeation of the solid, small spherical particles through biological tissues, said excipients being present in said microparticles at less than 5% by weight.

6. The composition of claim 1, wherein the excipient is selected from the group consisting of: carbohydrates, cations, anions, amino acids, lipids, fatty acids, surfactants, triglycerides, bile acids or their salts, fatty acid esters, and polymers.

7. The composition of claim 6, wherein the cation is selected from group consisting of Zn²⁺, Mg²⁺, and Ca²⁺.

8. The composition of claim 1, wherein 90% of the small spherical microparticles have size between from about 0.01 μm to about 5 μm.

9. The composition of claim 1, wherein 90% of the small spherical microparticles have a size between from about from about 0.1 μm to about 5 μm.

10. The composition of claim 1 wherein 90% the small spherical microparticles have a size between from about 1 μm to about 3 μm.

11. The composition of claim 1, wherein the narrow size distribution comprises the ratio of a volume diameter of the 90^{th} percentile of the small spherical particles to the volume diameter of the 10^{th} percentile is less than or equal to about 5.0.

12. The composition of claim 1, wherein the insulin is from about 95% to about 100% by weight of the microparticles.

13. The composition of claim 1, wherein the small spherical particles are semi-crystalline or non-crystalline.

14. The composition of claim 1, wherein the microparticles are microspheres comprising greater than about 99% insulin by weight.

15. The composition of claim 14, wherein the composition does not comprise a surfactant.

16. The composition of claim 14, wherein the composition does not comprise an excipient and contains only the insulin microspheres.

17. A composition for the pulmonary delivery of insulin through a powder dispenser comprising a carrying member to be used in connection with the powder dispenser, said carrying member carries a powder consisting essentially of solid, substantially spherical insulin particles, the insulin particles comprising at least 90% by weight insulin suitable for in-vivo delivery and having a density of from about 0.50 to about 2.00 g/cm³.

18. The composition of claim 17, wherein the solid, small spherical microparticles of insulin have a density of from about 0.50 to about 1.5 g/cm³.

19. The composition of claim 17, wherein the solid, small spherical microparticles of insulin have a density greater than 0.75 g/cm³.

20. The composition of claim 17, wherein the solid, small spherical microparticles of insulin have a density greater than 0.85 g/cm³.

21. The composition of claim 17, wherein the solid, small spherical microparticles further comprise an excipient to enhance the stability of the solid, small spherical particles, to provide controlled release of the solid, small spherical particle, or to enhance permeation of the solid, small spherical particles through biological tissues, said excipients being present in said microparticles at less than 5% by weight.

22. The composition of claim 17, wherein the excipient is selected from the group consisting of: carbohydrates, cations, anions, amino acids, lipids, fatty acids, surfactants, triglycerides, bile acids or their salts, fatty acid esters, and polymers.

23. The composition of claim 22, wherein the cation is selected from group consisting of Zn²⁺, Mg²⁺, and Ca²⁺.

24. The composition of claim 17, wherein 90% of the small spherical microparticles have size between from about 0.01 μm to about 5 μm.

25. The composition of claim 17, wherein 90% of the small spherical microparticles have a size between from about from about 0.1 μm to about 5 μm.

26. The composition of claim 17, wherein 90% the small spherical microparticles have a size between from about 1 μm to about 3 μm.

27. The composition of claim 17, wherein the narrow size distribution comprises the ratio of a volume diameter of the 90^{th} percentile of the small spherical particles to the volume diameter of the 10^{th} percentile is less than or equal to about 5.0.

28. The composition of claim 17, wherein the insulin is from about 95% to about 100% by weight of the microparticles.

29. The composition of claim 17, wherein the small spherical particles are semi-crystalline or non-crystalline.

30. The composition of claim 17, wherein the microparticles are microspheres comprising greater than about 99% insulin by weight.

31. The composition of claim 30, wherein the composition does not comprise a surfactant.

32. The composition of claim 30, wherein the composition does not comprise an excipient and contains only the insulin microspheres.

33. A method of administering insulin to the pulmonary system of a subject, comprising: administering to the pulmonary system an amount of the composition of claim 1 effective to produce a change in the subject's serum insulin level or the subject's serum glucose level or both, wherein the administration of said composition does not produce coughing in said subject upon inhalation.

34. A composition for pulmonary delivery of insulin, comprising a powder that comprises a dose of insulin, said powder consisting essentially of solid, substantially spherical insulin particles, the insulin particles comprising at least 90% by weight insulin suitable for in-vivo delivery and having a density of from about 0.50 to about 2.00 g/cm³, wherein said composition do not produce coughing in healthy male subjects upon pulmonary administration at an insulin dose of 6.5 mg.

35. A method of administering insulin to the pulmonary system of a subject, comprising: administering to the respiratory tract of a subject in need of treatment, an effective amount of the composition of claim 1, wherein the administration of said composition does not produce shortness of breath in said subject upon inhalation.

36. The method of claim 33, wherein the administration produces a bioavailability of said insulin bioavailability of at least 10% of the bioavailablilty produced by a subcutaneous dose.

37. The method of claim 34, wherein the administration produces a bioavailability of said insulin bioavailability of at least 10% of the bioavailablilty produced by a subcutaneous dose.

38. The method of claim 35, wherein the administration produces a bioavailability of said insulin bioavailability of at least 10% of the bioavailablilty produced by a subcutaneous dose.

39. The method of claim 33, wherein the administration produces a bioavailability of said insulin bioavailability of at least 12% of the bioavailablilty produced by a subcutaneous dose.

40. The method of claim 34, wherein the administration produces a bioavailability of said insulin bioavailability of at least 12% of the bioavailablilty produced by a subcutaneous dose.

41. The method of claim 35, wherein the administration produces a bioavailability of said insulin bioavailability of at least 12% of the bioavailablilty produced by a subcutaneous dose.

42. The method of claim 33, wherein the administration produces a bioavailability of said insulin bioavailability of at least 15% of the bioavailablilty produced by a subcutaneous dose.

43. The method of claim 34, wherein the administration produces a bioavailability of said insulin bioavailability of at least 15% of the bioavailablilty produced by a subcutaneous dose.

44. The method of claim 35, wherein the administration produces a bioavailability of said insulin bioavailability of at least 15% of the bioavailablilty produced by a subcutaneous dose.

45. A method of achieving a deep lung deposition of insulin in a subject comprising administering to the pulmonary system of said subject a composition of claim 1.
